(19) United States
(12) Patent Application Publication
(10) Pub. No.: US 2022/0323641 A1
(43) Pub. Date: Oct. 13, 2022

(54) MATERIALS FOR DELIVERY OF TETHERABLE PROTEINS IN BONE IMPLANTS

(71) Applicant: Theradaptive, Inc., Frederick, MD (US)

(72) Inventors: Luis ALVAREZ, Lexington, MA (US); Todd HEIL, Lexington, MA (US)

(21) Appl. No.: 17/636,527

(22) PCT Filed: Aug. 19, 2020
(86) PCT No.: PCT/US2020/047042

§ 371 Date: Feb. 18, 2022

Related U.S. Application Data
(60) Provisional application No. 62/889,296, filed on Aug. 20, 2019.

(51) Int. Cl. A61L 27/10; A61L 27/42; A61L 27/54; A61L 27/56; A61K 38/08; A61K 38/10; A61K 38/17; A61K 38/18

(52) U.S. Cl. CPC A61L 27/10; A61L 27/425; A61L 27/54; A61L 27/56; A61K 38/08; A61K 38/10; A61K 38/1709; A61K 38/1808; A61K 38/1825; A61K 38/1858; A61L 2300/414; A61L 2430/02; A61L 2430/38

(57) ABSTRACT

The present disclosure provides devices comprising a therapeutic agent bound to a printed three-dimensional structure. The printed three-dimensional structure comprises about 50% to about 100% by weight ceramic and about 0% to about 50% by weight N polymer. Ink formulations for three-dimensional printing are also disclosed. Additionally, provided herein are methods for manufacturing devices and uses thereof, e.g., in treating a condition in a subject in need thereof.

### CROSS-REFERENCE TO RELATED APPLICATIONS

This application claims the benefit of U.S. Provisional Patent Application Ser. No. 62/889,296 filed Aug. 20, 2019, which is incorporated herein by reference in its entirety.

### GOVERNMENT RIGHTS

This invention was made with government support under W81XWH-18-C-0182 awarded by the US Army Medical Material Command. The government has certain rights in the invention.

### SEQUENCE LISTING

The application contains a Sequence Listing which has been submitted in ASCII format via EFS-Web and is hereby incorporated by reference in its entirety. The ASCII copy, created on Aug. 13, 2020, is named 50222-703.601_SEQ.txt and is 235 KB in size.

### BACKGROUND

Three-dimensional (3D) printing is a manufacturing process of making three dimensional solid objects from a digital file. In the additive process of 3D printing an object is created by laying down successive layers of material until the desired object is created, with up to micrometer accuracy. Paired with computer-aided design (CAD) software, 3D printing enables production of complex functional shapes that can be easily customized compared with traditional manufacturing methods.

Surgically implanting scaffolds and/or other forms of graft materials to promote tissue regeneration is a useful technique if the implant can match the mechanical properties of the native tissue. Various materials can be used as ink in the fabrication of porous 3D-printed structures for implantation, including materials that mimic tissue and enable tissue regrowth. Inks with effective bioactive and mechanical properties are required for regeneration of native tissue, and if used in 3D printing can be customized and adopted for large tissue defect repair.

### SUMMARY

Various ink formulations incorporating a bioactive ceramic material for 3D printing of porous bone scaffold implants have been developed. These inks are formulated by dispersing ceramic particles into the liquid ink components with a dual asymmetric centrifugal mixing process. The scaffolds printed with these inks are subsequently coated with tethered proteins that promote bone growth. The 3D-printed materials can also be seeded with cells and used for surgical bone replacement and grafting. One of the developed inks enables 3D-printing of a rigid calcium phosphate ceramic material, another a calcium phosphate-polymer composite material that is flexible and easily handled, and a third that contains a blend of tricalcium phosphate powder, a non-water-soluble polymer, and a water-soluble polymer that produces a printed material that is porous and flexible. The ink formulations possess a shear-thinning behavior that enables 3D printing via extrusion-based techniques such as Direct Ink Writing (also known as robocasting) and melt extrusion.

Both techniques are methods of additive manufacturing in which a filament of a paste (called the “ink”) is extruded from a small nozzle (e.g., from a syringe) while the nozzle is moved across a build platform. A 3D computer aided design (CAD) model of the object to be printed is divided up into layers of similar thickness as the nozzle diameter. The object is produced by extruding a filament of ink in the shape required to fill the first layer. Then either the build platform is moved down or the nozzle is moved up (e.g., by the width of the nozzle diameter) and the next layer is deposited in the pattern required by the subsequent layer. This process is repeated until the fabrication of the 3D object is complete.

In robocasting, as the nozzle's position is controlled to draw out the shape of each layer, the ink (typically a ceramic slurry) exits the nozzle in a liquid-like state but retains its shape immediately due to the rheological property of shear thinning of the ink. The desired object is thus built layer by layer, and geometrically complex ceramic green bodies can be produced.

During melt extrusion, the ink material is heated above the polymer melting temperature while in the 3D printer so as to become extrudable through the nozzle. After the polymer exits the print nozzle, it quickly cools and hardens which enables subsequent layers to be applied to fabricate a three-dimensional object.

An aspect of the present disclosure is a device comprising a therapeutic agent non-covalently bound to a printed three-dimensional structure. The printed three-dimensional structure comprises about 50% to about 100% by weight ceramic and about 0% to about 50% by weight polymer.

In some embodiments, the three-dimensional structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

In various embodiments, the ceramic comprises calcium phosphate, hydroxyapatite, fluorapatite, bone, silicate, or vanadate, or a combination thereof.

In embodiments, the ceramic comprises beta-tricalcium phosphate (β-TCP).

In some embodiments, the polymer comprises polycaprolactone.

In various embodiments, the device comprises about 100% by weight ceramic. In the device, the ceramic may comprise beta-tricalcium phosphate (β-TCP).

In embodiments, the devices comprise about 70% to about 80% by weight ceramic, and about 20% to about 30% by weight polymer. In the device, the ceramic may comprise beta-tricalcium phosphate (β-TCP) and the polymer comprises polycaprolactone.

The printed three-dimensional structure may be formed from an ink comprising about 30% to about 70% by weight the ceramic, about 5% to about 30% by the weight polymer, and optionally an anti-foaming agent and/or a dispersing agent.

In some embodiments, the therapeutic agent comprises a mammalian growth factor or a functional portion thereof.

In various embodiments, the therapeutic agent comprises one or more polypeptides selected from Table 4, or a functional portion thereof.

In embodiments, the therapeutic agent comprises a bone morphogenetic protein (BMP).

The therapeutic agent may comprise a targeting moiety, and the targeting moiety is non-covalently bound to the printed three-dimensional structure. In the device, the targeting moiety may be bound to the printed three-dimensional structure with an affinity of about 1 pM to about 100 μm. The targeting moiety may comprise a polypeptide at least about 70%, 75%, 80%, 85%, 90%, 95%, or 100% identical to any one of the sequences of Tables 5-6. In the device, the targeting moiety may comprise about 2, 3, 4, 5, 6, 7, 8, 9, or 10 sequences selected from the sequence of Tables 5-6.

In various embodiments, the therapeutic agent is a chimeric polypeptide comprising a sequence at least about 70%, 75%, 80%, 85%, 90%, 95%, or 100% identical to any one of SEQ ID NOS: 794-802.

Another aspect of the present disclosure is a method of treating a condition in a subject in need thereof. The method comprising administering to the subject any herein-disclosed device.

In some embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.

In embodiments, the method comprises spinal fusion. In the method, the spinal fusion may comprise posterior lumbar fusion (PLF) and/or interbody fusion.

In various embodiments, the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

Yet another aspect is a method for manufacturing a three-dimensional structure. The method comprises steps of providing a solution comprising a ceramic, a polymer, and optionally an anti-foaming agent and/or dispersing agent, mixing the solution to obtain an ink formulation, and depositing the ink formulation in a three-dimensional form. The method includes: (i) an ink formulation comprising about 30% to about 70% by weight ceramic and about 5% to about 60% by weight polymer, and/or (ii) a three-dimensional structure that comprises about 50% to about 100% by weight ceramic and about 0% to about 50% by weight polymer.

In some embodiments, the ceramic of the ink formulation and/or three-dimensional structure comprises calcium phosphate, hydroxyapatite, fluorapatite, bone, silicate, or vanadate, or a combination thereof.

In embodiments, the ceramic of the ink formulation and/or three-dimensional structure comprises beta-tricalcium phosphate (β-TCP).

In various embodiments, the polymer of the ink formulation comprises a first polymer comprising polycaprolactone and a second polymer comprising polyethylene glycol. In the method, the ink formulation may comprise about 10% to about 30% by weight polycaprolactone and about 10% to about 30% by weight polyethylene glycol.

The three-dimensional structure may comprise about 100% by weight ceramic.

In some embodiments, the three-dimensional structure comprises about 100% by weight beta-tricalcium phosphate (β-TCP).

In various embodiments, the three-dimensional structure comprises about 70% to about 80% by weight ceramic, and about 20% to about 30% by weight polymer.

The three-dimensional structure may comprise about 70% to about 80% by weight beta-tricalcium phosphate (β-TCP), and about 20% to about 30% by weight polycaprolactone.

In embodiments, the method further comprises combining the three-dimensional structure with a therapeutic agent. In the method, the therapeutic agent may comprise a mammalian growth factor or a functional portion thereof and/or one or more polypeptides selected from Table 4, or a functional portion thereof. The therapeutic agent may comprise a bone morphogenetic protein (BMP).

In some embodiments, the therapeutic agent comprises a targeting moiety that non-covalently binds to the three-dimensional structure. In the method, the targeting moiety may bind to the printed three-dimensional structure with an affinity of about 1 pM to about 100 μm.

The targeting moiety may comprise a polypeptide at least about 70%, 75%, 80%, 85%, 90%, 95%, or 100% identical to any one of the sequences of Tables 5-6.

In various embodiments, the targeting moiety comprises about 2, 3, 4, 5, 6, 7, 8, 9, or 10 sequences selected from the sequences of Tables 5-6.

In embodiments, the therapeutic agent is a chimeric polypeptide comprising a sequence at least about 70%, 75%, 80%, 85%, 90%, 95%, or 100% identical to any one of SEQ ID NOS: 794-802.

In an aspect, the present disclosure provides a method for treating a condition in a subject in need thereof. The method comprises a step of administering to the subject any herein-disclosed three-dimensional structure.

In some embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.

The method may comprise spinal fusion. In various embodiments, the spinal fusion comprises posterior lumbar fusion (PLF) and/or interbody fusion.

In embodiments, the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

In another aspect, the present disclosure provides an ink formulation for three-dimensional printing, the formulation comprising about 30% to about 70% by weight ceramic, and about 5% to about 30% by weight polymer.

In some embodiments, the ceramic comprises calcium phosphate, hydroxyapatite, fluorapatite, bone, silicate, or vanadate, or a combination thereof.

In embodiments, the ceramic comprises beta-tricalcium phosphate (β-TCP).

The formulation may comprise about 50% to about 70% by weight ceramic, about 10% to about 30% by weight a first polymer, and about 10% to about 30% by weight a second polymer or about 50% to about 70% by weight beta-tricalcium phosphate (β-TCP), about 10% to about 30% by weight a first polymer comprising polycaprolactone, and about 10% to about 30% by weight a second polymer comprising polyethylene glycol.

In various embodiments, the formulation comprises about 50% to about 70% by weight ceramic, about 5% to about 15% by weight polymer, and optionally an anti-foaming agent and/or a dispersing agent or comprising about 50% to about 70% by weight tricalcium phosphate, about 5% to about 15% by weight poloxamer, and optionally an anti-foaming agent and/or a dispersing agent. The formulation may comprise about 0.1% to about 1% by weight anti-foaming agent, in which the anti-foaming agent optionally comprises an alcohol and/or about 0.1% to about 1% by weight dispersing agent, in which the dispersing agent optionally comprises ammonium polyacrylate.

The formulation may comprise about 40% to about 60% by weight ceramic, about 5% to about 15% by weight polymer, and about 30% to about 40% by weight solvent or about 40% to about 60% by weight beta-tricalcium phosphate (β-TCP), about 5% to about 15% by weight polycaprolactone, and about 30% to about 40% by weight solvent. In embodiments, the solvent comprises dichloromethane, 2-butoxyethanol, dibutyl phthalate, or chloroform, or a combination thereof.

In a further aspect, the present disclosure provides a method for preparing a three-dimensional structure. The method comprises using any herein-disclosed formation as an ink in a three-dimensional printing method.

An aspect of the present disclosure is a three-dimensional structure prepared using any herein-disclosed formation.

In various embodiments, the three-dimensional structure comprises about 50% to about 100% by weight ceramic.

In some embodiments, the three-dimensional structure comprises about 50% to about 100% by weight tricalcium phosphate.

In embodiments, the three-dimensional structure comprises about 50% to about 90% by weight tricalcium phosphate and about 10% to about 50% polymer. The polymer may comprise polycaprolactone.

The three-dimensional structure may comprise one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

Another aspect of the present disclosure is a three-dimensional structure comprising about 50% to about 100% by weight ceramic, and about 0% to about 50% polymer.

In some embodiments, the ceramic comprises calcium phosphate, hydroxyapatite, fluorapatite, bone, silicate, or vanadate, or a combination thereof.

In embodiments, the ceramic comprises beta-tricalcium phosphate (β-TCP).

In some embodiments, the three-dimensional structure comprises about 50% to about 100% by weight ceramic.

In various embodiments, the three-dimensional structure comprises about 100% by weight ceramic.

In further embodiments, the three-dimensional structure comprises about 100% by weight tricalcium phosphate.

The three-dimensional structure may comprise about 50% to about 90% by weight ceramic and about 10% to about 50% polymer or about 50% to about 90% by weight tricalcium phosphate and about 10% to about 50% polymer. In embodiments, the polymer comprises polycaprolactone.

In embodiments, the three-dimensional structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

The three-dimensional structure may be prepared by three-dimensional printing methods.

A further aspect of the present disclosure is a method for delivering a therapeutic agent to a subject in need thereof. The method comprises delivering to an organ or tissue of the subject a device comprising a therapeutic agent and any-herein disclosed three-dimensional structure.

In an aspect the present disclosure provides a device comprising a therapeutic agent and any herein-disclosed three-dimensional structure.

In various embodiments, the therapeutic agent comprises a mammalian growth factor or functional portion thereof.

In embodiments, the therapeutic agent comprises one or more polypeptides selected from Table 4, or a functional portion thereof.

In some embodiments, the therapeutic agent comprises a bone morphogenetic protein (BMP).

The device may comprise a targeting moiety. In embodiments, the targeting moiety comprises a polypeptide comprising one or more sequences at least about 70%, 75%, 80%, 85%, 90%, 95%, or 100% identical to any one of the sequences of Tables 5-6. The targeting moiety may comprise about 2, 3, 4, 5, 6, 7, 8, 9, or 10 sequences selected from the sequences of Tables 5-6. In some embodiments, the targeting moiety non-covalently binds to the three-dimensional structure.

In various embodiments, the targeting moiety is connected to the therapeutic agent in a chimeric polypeptide.

In some embodiments, the chimeric polypeptide comprises a sequence at least about 70%, 75%, 80%, 85%, 90%, 95%, or 100% identical to any one of SEQ ID NOS: 794-802.

In another aspect, the present disclosure provides a method of preparing any herein-disclosed device. The method comprising combining the therapeutic agent and the three-dimensional structure, where the therapeutic agent non-covalently binds to the three-dimensional structure.

In yet another aspect, the present disclosure provides a method for treating a condition in a subject in need thereof. The method comprising administering to the subject any herein-disclosed three-dimensional structure or any herein-disclosed device.

In various embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof. The method may comprise spinal fusion, e.g., spinal fusion that comprises posterior lumbar fusion (PLF) and/or interbody fusion.

In some embodiments, the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

In a herein-disclosed aspect or embodiment, the three-dimensional structure may have a density of between about 1 g/cm³ and about 3 g/cm³.

In a herein-disclosed aspect or embodiment, the three-dimensional structure may have an open porosity of between about 15% and about 45%.

In a herein-disclosed aspect or embodiment, the three-dimensional structure may have a specific surface area of between about 0.50 m²/g and about 1.0 m²/g.

In a herein-disclosed aspect or embodiment, the three-dimensional structure may have a fiber diameter of about 325 μm and about 475 μm.

In a herein-disclosed aspect or embodiment, the three-dimensional structure may have a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

In an embodiment, a three-dimensional structure has a density of about 2.44 g/cm³, open porosity of about 19.6%, and a fiber diameter of about 384 μm.

In another embodiment, a three-dimensional structure has a density of about 1.32 g/cm³, open porosity of about 38%, and a fiber diameter of about 394 μm.

In yet another embodiment, a three-dimensional structure has a density of about 1.49 g/cm³, open porosity of about 31%, specific surface area of 0.81 m²/g, and a fiber diameter of 420 μm.

Advantages of the materials and methods described herein include providing a 3D-printed, customizable implant, as well as more universal strip, block, or cylindrical objects. As the implants are 3D-printed, precise control of the implant geometry is possible. Implants printed with these inks are thus suitable for many different therapies including long bone repair, spinal fusion, maxio-facial structures, etc. The ceramic content of the implantable devices can be loaded with tetherable growth factor to enhance regeneration of bone tissue. The different formulations of the inks produce materials that result in implantable devices with differing properties, including differing porosity and flexibility.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1A is a schematic of the process of fabricating a 3D printed object using a calcium phosphate ceramic ink.

FIG. 1B is a flow chart illustrating the steps for creating the calcium phosphate ceramic ink used in FIG. 1A.

FIG. 2A is a schematic of the process of fabricating a 3D printed object using a calcium phosphate-polymer ink.

FIG. 2B is a flow chart illustrating the steps for creating the calcium phosphate-polymer ink used in FIG. 2A.

FIG. 2C are micrographs of an example 3D printed object made with calcium phosphate-polymer ink as outlined in FIG. 2A.

FIG. 3A is a schematic of the process of fabricating a 3D printed object using a composite ink.

FIG. 3B is a flow chart illustrating the steps for creating the composite ink used in FIG. 3A.

FIG. 3C are micrographs of an example 3D printed object made with composite ink as outlined in FIG. 2A.

FIG. 4A is a picture of an example 3D printed object made with composite ink 320 as described herein.

FIG. 4B is a picture of the 3D printed object of FIG. 4A, prior to hydration.

FIG. 4C is a picture of the 3D printed object of FIG. 4A, hydrated with arterial blood.

FIG. 4D is a picture of the 3D printed object of FIG. 4C, ready for implantation.

Like reference symbols in the various drawings indicate like elements.

### DETAILED DESCRIPTION

Various calcium phosphate ceramic ink formulations for 3D printing of porous bone scaffold implants have been developed. The ink formulations possess a shear-thinning behavior that enables 3D printing via extrusion-based techniques. These inks are formulated by dispersing ceramic particles into the liquid ink components with an asymmetric centrifugal mixing process. One such ink 3D-prints a rigid calcium phosphate ceramic material, another a calcium phosphate-polymer composite material that is flexible and easily handled, and a third to 3D-print structures via melt extrusion that contain a blend of β-TCP powder, a non-water-soluble polymer and a water-soluble polymer that is porous and flexible.

The 3D-printed structures described herein are coated with a tetherable protein (for example, tBMP2) during fabrication that promotes bone growth. In some instances, the 3D-printed structures can be seeded with cells post-fabrication such that the cells occupy the pores of the 3D-printed structure. Once prepared, the 3D-printed structures can be surgically implanted into a patient for surgical bone replacement and grafting.

### Ink Formulations

In one aspect, provided herein are formulations for fabrication of 3D-printed structures. As a non-limiting example, the formulations include a ceramic material such as calcium phosphate (e.g., tricalcium phosphate, beta tricalcium phosphate, alpha tricalcium phosphate), hydroxyapatite, fluorapatite, bone (e.g., demineralized bone), glasses (bioglasses) such as silicates, vanadates, and related ceramic minerals, or chelated divalent metal ions, or a combination thereof. In some embodiments, the ceramic material comprises beta-tricalcium phosphate (β-TCP). In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic by weight of the formulation. For instance, the formulation is about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic by weight. In some embodiments, the ceramic is β-TCP. In some embodiments, the β-TCP is introduced into the formulation as a powder. In some embodiments, the formulation comprises one or more additional components. Non-limiting examples of additional components include: water, polymer, antifoaming agent, dispersing agent, solvent, and plasticizer.

In some embodiments, the formulation comprises one or more polymers, e.g., about 1, 2, 3, 4, or 5 polymers. Non-limiting examples of polymers include poly(ethylene oxide), poly(propylene oxide), polyethylene glycol (PEG), and polyester. In some embodiments, the formulation is about 5-30 percent polymer by weight. In some embodiments, the formulation is about 5-30, 5-25, 5-20, 5-15, 5-10, 10-30, 10-25, 10-20, 10-15, 15-30, 15-25, 15-20, 20-30, 20-25, or 25-30 percent by weight of a first polymer, and about 5-30 percent by weight of a second polymer. In an example, the polymer comprises a poloxamer. Poloxamers are block copolymers of poly(ethylene oxide) (PEO) and poly(propylene oxide) (PPO). A non-limiting example of a poloxamer is poloxamer 407, such as Pluronic® F-127. In some cases, the formulation comprises about 5-20, 5-15, 5-10, 10-20, 10-15, or 15-20 percent poloxamer 407 by weight. As another example, the polymer comprises polyethylene glycol (PEG). In some cases, the formulation comprises about 10-30, 10-25, 10-20, 10-15, 15-30, 15-25, 15-20, 20-30, 20-25, or 25-30 percent by weight PEG. In some cases, the formulation comprises PEG having a molecular weight of 1,500 g/mol. As another example, the polymer comprises a polyester. In some embodiments, the polyester is a biodegradable polyester such as polycaprolactone (PCL). In some cases, the formulation comprises about 10-30, 10-25, 10-20, 10-15, 15-30, 15-25, 15-20, 20-30, 20-25, or 25-30 percent by weight PCL. In some cases, the formulation comprises PCL having a molecular weight of 50,000 g/mol.

In some embodiments, the formulation comprises one or more antifoaming agents, e.g., about 1, 2, or 3 antifoaming agents. Non-limiting examples of antifoaming agents include 1-ocatanol, 2-butoxyethanol, oleic acid, sulfonated oils, organic phosphates, and dimethylpolysiloxane. In some embodiments, the antifoaming agent comprises an octanol, such as 1-octanol. In some cases, the formulation comprises about 0.25-0.75, 0.25-0.70, 0.25-0.65, 0.25-0.60, 0.25-0.55, 0.25-0.50, 0.25-0.45, 0.25-0.40, 0.25-0.35, 0.25-0.30, 0.30-0.75, 0.30-0.70, 0.30-0.65, 0.30-0.60, 0.30-0.55, 0.30-0.50, 0.30-0.45, 0.30-0.40, 0.30-0.35, 0.35-0.75, 0.35-0.70, 0.35-0.65, 0.35-0.60, 0.35-0.55, 0.35-0.50, 0.35-0.45, 0.35-0.40, 0.40-0.75, 0.40-0.70, 0.40-0.65, 0.40-0.60, 0.40-0.55, 0.40-0.50, 0.40-0.45, 0.45-0.75, 0.45-0.70, 0.45-0.65, 0.45-0.60, 0.45-0.55, 0.45-0.50, 0.50-0.75, 0.50-0.70, 0.50-0.65, 0.50-0.60, 0.50-0.55, 0.55-0.75, 0.55-0.70, 0.55-0.65, 0.55-0.60, 0.60-0.75, 0.60-0.70, 0.60-0.65, 0.65-0.75, or 0.65-0.70 percent 1-octanol by weight. In some embodiments, the antifoaming agent comprises 2-butoxyethanol. In some cases, the formulation comprises about 2.5-12.5, 2.5-10, 2.5-7.5, 2.5-5, 5-12.5, 5-10, 5-7.5, 7.5-12.5, 7.5-10, or 10-12.5 percent 2-butoxyethanol by weight.

In some embodiments, the formulation comprises one or more dispersing agents, e.g., about 1, 2, or 3 dispersing agents. Non-limiting examples of dispersing agents include Darvan® 821-A, Darvan® C-N, Darvan® 811, Darvan® 811D, Darvan® 7-N, and Darvan® 7-NS. In some cases, the formulation comprises about 0.1-0.3, 0.1-0.25, 0.1-0.2, 0.1-0.15, 0.15-0.3, 0.15-0.25, 0.15-0.2, 0.2-0.3, 0.2-0.25, or 0.25-0.3 percent dispersing agent by weight. In some embodiments, the dispersing agent comprises Darvan® 821-A.

In some embodiments, the formulation comprises one or more solvents, e.g., about 1, 2, or 3 solvents. In some cases, the formulation comprises about 25-35, 25-33, 25-31, 25-29, 25-27, 27-33, 27-31, 27-29, 29-35, 29-33, 29-31, 31-35, or 33-35 percent solvent by weight. Illustrative solvents include organochlorides such as dichloromethane (DCM) and chloroform; a solvent may be 2-butoxyethanol. In some cases, a solvent combination may comprise dibutyl phthalate.

In some embodiments, the formulation comprises one or more plasticizers, e.g., about 1, 2, or 3 plasticizers. In some cases, the formulation comprises about 2-6, 2-5, 2-4, 2-3, 3-6, 3-5, 3-4, 4-6, or 5-6 percent plasticizer by weight. In some embodiments, the plasticizer comprises a phthalate. Non-limiting example phthalates include dibutyl phthalate (DBP), benzyl butyl phthalate (BBP), and diethylhexyl-phthalate (DEHP).

In some embodiments, a formulation comprises β-TCP and a polymer. Polymers include PEO, PPO, PEG, or polyester, or a combination thereof. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent β-TCP by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% β-TCP by weight. In some cases, the formulation comprises about 5-20, 5-15, 5-10, 10-20, 10-15, or 15-20 percent poloxamer 407 by weight. In some cases, the formulation comprises about 10-30, 10-25, 10-20, 10-15, 15-30, 15-25, 15-20, 20-30, 20-25, or 25-30 percent by weight PEG. In some cases, the formulation comprises about 10-30, 10-25, 10-20, 10-15, 15-30, 15-25, 15-20, 20-30, 20-25, or 25-30 percent by weight PCL. In some embodiments, the formulation further comprises an antifoaming agent. In some embodiments, the formulation further comprises a dispersing agent. In some embodiments, the formulation further comprises a solvent. In some embodiments, the formulation further comprises a plasticizer.

In some embodiments, a formulation comprises β-TCP and an antifoaming agent. The antifoaming agent may comprise 1-octanol and/or 2-butoxyethanol. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent β-TCP by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% β-TCP by weight. In some cases, the formulation comprises about 0.25-0.75, 0.25-0.70, 0.25-0.65, 0.25-0.60, 0.25-0.55, 0.25-0.50, 0.25-0.45, 0.25-0.40, 0.25-0.35, 0.25-0.30, 0.30-0.75, 0.30-0.70, 0.30-0.65, 0.30-0.60, 0.30-0.55, 0.30-0.50, 0.30-0.45, 0.30-0.40, 0.30-0.35, 0.35-0.75, 0.35-0.70, 0.35-0.65, 0.35-0.60, 0.35-0.55, 0.35-0.50, 0.35-0.45, 0.35-0.40, 0.40-0.75, 0.40-0.70, 0.40-0.65, 0.40-0.60, 0.40-0.55, 0.40-0.50, 0.40-0.45, 0.45-0.75, 0.45-0.70, 0.45-0.65, 0.45-0.60, 0.45-0.55, 0.45-0.50, 0.50-0.75, 0.50-0.70, 0.50-0.65, 0.50-0.60, 0.50-0.55, 0.55-0.75, 0.55-0.70, 0.55-0.65, 0.55-0.60, 0.60-0.75, 0.60-0.70, 0.60-0.65, 0.65-0.75, or 0.65-0.70 percent 1-octanol by weight. In some cases, the formulation comprises about 2.5-12.5, 2.5-10, 2.5-7.5, 2.5-5, 5-12.5, 5-10, 5-7.5, 7.5-12.5, 7.5-10, or 10-12.5 percent 2-butoxyethanol by weight. In some embodiments, the formulation further comprises a polymer. In some embodiments, the formulation further comprises a dispersing agent. In some embodiments, the formulation further comprises a solvent. In some embodiments, the formulation further comprises a plasticizer.

In some embodiments, a formulation comprises β-TCP and a dispersing agent. Non-limiting examples of dispersing agents include Darvan® 821-A, Darvan® C-N, Darvan® 811, Darvan® 811D, Darvan® 7-N, and Darvan® 7-NS. The dispersing agent may comprise Darvan® 821-A. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent β-TCP by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% β-TCP by weight. In some cases, the formulation comprises about 0.1-0.3, 0.1-0.25, 0.1-0.2, 0.1-0.15, 0.15-0.3, 0.15-0.25, 0.15-0.2, 0.2-0.3, 0.2-0.25, or 0.25-0.3 percent dispersing agent by weight. In some embodiments, the formulation further comprises a polymer. In some embodiments, the formulation further comprises a solvent. In some embodiments, the formulation further comprises a plasticizer. In some embodiments, the formulation further comprises an antifoaming agent.

In some embodiments, a formulation comprises β-TCP and a solvent. The solvent may comprise an organochloride such as dichloromethane (DCM) and chloroform; a solvent may be 2-butoxyethanol. A solvent combination may comprise dibutyl phthalate. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent β-TCP by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% β-TCP by weight. In some cases, the formulation comprises about 25-35, 25-33, 25-31, 25-29, 25-27, 27-33, 27-31, 27-29, 29-35, 29-33, 29-31, 31-35, or 33-35 percent solvent by weight. In some embodiments, the formulation further comprises a polymer. In some embodiments, the formulation further comprises a dispersing agent. In some embodiments, the formulation further comprises a plasticizer. In some embodiments, the formulation further comprises an antifoaming agent.

In some embodiments, a formulation comprises β-TCP and a plasticizer. The solvent may comprise a phthalate. Non-limiting example phthalates include dibutyl phthalate (DBP), benzyl butyl phthalate (BBP), and diethylhexyl-phthalate (DEIP). In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent β-TCP by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% β-TCP by weight. In some cases, the formulation comprises about 25-35, 25-33, 25-31, 25-29, 25-27, 27-33, 27-31, 27-29, 29-35, 29-33, 29-31, 31-35, or 33-35 percent solvent by weight. In some cases, the formulation comprises about 2-6, 2-5, 2-4, 2-3, 3-6, 3-5, 3-4, 4-6, or 5-6 percent plasticizer by weight. In some embodiments, the formulation further comprises a polymer. In some embodiments, the formulation further comprises a dispersing agent. In some embodiments, the formulation further comprises a solvent. In some embodiments, the formulation further comprises an antifoaming agent.

In one aspect, a formulation comprises about 45% to about 65% β-TCP. For instance, the formulation comprises about 45-60, 45-55, 45-50, 50-65, 50-60, 50-55, 55-65, 55-60, 60-65, 45, 46, 47, 48, 49, 50, 51, 52, 53, 54, 55, 56, 57, 58, 59, 60, 61, 62, 63, 64, or 65 percent β-TCP by weight. In some embodiments, the formulation comprises water, e.g., about 20-40, 20-35, 20-30, 20-25, 25-40, 25-35, 25-30, 30-40, 30-35, or 35-40 percent water by weight. In some embodiments, the formulation comprises a polymer, e.g., about 5-20, 5-15, 5-10, 10-20, 10-15, or 15-20 percent polymer by weight. The polymer may be a poloxamer, such as poloxamer 407. In some embodiments, the formulation comprises an antifoaming agent, e.g., about 0.25-0.75, 0.25-0.70, 0.25-0.65, 0.25-0.60, 0.25-0.55, 0.25-0.50, 0.25-0.45, 0.25-0.40, 0.25-0.35, 0.25-0.30, 0.30-0.75, 0.30-0.70, 0.30-0.65, 0.30-0.60, 0.30-0.55, 0.30-0.50, 0.30-0.45, 0.30-0.40, 0.30-0.35, 0.35-0.75, 0.35-0.70, 0.35-0.65, 0.35-0.60, 0.35-0.55, 0.35-0.50, 0.35-0.45, 0.35-0.40, 0.40-0.75, 0.40-0.70, 0.40-0.65, 0.40-0.60, 0.40-0.55, 0.40-0.50, 0.40-0.45, 0.45-0.75, 0.45-0.70, 0.45-0.65, 0.45-0.60, 0.45-0.55, 0.45-0.50, 0.50-0.75, 0.50-0.70, 0.50-0.65, 0.50-0.60, 0.50-0.55, 0.55-0.75, 0.55-0.70, 0.55-0.65, 0.55-0.60, 0.60-0.75, 0.60-0.70, 0.60-0.65, 0.65-0.75, or 0.65-0.70 percent antifoaming agent by weight. The antifoaming agent may be 1-octanol. In some embodiments, the formulation comprises a dispersing agent, e.g., about 0.1-0.3, 0.1-0.25, 0.1-0.2, 0.1-0.15, 0.15-0.3, 0.15-0.25, 0.15-0.2, 0.2-0.3, 0.2-0.25, or 0.25-0.3 percent dispersing agent. The dispersing agent may be Darvan 821-A. In a non-limiting embodiment, the formulation comprises about 45-65% by weight ceramic, about 20-40% by weight deionized water, about 5-20% by weight polymer, about 0.25-0.75% antifoaming agent, and about 0.1-0.3% dispersing agent. For example, the formulation may comprise about 45-65% by weight β-TCP powder, about 20-40% by weight deionized water, about 5-20% by weight poloxamer 407, about 0.25-0.75% 1-octanol, and about 0.1-0.3% Darvan 821-A.

In another aspect, a formulation comprises about 30% to about 50% β-TCP. For instance, the formulation comprises about 30-50, 30-45, 30-40, 30-35, 35-50, 35-45, 35-40, 40-50, 40-45, 45-50, 30, 31, 32, 33, 34, 35, 36, 37, 38, 39, 40, 41, 42, 43, 44, 45, 46, 47, 48, 49, or 50 percent β-TCP by weight. In some embodiments, the formulation comprises a polymer, e.g., about 10-20, 10-18, 10-16, 10-14, 10-12, 12-20, 12-18, 12-16, 12-14, 14-20, 14-18, 14-16, or 16-18 percent polymer by weight. The polymer may be a polyester such as polycaprolactone (PCL). In some embodiments, the formulation comprises a solvent, e.g., about 25-35, 25-33, 25-31, 25-29, 25-27, 27-33, 27-31, 27-29, 29-35, 29-33, 29-31, 31-35, or 33-35 percent solvent by weight. The solvent may be an organochloride such as dichloromethane (DCM) and chloroform; a solvent may be 2-butoxyethanol. In some cases, a solvent combination may comprise dibutyl phthalate. In some embodiments, the formulation comprises an antifoaming agent, e.g., about 2.5-12.5, 2.5-10, 2.5-7.5, 2.5-5, 5-12.5, 5-10, 5-7.5, 7.5-12.5, 7.5-10, or 10-12.5 percent antifoaming agent by weight. The antifoaming agent may be 2-butoxyethanol. In some embodiments, the formulation comprises a plasticizer, e.g., about 2-6, 2-5, 2-4, 2-3, 3-6, 3-5, 3-4, 4-6, or 5-6 percent plasticizer by weight. The plasticizer may comprise a phthalate such as dibutyl phthalate (DBP), benzyl butyl phthalate (BBP), or diethylhexyl-phthalate (DEHP). In a non-limiting embodiment, the formulation comprises about 30-50% by weight ceramic, about 10-20% by weight polymer, about 25-35% by weight solvent, about 2.5-12.5% by weight antifoaming agent, and about 2-6% by weight plasticizer. For example, the formulation may comprise about 30-50% by weight β-TCP powder, about 10-20% by weight PCL, about 25-35% by weight dichloromethane, about 2.5-12.5% by weight 2-butoxyethanol, and about 2-6% by weight dibutyl phthalate.

In another aspect, a formulation comprises about 30% to about 70% β-TCP. For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight β-TCP. In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be polycaprolactone (PCL). In some embodiments, the formulation comprises a second polymer, e.g., about 10-30, 10-25, 10-20, 10-15, 15-30, 15-25, 15-20, 20-30, 20-25, or 25-30 percent by weight second polymer. The second polymer may be polyethylene glycol (PEG). In a non-limiting embodiment, the formulation comprises about 30-70% by weight ceramic, about 10-30% by weight a first polymer, and about 10-30% by weight a second polymer. For example, the formulation may comprise about 30-70% by weight β-TCP, about 10-30% by weight PCL, and about 10-30% by weight PEG.

### 3D Printed Structures

In another aspect, provided herein are 3D printed structures. The structures may be prepared using an ink formulation and/or method of manufacture described herein.

In some embodiments, the structure comprises a ceramic material such as a calcium phosphate. In some embodiments, the structure comprises about 50-100, 50-95, 50-90, 50-85, 50-80, 50-75, 50-70, 50-65, 50-60, 50-55, 55-100, 55-95, 55-90, 55-85, 55-80, 55-75, 55-70, 55-65, 55-60, 60-100, 60-95, 60-90, 60-85, 60-80, 60-75, 60-70, 60-65, 65-100, 65-95, 65-90, 65-85, 65-80, 65-75, 65-70, 70-100, 70-95, 70-90, 70-85, 70-80, 70-75, 75-100, 75-95, 75-90, 75-85, 75-80, 80-100, 80-95, 80-90, 80-85, 85-100, 85-95, 85-90, 90-100, 90-95, 95-100, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent ceramic material. In some cases, the ceramic material is calcium phosphate, such as beta-tricalcium phosphate (β-TCP).

In a non-limiting example, a structure has about 90-100% ceramic material such as β-TCP. In some cases, the structure has about 90, 91, 92, 93, 94, 95, 96, 97, 98, 99, or 100% ceramic material such as β-TCP. In some embodiments, the structure has about 0-10% polymer such as Pluronic F-127. In some cases, the structure has about 0, 1, 2, 3, 4, 5, 6, 7, 8, 9, or 10% polymer such as Pluronic F-127. Example structures include those having: 100% ceramic (e.g., β-TCP), about 100% (e.g., β-TCP), about 99% ceramic (e.g., β-TCP) and about 1% polymer (e.g., Pluronic F-127) by weight, about 98% ceramic (e.g., β-TCP) and about 2% polymer (e.g., Pluronic F-127) by weight, about 97% ceramic (e.g., β-TCP) and about 3% polymer (e.g., Pluronic F-127) by weight, about 96% ceramic (e.g., β-TCP) and about 4% polymer (e.g., Pluronic F-127) by weight, about 95% ceramic (e.g., β-TCP) and about 5% polymer (e.g., Pluronic F-127) by weight, about 94% ceramic (e.g., β-TCP) and about 6% polymer (e.g., Pluronic F-127) by weight, about 93% ceramic (e.g., β-TCP) and about 7% polymer (e.g., Pluronic F-127) by weight, about 92% ceramic (e.g., β-TCP) and about 8% polymer (e.g., Pluronic F-127) by weight, about 91% ceramic (e.g., β-TCP) and about 9% polymer (e.g., Pluronic F-127) by weight, and about 90% ceramic (e.g., β-TCP) and about 10% polymer (e.g., Pluronic F-127) by weight. In an example embodiment, the structure is about 100% β-TCP.

In some embodiments a three-dimensional structure has a density of between about 1 g/cm³ and about 3 g/cm³ (e.g., about 1, 1.1, 1.15, 1.2, 1.25, 1.3, 1.35, 1.4, 1.45, 1.5, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, or 3 g/cm³ or any value therebetween). In some embodiments a three-dimensional structure has an open porosity of between about 15% and about 45% (e.g., 15, 20, 25, 30, 35, 40%, or 45%, or any value therebetween). In some embodiments a three-dimensional structure has a specific surface area of between about 0.50 m²/g and about 1.0 m²/g (e.g., 0.5, 0.6, 0.7, 0.8, 0.9, or 1.0 m²/g, or any value therebetween). In some embodiments a three-dimensional structure has a fiber diameter of about 325 μm and about 475 μm (e.g., 325, 350, 375, 400, 425, 450, or 475 μm, or any value therebetween).

In some embodiments a three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

The structure may be manufactured using 3D printing from an ink comprising about 45-65% by weight ceramic, about 20-40% by weight deionized water, about 5-20% by weight polymer, about 0.25-0.75% antifoaming agent, and about 0.1-0.3% dispersing agent. For example, the structure is manufactured from an ink comprising about 45-65% by weight β-TCP powder, about 20-40% by weight deionized water, about 5-20% by weight poloxamer 407, about 0.25-0.75% 1-octanol, and about 0.1-0.3% Darvan 821-A. An example 3D printing method using a (3-TCP and Pluronic F-127 ink is provided in the manufacture methods herein. In some embodiments, the structure is about 100% β-TCP.

In some embodiments, the structure has a density of about 2.44 g/cm³, open porosity of about 19.6%, and a fiber diameter of about 384 μm.

In another non-limiting example, a structure has about 50-90% ceramic material such as β-TCP. In some cases, the structure has about 50, 55, 60, 65, 70, 75, 80, 85, or 90% ceramic material such as β-TCP. In some embodiments, the structure has about 10-50% polymer such as polycaprolactone (PCL). In some cases, the structure has about 10, 15, 20, 25, 30, 35, 40, 45, or 50% polymer such as PCL. Example structures include those having: about 85-90% ceramic (e.g., β-TCP) and about 10-15% polymer (e.g., PCL) by weight, about 80-85% ceramic (e.g., β-TCP) and about 15-20% polymer (e.g., PCL) by weight, about 75-80% ceramic (e.g., β-TCP) and about 20-25% polymer (e.g., PCL) by weight, about 70-75% ceramic (e.g., β-TCP) and about 25-30% polymer (e.g., PCL) by weight, about 65-70% ceramic (e.g., β-TCP) and about 30-35% polymer (e.g., PCL) by weight, about 60-65% ceramic (e.g., β-TCP) and about 35-40% polymer (e.g., PCL) by weight, about 55-60% ceramic (e.g., β-TCP) and about 40-45% polymer (e.g., PCL) by weight, about 50-55% ceramic (e.g., β-TCP) and about 45-50% polymer (e.g., PCL) by weight, about 90% ceramic (e.g., β-TCP) and about 10% polymer (e.g., PCL) by weight, about 89% ceramic (e.g., 0-TCP) and about 11% polymer (e.g., PCL) by weight, about 88% ceramic (e.g., β-TCP) and about 12% polymer (e.g., PCL) by weight, about 87% ceramic (e.g., β-TCP) and about 13% polymer (e.g., PCL) by weight, about 86% ceramic (e.g., β-TCP) and about 14% polymer (e.g., PCL) by weight, about 85% ceramic (e.g., β-TCP) and about 15% polymer (e.g., PCL) by weight, about 84% ceramic (e.g., β-TCP) and about 16% polymer (e.g., PCL) by weight, about 83% ceramic (e.g., β-TCP) and about 17% polymer (e.g., PCL) by weight, about 82% ceramic (e.g., β-TCP) and about 18% polymer (e.g., PCL) by weight, about 81% ceramic (e.g., β-TCP) and about 19% polymer (e.g., PCL) by weight, about 80% ceramic (e.g., β-TCP) and about 20% polymer (e.g., PCL) by weight, about 79% ceramic (e.g., β-TCP) and about 21% polymer (e.g., PCL) by weight, about 78% ceramic (e.g., β-TCP) and about 22% polymer (e.g., PCL) by weight, about 77% ceramic (e.g., β-TCP) and about 23% polymer (e.g., PCL) by weight, about 76% ceramic (e.g., 0-TCP) and about 24% polymer (e.g., PCL) by weight, about 75% ceramic (e.g., β-TCP) and about 25% polymer (e.g., PCL) by weight, about 74% ceramic (e.g., β-TCP) and about 26% polymer (e.g., PCL) by weight, about 73% ceramic (e.g., β-TCP) and about 27% polymer (e.g., PCL) by weight, about 72% ceramic (e.g., β-TCP) and about 28% polymer (e.g., PCL) by weight, about 71% ceramic (e.g., β-TCP) and about 29% polymer (e.g., PCL) by weight, about 70% ceramic (e.g., β-TCP) and about 30% polymer (e.g., PCL) by weight, about 69% ceramic (e.g., β-TCP) and about 31% polymer (e.g., PCL) by weight, about 68% ceramic (e.g., β-TCP) and about 32% polymer (e.g., PCL) by weight, about 67% ceramic (e.g., β-TCP) and about 33% polymer (e.g., PCL) by weight, about 66% ceramic (e.g., β-TCP) and about 34% polymer (e.g., PCL) by weight, about 65% ceramic (e.g., β-TCP) and about 35% polymer (e.g., PCL) by weight, about 64% ceramic (e.g., β-TCP) and about 36% polymer (e.g., PCL) by weight, about 63% ceramic (e.g., 0-TCP) and about 37% polymer (e.g., PCL) by weight, about 62% ceramic (e.g., β-TCP) and about 38% polymer (e.g., PCL) by weight, about 61% ceramic (e.g., β-TCP) and about 39% polymer (e.g., PCL) by weight, about 60% ceramic (e.g., β-TCP) and about 40% polymer (e.g., PCL) by weight, about 59% ceramic (e.g., β-TCP) and about 41% polymer (e.g., PCL) by weight, about 58% ceramic (e.g., β-TCP) and about 42% polymer (e.g., PCL) by weight, about 57% ceramic (e.g., β-TCP) and about 43% polymer (e.g., PCL) by weight, about 56% ceramic (e.g., β-TCP) and about 44% polymer (e.g., PCL) by weight, about 55% ceramic (e.g., β-TCP) and about 45% polymer (e.g., PCL) by weight, about 54% ceramic (e.g., β-TCP) and about 46% polymer (e.g., PCL) by weight, about 53% ceramic (e.g., β-TCP) and about 47% polymer (e.g., PCL) by weight, about 52% ceramic (e.g., β-TCP) and about 48% polymer (e.g., PCL) by weight, about 51% ceramic (e.g., β-TCP) and about 49% polymer (e.g., PCL) by weight, and about 50% ceramic (e.g., β-TCP) and about 50% polymer (e.g., PCL) by weight.

The structure may be manufactured using 3D printing from an ink comprising about 30-50% by weight β-TCP powder, about 10-20% by weight polymer, about 25-35% by weight solvent, about 2.5-12.5% by weight antifoaming agent, and about 2-6% by weight plasticizer. For example, the structure may be manufactured from an ink comprising about 30-50% by weight β-TCP powder, about 10-20% by weight PCL, about 25-35% by weight dichloromethane, about 2.5-12.5% by weight 2-butoxyethanol, and about 2-6% by weight dibutyl phthalate. The structure may be manufactured using 3D printing from an ink comprising about 30-70% by weight ceramic, about 10-30% by weight a first polymer, and about 10-30% by weight a second polymer. For example, the structure may be manufactured from an ink comprising about 30-70% by weight β-TCP, about 10-30% by weight PCL, and about 10-30% by weight PEG.

In some embodiments, the structure has a density of about 1.32 g/cm³, open porosity of about 38%, and a fiber diameter of about 394 μm.

In some embodiments, the structure has a density of about 1.49 g/cm³, open porosity of about 31%, specific surface area of 0.81 m²/g, and a fiber diameter of about 420 μm.

In some embodiments, the structure has an elastic modulus (stiffness) of about 100-150, 100-140, 100-130, 100-120, 100-110, 110-150, 110-140, 110-130, 110-120, 120-150, 120-140, 120-130, 130-150, 130-140, 140-150, 100, 115, 120, 125, 130, 135, 140, 145, or 150 MPa. In some embodiments, the compositions of ink formulations herein are varied to optimize specific surface area. The surface area may be optimized for combination with a certain therapeutic agent. For example, the structure has a surface area of about 0.2-2 m²/g for combination with a BMP protein (e.g., tBMP-2). In some embodiments, the surface area of a structure herein is about 0.2-2, 0.2-1.8, 0.2-1.6, 0.2-1.4, 0.2-1.2, 0.2-1, 0.2-0.8, 0.2-0.6, 0.2-0.4, 0.4-2, 0.4-1.8, 0.4-1.6, 0.4-1.4, 0.4-1.2, 0.4-1, 0.4-0.8, 0.4-0.6, 0.6-2, 0.6-1.8, 0.6-1.6, 0.6-1.4, 0.6-1.2, 0.6-1, 0.6-0.8, 0.8-2, 0.8-1.8, 0.8-1.6, 0.8-1.4, 0.8-1.2, 0.8-1, 1-2, 1-1.8, 1-1.6, 1-1.4, 1-1.2, 1.2-2, 1.2-1.8, 1.2-1.6, 1.2-1.4, 1.4-2, 1.4-1.8, 1.4-1.6, 1.6-2, 1.6-1.8, or 1.8-2 m²/g. In some embodiments, the surface area is calculated by Brunauer-Emmett-Teller (BET) by gas physisorption.

### Methods of Manufacture

In another aspect, provided are methods of manufacturing a structure using 3D printing techniques.

### 1) β-Tricalcium Phosphate Ceramic Ink

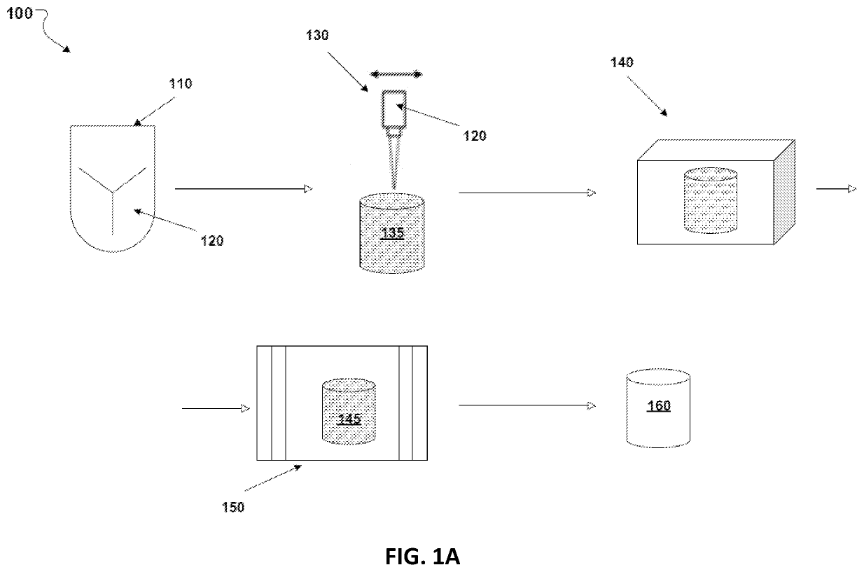

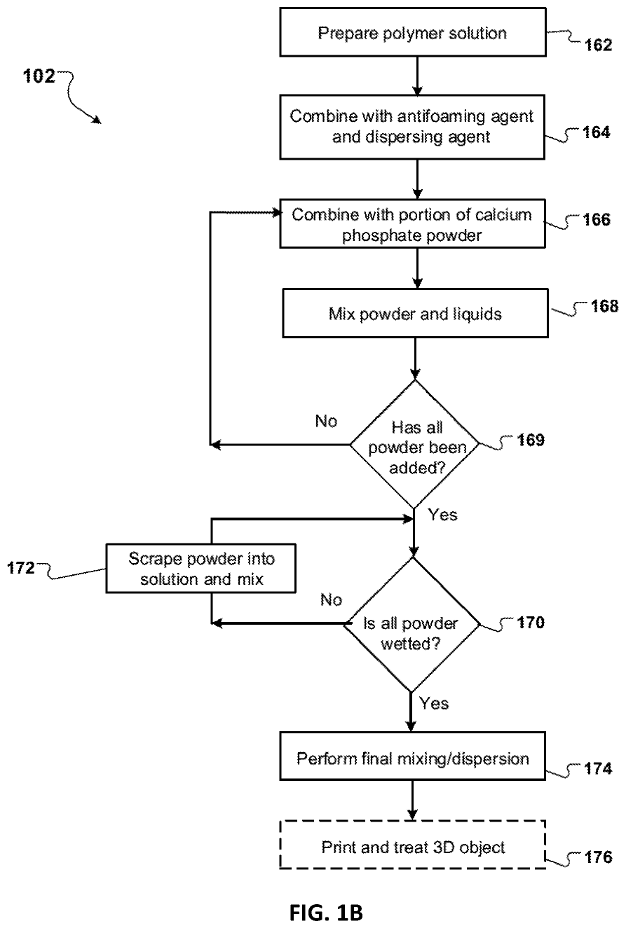

Referring to FIG. 1A, example process 100 is described for preparing a rigid calcium phosphate ceramic implantable structure 160. The implantable structure 160 is formed from a calcium phosphate ceramic ink 120 that is a mixture of a calcium phosphate ceramic powder, a water-soluble polymer binder, water, a dispersant, and an anti-foaming agent. A structure 135 printed with the calcium phosphate ceramic ink 120 is first dried, and then thermally processed to pyrolyze the water-soluble polymer binder and densify the remaining ceramic material to approximately 80% theoretical density or higher by high temperature sintering.

The process 100 includes a mixing step for preparing the extrudable calcium phosphate ceramic ink 120, a 3D printing step in which the prepared calcium phosphate ceramic ink 120 is fed through a 3D printer 130 to create a printed structure 135, a drying step where the printed structure 135 is placed in a drier set-up 140, and a final heat treatment step in which the dried scaffold or green body 145 is placed in a heater 150 to remove the water soluble polymer binder and sinter the calcium phosphate ceramic powder to form the final implantable structure 160.

This calcium phosphate ceramic ink 120 is powder-filled and includes a Pluronic® F-127 hydrogel material (Sigma-Aldrich, Missouri) that serves both as a gelling agent and a polymeric binder material for the ceramic green body 145 (after drying). The Pluronic® F-127 hydrogel is liquid at 4° C. and transitions to a gel material at room temperature. The gel properties of the Pluronic® F-127 hydrogel enable filaments extruded by the 3D printer 130 to maintain their shape during the printing process (which is done at room temperature). An example formulation of the extrudable calcium phosphate ceramic ink 120 is described in Table 1.

TABLE 1

| | Wt % | Component | |
|---|---|---|---|
| Component | in ink | characteristics | Purpose |
| β-TCP Powder | 62.1 | Milled powder, 3-5 | Sinterable β-TCP powder, densifies |
| | | micron particle size | to ~80% theoretical β-TCP density |
| | | | after sintering |
| Deionized water | 28.1 | N/A | Solvent for Pluronic ® F-127 polymer |
| Pluronic ® F-127 | 9.1 | Product is a fine | Provides gelation of ink during room |
| polymer | | powder | temperature 3D printing; after drying, |
| | | | the remaining polymer binds ceramic |
| | | | particles in a green ceramic body |
| 1-Octanol | 0.5 | clear liquid | Antifoaming agent to help minimize |
| | | | trapped air bubbles in ink |
| Darvan ® 821-A | 0.2 | 40% Ammonium | Dispersing agent that helps break down |
| | | polyacrylate (3500 | β-TCP powder agglomerates during |
| | | molecular weight) | mixing and stabilize dispersion after |
| | | dissolved in water | mixing |

Referring as well to FIG. 1B, an example method 102 is illustrated for creating the calcium phosphate ceramic ink 120. First, a polymeric solution is prepared (step 162) using a Pluronic® F-127 solution. Preparing the Pluronic® F-127 solution involves first preparing a stock 24.5% Pluronic® F-127 solution by placing 37.75 g of deionized water in a container (e.g., a 75-cc polypropylene container) and chilling it by partial submersion in an ice bath. The ice bath is transferred to a stir plate 110 (illustrated in FIG. 1A), and a magnetic stir bar added to the container to vigorously mix the contents. 12.25 g of Pluronic® F-127 powder is added to the 37.75 g of deionized water with constant stirring on the stir plate 110. The mixture is stirred for approximately 1 hour or until the Pluronic® F-127 powder is fully dissolved.

To make a 10-cc batch of the calcium phosphate ceramic ink 120, 6.438 g of the cold 24.5% Pluronic® F-127 solution that has been prepared is then combined with a dispersing agent and an antifoaming agent (e.g., in a 30 cc capped syringe), step 164. In this example method 102, 0.033 g of Darvan® 821-A (Vanderbilt Minerals, Connecticut) and 0.082 g of 1-octanol are added to the 24.5% Pluronic® F-127 solution. This combination of Pluronic® F-127 solution, Darvan® 821-A, and 1-octanol is then homogenized, for example, in a FlackTek Speedmixer at 3500 rpm for 1 min.

The mixed solution is then combined with the calcium phosphate powder, in this case β-TCP powder. This addition of the powder is done step-wise; for example, 10.745 g of the β-TCP powder is divided into three batches. The first ⅓ of the powder is added to the solution, step 166, and mixed at 2500 rpm for 1 min to wet the powder, step 168. These steps are repeated with the second ⅓ of powder being added (repeating step 166), and again mixed at 2500 rpm for 1 min (repeating step 168), and repeated again with the final ⅓ of the powder and mixing at 2500 rpm for 1 min. If any powder remains unwetted, determined at step 170, the sides of the container containing the solution and powder are scraped down with a spatula to incorporate the dry powder into the wet solution and the ink mixture mixed at 3500 rpm for 1 min, step 172. The ink mixture is again assessed as to whether the powder has been sufficiently wetted (step 170), and the scraping step and mixing step are repeated as necessary. When all the powder has been wetted, a final mixing/dispersion step is performed by a final mixing at 3500 rpm for 5 min, step 174. The prepared calcium phosphate ceramic ink 120 is now ready to use in 3D printing and the 3D printed structures 165 can be further processed.

Referring back to FIG. 1A, the prepared calcium phosphate ceramic ink 120 is now ready for use in the printer 130. For example, the calcium phosphate ceramic ink 120 can be used for printing with an Allevi 2 Bioprinter (Allevi, Pennsylvania) that uses a 10-cc syringe. In some examples, the calcium phosphate ceramic ink 120 can be printed with a 400-micron inner diameter conical metallic Luer lock tip using 90 psi pressure and 14 mm/s tip velocity. In other examples, the ink 120 can be printed with 625-micron inner diameter conical metallic Luer lock tip using 70 psi pressure and 14 mm/s tip velocity. The printed structures 135 can be printed on Teflon plumber's tape applied to a smooth glass surface as the build platform, such as a glass microscope slide or larger glass plate.

Once the 3D printing process is complete, the printed structure 135 is placed in the drier set-up 140. For example, this can be a plastic box with a loosely fitting lid in which the printed structure 135 is left overnight at room temperature to allow for evaporation of water from the 3D-printed calcium phosphate ceramic ink 120 forming the printed structure 135. The evaporation results in a stiff ceramic green body 145 in which the β-TCP powder is bound together by dry Pluronic® F-127 polymer.

The green body 145 is then heat treated in a heater 150 with a combined binder burnout/sintering heat treatment. This treatment first removes the Pluronic® F-127 polymeric binder, and then the remaining ceramic β-TCP powder is sintered. For example, the green body 145 is placed in the heater 150 that is a 1200° C. maximum temperature muffle furnace located inside a fume hood.

The first binder-burnout portion of the heat treatment involves a temperature ramp from room temperature to 600° C. at a heating rate of 1° C./min, followed by a hold for 1 hour at 600° C. The sintering portion of the heat treatment subsequently involves a ramp from 600° C. to 1140° C. at a rate of 5° C./min, followed by a hold at 1140° C. for 4 hrs. The burned-out and sintered body is then cooled, with a cooldown ramp from 1140° C. to room temperature at a rate of 5° C./min. The result is an implantable structure 160 with a density of approximately 2.44 g/cc, or 79.4% of the theoretical density of β-TCP (measured using Archimedes method).

The calcium phosphate ceramic ink 120 is described as being formulated with β-TCP ceramic powder, but in some embodiments, the powder filler could also be other bone regenerative materials such as hydroxyapatite or bioglass (e.g., Combeite 45S5 Bioactive Glass), other ceramics, or demineralized bone matrix. The drying and thermal processing steps (e.g., drying conditions, binder burnout heating schedule, sintering heat schedule and max temperature) would be altered appropriately based on each material.

### 2) β-Tricalcium Phosphate Polycaprolactone Composite Ink

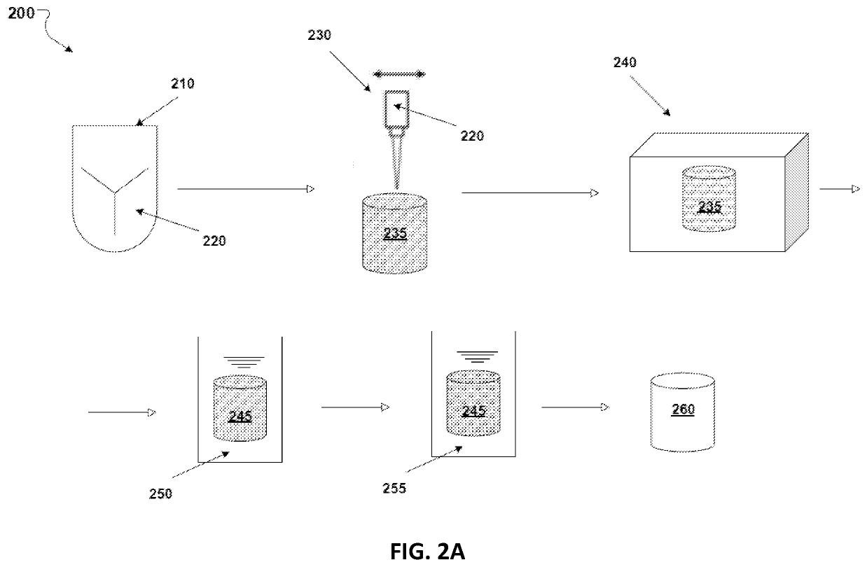

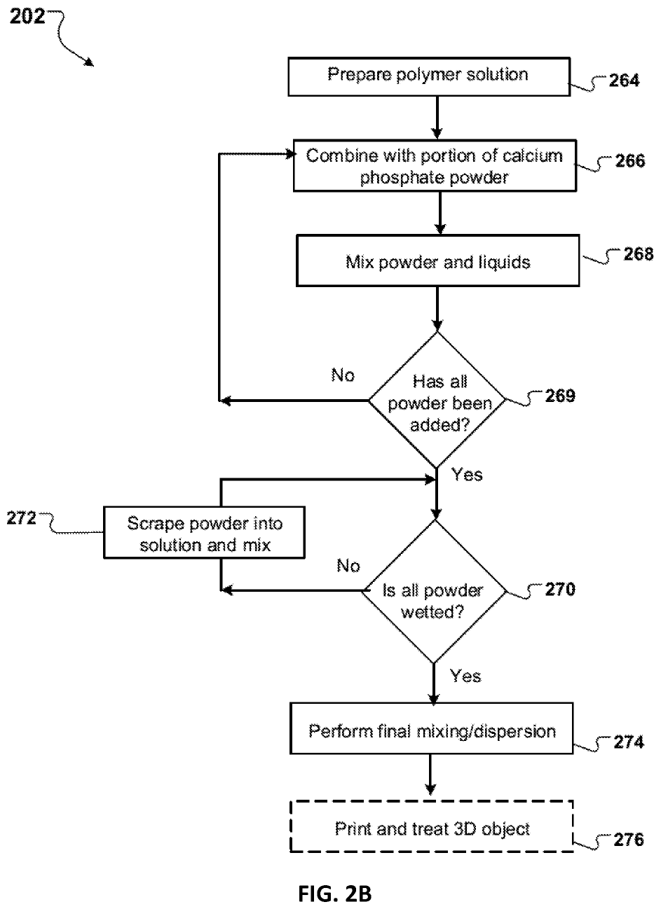

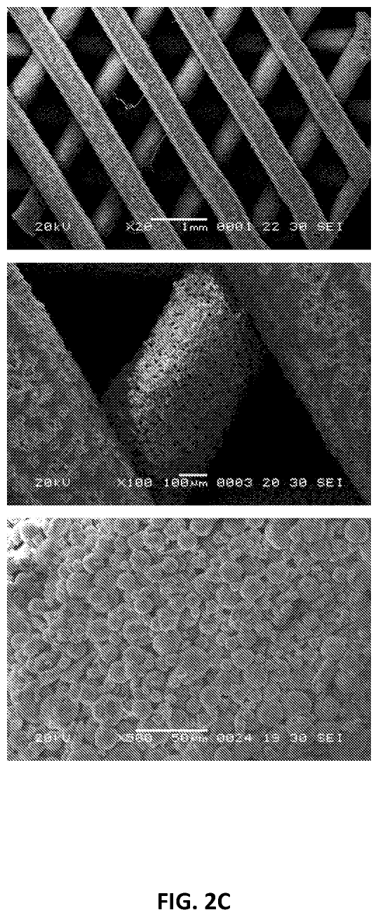

Referring to FIG. 2A, in another embodiment, a calcium phosphate-polymer ink 220 results in a printed structure that is flexible, porous, and easily handled by a user (e.g., a surgeon). The calcium phosphate-polymer ink 220 contains a mixture of calcium phosphate ceramic powder, polymer binder (polycaprolactone or PCL in this instance) and three solvents with different vapor pressures (at room temperature). The illustrative process 200 illustrated in FIG. 2A includes a mixing step for creating the extrudable calcium phosphate-polymer ink 220. The prepared phosphate-polymer ink 220 is then printed in a 3D printer 230 to form the printed structures 235. The printed structures 235 are dried to evaporate the high volatility solvent and form a dried body 245 as the dissolved polymer material condenses, binding the ceramic particles of the printed structures 235 together while imparting flexibility. The remaining low volatility solvents are removed from the dried body 245 by successive soaking in an ethanol/water mixture 250 followed by soaking in water 255. Fabrication of the final calcium phosphate-polymer composite implantable structure 260 does not require thermal processing after 3D printing. An example formulation of the extrudable calcium phosphate-polymer ink 220 is described in Table 2.

TABLE 2

| | Wt % | Component | |
|---|---|---|---|
| Component | in ink | characteristics | Purpose |
| β-TCP Powder | 47.1 | Spray-dried powder, 10 | Spherical β-TCP Powder for good |
| | | to 30-micron particle size | extrudability during 3D printing |
| Polycaprolactone | 11.8 | 50,000 MW, fine powder | Flexible polymer binder for β-TCP |
| (PCL) | | | powder |
| Dichloromethane | 29.9 | High volatility solvent, | Fast evaporation of this solvent |
| | | clear liquid | enables quick solidification of |
| | | | extruded filament during 3D |
| | | | printing |
| 2-butoxyethanol | 7.5 | Medium volatility | Medium volatility solvent that slows |
| | | solvent, clear liquid | precipitation of PCL during drying, |
| | | | facilitates fusing to subsequent |
| | | | adjacent filaments during 3D |
| | | | printing |
| Dibutyl phthalate | 3.7 | Low volatility solvent, | Low volatility solvent that slows |
| | | clear liquid | precipitation of PCL during drying, |
| | | | facilitates fusing to subsequent |
| | | | adjacent filaments during 3D |
| | | | printing |

The trisolvent blend with varied vapor pressures enables initial hardening of the printed filaments of the calcium phosphate-polymer ink 220 that are extruded from the printer 230, as the high volatility dichloromethane evaporates first. The two lower volatility solvents (2-butoxyethanol and dibutyl phthalate) slow the precipitation of the dissolved PCL binder, allowing it to coat the β-TCP powder and neck between adjacent particles while also creating an interconnected porous network. Additionally, the lower volatility solvents remain in the printed structure 235 for some time, which facilitates fusing of a printed filament to adjacent 3D-printed filaments (either beside of or on top of the previously extruded filament).

Referring as well to FIG. 2B, an example method 102 is illustrated for preparing the calcium phosphate-polymer ink 220. To prepare a 10.2 cc batch of the calcium phosphate-polymer ink 220, first a polymer solution is prepared (step 264). This is a PCL solution, formed by combining the PCL and the solvents in a container 210. First, 5.09 g of dichloromethane is mixed with 1.27 g of 2-butoxyethanol, then 0.64 g of dibutyl phthalate is added as is 2 g of PCL powder. The solution is mixed to dissolve the PCL in the solvent blend, for example, in a FlackTek Speedmixer at 3500 rpm for 5 min.

Calcium phosphate powder (β-TCP spray-dried powder) is then added, step 266. This addition is performed step-wise, with ˜5 g of the β-TCP spray-dried powder added to the PCL solution, which is then mixed at 2500 rpm for 2 minutes to wet the powder, step 268. ˜2 g of the β-TCP spray-dried powder is added (returning to step 266) and mixed at 2500 rpm for 2 minutes to wet the powder (returning to step 268). A final ˜1 g of the β-TCP spray-dried powder is added to the mixture, and mixed again at 2500 rpm for 2 minutes to wet the powder.

Once all the β-TCP spray-dried powder has been added, it is determined if all the powder has been wetted, step 270. If not, the sides of the container are scraped down with a spatula to incorporate the dry powder into the wet solution and mixed at 3500 rpm for 1 min, step 272. The scraping step and mixing step are repeated as necessary. When all the powder has been wetted, a final mixing/dispersion step is performed by a final mixing at 3500 rpm for 5 min, step 274. The prepared calcium phosphate-polymer ink 220 is now ready to use in 3D printing and further processing of the 3D printed structures 265, step 276.

Referring back to FIG. 2A, the prepared calcium phosphate-polymer ink 220 is then ready for use in a 3D printer 230. For example, the calcium phosphate-polymer ink 220 can be used for printing with an Allevi 2 Bioprinter that extrudes the calcium phosphate-polymer ink 220 from a 10-cc syringe. The calcium phosphate-polymer ink 220 can be printed with 400-micron inner diameter conical metallic Luer lock tip using 20-25 psi pressure and 15 mm/s tip velocity.

The printed structures 235 can be printed on painter's tape applied to a smooth glass surface as a build platform, such as a glass microscope slide or larger glass plate, or on to 2 mm thick silicone gasket material. The 3D-printed structures 235 are allowed to dry in place on the build platform of the printer 230 for approximately 15-30 min. The printed structures 235 will at this point detach naturally from the build platform of the printer 230 and can be handled.

To remove any residual solvents in the printed structures 235, they are washed in in the ethanol/water mixture 250 (e.g., 70% ethanol for 30 min), followed by soaking in water 255 (e.g., two subsequent 30 min washes in deionized water).

The calcium phosphate-polymer ink 220 can also be formulated with other bone regenerative powder materials such as hydroxyapatite, bioglass, other ceramics, or demineralized bone matrix.

FIG. 2C shows scanning electron microscope (SEM) micrographs of a printed structure 235 fabricated with the prepared calcium phosphate-polymer ink 220. The printed structure 235 in this example is a lattice structure and is shown at three magnifications (20×, 100×, and 500×).

### 3) β-Tricalcium Phosphate Polycaprolactone Composite Ink

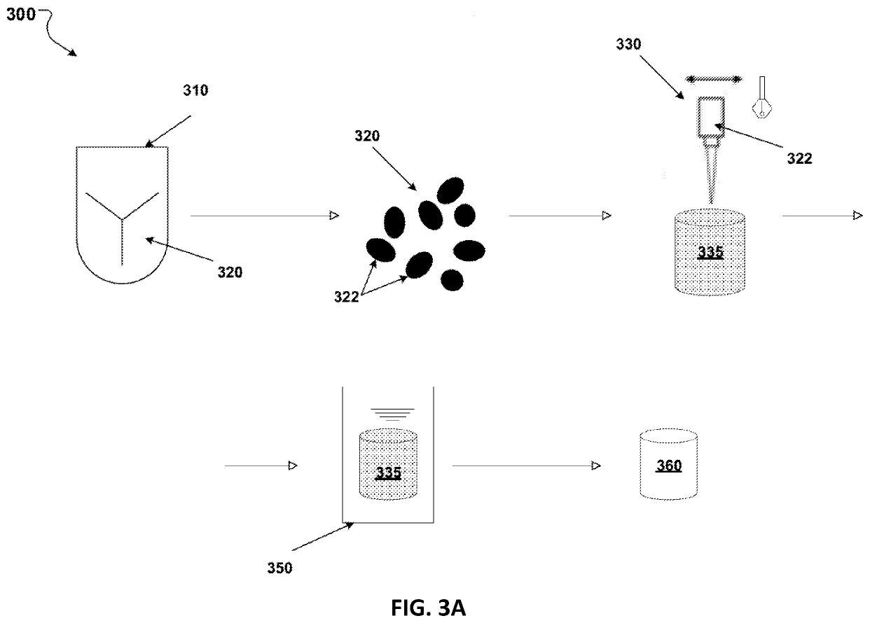

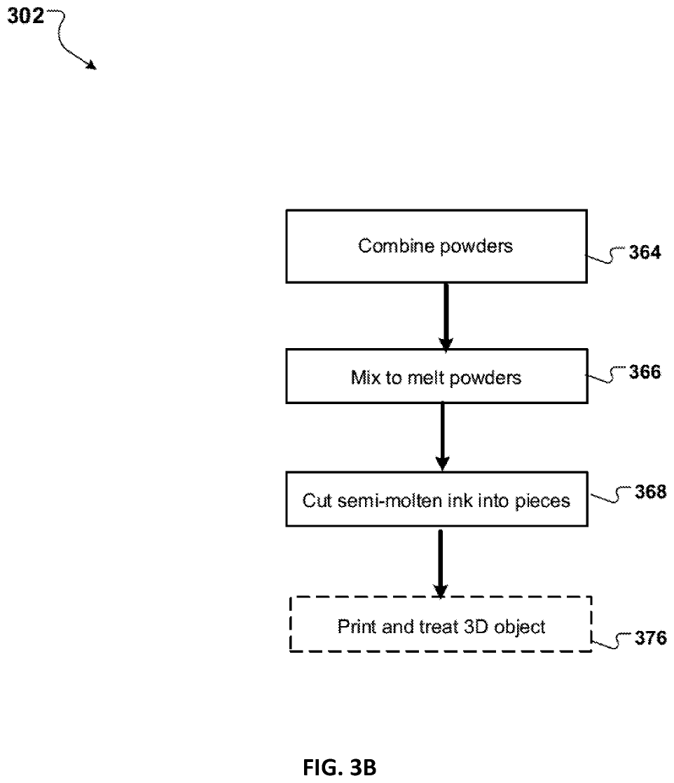

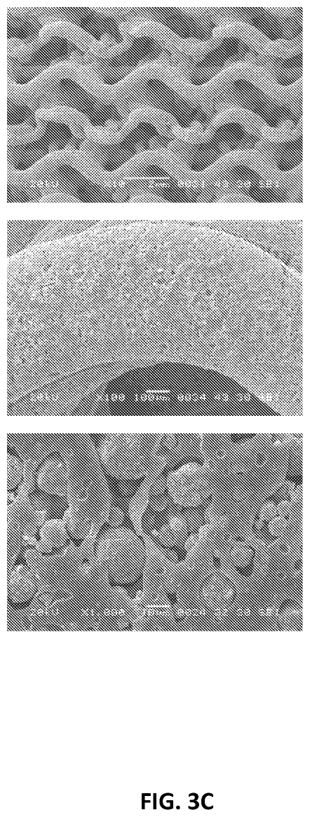

Referring to FIG. 3A, a composite ink 320 is used to 3D print porous, flexible implantable structures 360 (via melt extrusion) that contain a blend of β-TCP powder, a non-water-soluble polymer (PCL), and a water-soluble polymer (polytheylene glycol). After printing with a 3D printer 330, the water-soluble polymer component is dissolved out of the material, resulting in a porous structure that exposes the β-TCP powder previously trapped in the interior of the 3D-printed filaments. Flexibility of the 3D-printed structures is also enhanced after leeching out the water-soluble component. A sample formulation of the extrudable composite ink 220 is described in Table 3.

TABLE 3

| | Wt % | Component | |
|---|---|---|---|
| Component | in ink | characteristics | Purpose |
| β-TCP Powder | 60 | Spray-dried powder, 10 | Spherical β-TCP Powder for good |
| | | to 38-micron particle | extrudability during 3D printing |
| | | size | |
| Polycaprolactone | 20 | 50,000 MW, fine | Flexible polymer binder for β-TCP |
| | | powder, Tmelt = 60° C. | powder; not water soluble |
| Polyethylene | 20 | 1,500 MW, flake, | Water soluble polymer with low |
| Glycol | | Tmelt = 60° C. | melt viscosity for good extrusion |

Referring as well to FIG. 3B, to make a 3.2 cc batch of the composite ink 320 the various powders are combined, step 364. 2.062 g of β-TCP powder, 2.062 g of PCL powder, and 0.884 g of polyethylene glycol flake are added to a container 310 (e.g., a glass jar). The container 310 is placed in a mixer and the powders mixed, for example, in a FlackTek Speedmixer at 300 rpm for 2 min at 300 rpm to homogenize the powder blend. Higher rpm mixing is then carried out for an additional 5 min at 3500 rpm to melt the powders, step 366. During this mixing, the internal friction causes the PCL and polyethylene glycol to melt, changing the powders to a viscous molten liquid. This liquid phase mixing facilitates intimate dispersion of the β-TCP powder into the molten polymer blend. Thus, while the composite ink 320 is still molten and viscous, it can be roughly molded with spatulas or cut into pieces 322 that fit within the printer syringe 330, step 368. For example, the ink can be cut into ˜1 cm³ sized pieces. This size of solid PCL/β-TCP chunks can fit into a syringe for melt-extrusion printing, for example, using an Allevi 2 Bioprinter that extrudes the composite ink 320 from a 10-cc stainless steel syringe. This can be a syringe with a 400-micron inner diameter conical metallic Luer lock tip using 90 psi pressure and 5 mm/s-10 mm/s tip velocity.

The composite ink 320 is then largely ready for 3D printing of the printed structure 335 and further treatment, step 376. Prior to printing with the printer 330, the extruder chamber (e.g., the stainless-steel syringe) is heated to ensure melting of the composite ink 320. The ink can be heated to 100° C.-110° C. and allowed to dwell for 1 hour. The melted ink can then create the printed structure 335 on painter's tape applied to a smooth glass surface, such as a glass microscope slide or larger glass plate.

The printed structure 335 is then soaked overnight (or longer, depending on the size of the printed object) in distilled water 350 to dissolve the polyethylene glycol from the printed material, creating a porous and flexible β-TCP/PCL composite implantable structure 360.

The composite ink 320 can be formulated with other bone regenerative powder materials such as hydroxyapatite or bioglass, or other ceramics or even demineralized bone matrix. It can also be formulated with other biocompatible polymers, such as poly(lactic-co-glycolic acid), poly(lactic acid), or poly(L-lactide-co-caprolactone).

FIG. 3C shows SEM micrographs of a printed structure 335 fabricated with the prepared composite ink 320. The printed structure 335 in this example is a lattice structure and is shown at three magnifications (20×, 100×, and 1000×).

Any of the 3D-printed implantable structures 160, 260, 360 described herein can then be coated with a tetherable protein (for example, tBMP2) as part of the treatment of the 3D implantable structures (steps 176, 276, 376). Following completion of the implantable structures using any of the methods discussed above, the implantable structures can then be washed in an acidic sodium acetate buffer. This can be one, two, or more washes. The washing can then be followed by a two-hour incubation of the 3D-implantable structures in sodium acetate buffer that contains a 1 mg/mL concentration of tBMP2 protein. The tetherable tBMP2 binds to the β-TCP surface of the implantable structures in a monolayer.

In some embodiments, a bone putty (rather than a 3D-printed component) is used to deliver tetherable tBMP2 to a bone regeneration site. A putty material can be roughly shaped by hand into the shape and size of the bone void and inserted into the cavity where bone regeneration is desired. A sodium carboxymethylcellulose (CMC) hydrogel can be mixed with β-TCP granules that have been coated with tBMP2 to create a bone void-filling putty. A putty of 50 wt % β-TCP (coated with tBMP2) and 50 wt % sodium CMC hydrogel can be formulated with asymmetric centrifugal mixing. To make a 6 wt % sodium CMC hydrogel, 10.5 g of deionized water is placed in a polypropylene container and 0.9 g of sodium CMC powder is added to the water. The material is mixed in a FlackTek Speedmixer at 3500 rpm for 10-11 minutes to fully dissolve the sodium carboxymethylcellulose powder and the resulting material is an extremely viscous gel. After that, 3.6 g of additional deionized water is added to dilute the thick gel and mixed at 3500 rpm for 2 minutes. Fifteen grams of β-TCP granules (250 μm-1000 μm, previously coated with tBMP2) are then added in stepwise increments (e.g., 5 g, 5 g, 5 g) followed by mixing at 2500 rpm for 1 min after each granule addition. A final mixing step of 3500 rpm for 2 min is done to fully homogenize the putty (asymmetric centrifugal mixing). The β-TCP particles are coated with tBMP2 before mixing the β-TCP granules into the hydrogel.

In further embodiments, the ink formulations discussed herein can include a light-sensitive resin that is mixed with the ceramic powder for digital light processing (DLP), an additive manufacturing technique that is faster than robocasting or melt extrusion. Components in a photosensitive, ceramic-filled resin for DLP 3D printing of bone implants typically include ceramic powder (e.g., β-TCP, hydroxyapatite, bioglass, typically <10 μm particle size), one or more crosslinking acrylates or methacrylates (e.g., polyethylene glycol diacrylate, polycaprolactone methacrylate), a plasticizer to reduce resin viscosity (e.g., water), a dispersant to promote breakdown of powder agglomerates (e.g., Darvan® 821-A), photoinitiator to initiate the photocrosslinking reaction (e.g., Lithium phenyl-2,4,6-trimethylbenzoylphosphinate), and a photoabsorber to retain high x-y resolution (e.g., tartrazine). Once resin formulations are prepared by asymmetric centrifugal mixing of the components, the ink is exposed layer by layer to a DLP image, causing the lighted pixels to selectively solidify when the resin encounters the light. Once the implantable structure has been built up layer by layer, it can be thermally processed to burn out the included polymer and densify the ceramic (e.g., a polyetheylene glycol diacrylate-containing resin), or left as-is, resulting in a flexible ceramic/polymer composite implant (e.g., a polycaprolactone methacrylate-containing resin).

### Devices

In another aspect, provided are devices and kits comprising a 3D printed structure described herein and a therapeutic agent. In some embodiments, a device comprises the therapeutic agent connected to, dispersed within, or otherwise combined with the 3D printed structure. As used herein, a therapeutic agent is inclusive of a plurality of therapeutic agents, such as 2, 3, 4, or 5 therapeutic agents.

Therapeutic Agents

In some embodiments, the therapeutic agent comprises a mammalian growth factor or a functional portion thereof. Mammalian growth factors can be osteoinductive molecules that are capable of initiating and enhancing the bone repair process. A functional portion of the mammalian growth factor is a region that has a therapeutic effect. For instance, a functional portion of a mammalian growth factor is osteoinductive. As another example, a functional portion of a mammalian growth factor is capable of initiating and/or enhancing bone repair. A functional portion of a mammalian growth factor may have osteogenic activity.

Non-limiting examples of mammalian growth factors are described herein. In some instances, the mammalian growth factor comprises: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12), bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), and collagen binding protein (CBP), fibroblast growth factor 18 (FGF-18), keratinocyte growth factor (KGF), tumor necrosis factor alpha (TNFα), tumor necrosis factor (TNF)-related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), or wnt family member 16 (WNT16), or a mature peptide or functional portion thereof.

In some embodiments, the mammalian growth factor is a human growth factor. Non-limiting examples of human growth factors and mature peptides and/or functional portions thereof are provided in Table 4. In some embodiments, the mammalian growth factor comprises a sequence that is at least 70% identical (e.g., at least 75% identical, at least 80% identical, at least 85% identical, at least 90% identical, at least 95% identical, or at least 99% identical) to any of the sequences in Table 4 or any secreted human growth factor, and has osteogenic activity. In some embodiments, the amino acids in a mammalian growth factor that are conserved between different species are likely important for osteogenic activity and may not be mutated, while amino acids in a mammalian growth factor that are not conserved between different species are not likely important for osteogenic activity and may be mutated.

In some embodiments, the mammalian growth factor comprises BMP-2. In some embodiments, the mammalian growth factor is a mature peptide of BMP-2 (e.g., does not comprise a signal sequence). In some embodiments, the mammalian growth factor comprises a functional portion of BMP-2. In some embodiments, the functional portion of BMP-2 comprises a sequence at least about 70%, 75%, 80%, 85%, 90%, 95%, or 100% identical to: QAKHKQRKRLKSSCKRHPLYVDFSDVGWNDWIVAPPGYHAFYCHGECPFPLADHLNS TNHAIVQTLVNSVNSKIPKACCVPTELSAISMLYLDENEKVVLKNYQDMVVEGCGCR (SEQ ID NO: 454). In some embodiments, the mammalian growth factor comprises a sequence at least about 70%, 75%, 80%, 85%, 90%, 95%, or 100% identical to SEQ ID NO: 454. In some embodiments, the mammalian growth factor comprises a sequence at least about 90% identical to SEQ ID NO: 454. In some embodiments, the mammalian growth factor comprises SEQ ID NO: 454.

In some embodiments, the mammalian growth factor is a non-human mammalian growth factor. The non-human mammalian growth factor may be homologous to a human growth factor, such as one or more of the human growth factors of Table 4. In some embodiments, a non-human mammalian growth factor is homologous to a human growth factor if the non-human mammalian growth factor is at least about 80% identical to the human mammalian growth factor as determined using the NCBI Blast alignment algorithm as of the date of this filing. In some cases, the coverage is at least about 90%. In some embodiments, a non-human mammalian growth factor is homologous to a human growth factor if the non-human mammalian growth factor is at least about 80% positive as compared to the human mammalian growth factor as determined using the NCBI Blast alignment algorithm as of the date of this filing. In some cases, the coverage is at least about 90%. In some embodiments, a non-human mammalian growth factor is homologous to a human growth factor if the non-human mammalian growth factor aligned with the human growth factor using the NCBI Blast as of the date of this filing has an E value of less than about 1E-40, at least about 1E-50, 1E-60, 1E-70, or 1E-10, with a query cover of at least about 90%.

TABLE 4
Therapeutic Growth Factors

| | | SEQ ID |
|---|---|---|
| Name | Protein Sequence | NO: |
| EGF | NSDSECPLSHDGYCLHDGVCMYIEALDKYACNCVVGYIGERCQYRDLK | 442 |
| | WWELR | |
| PDGF | EEAEIPREVIERLARSQIHSIRDLQRLLEIDSVGSEDSLDTSLRAHGVHATK | 443 |
| | HVPEKRPLPIRRKR | |
| IGF-1 | GPETLCGAELVDALQFVCGDRGFYFNKPTGYGSSSRRAPQTGIVDECCFR | 444 |
| | SCDLRRLEMYCAPLKPAKSA | |
| FGF | FNLPPGNYKKPKLLYCSNGGHFLRILPDGTVDGTRDRSDQHIQLQLSAES | 445 |
| | VGEVYIKSIETGQYLAMDTDGLLYGSQTPNEECLFLERLEENHYNTYISK | |
| | KHAEKNWFVGLKKNGSCKRGPRTHYGQKAILFLPLPVSSD | |
| FGF2 | PALPEDGGSGAFPPGHFKDPKRLYCKNGGFFLRIHPDGRVDGVREKSDPH | 446 |
| | IKLQLQAEERGVVSIKGVCANRYLAMKEDGRLLASKCVTDECFFFERLES | |
| | NNYNTYRSRKYTSWYVALKRTGQYKLGSKTGPGQKAILFLPMSAKS | |
| FGF18 | EENVDFRIHVENQTRARDDVSRKQLRLYQLYSRTSGKHIQVLGRRISARG | 447 |
| | EDGDKYAQLLVETDTFGSQVRIKGKETEFYLCMNRKGKLVGKPDGTSKE | |
| | CVFIEKVLENNYTALMSAKYSGWYVGFTKKGRPRKGPKTRENQQDVHF | |
| | MKRYPKGQPELQKPFKYTTVTKRSRRIRPTHPA | |
| TGF-α | ENSTSPLSADPPVAAAVVSHFNDCPDSHTQFCFHGTCRFLVQEDKPACVC | 448 |
| | HSGYVGARCEHADLLAVVAASQKKQAITALVVVSIVALAVLIITCVLIHC | |
| | CQVRKHCEWCRALICRHEKPSALLKGRTACCHSETVV | |
| TGF-α | VVSHFNDCPDSHTQFCFHGTCRFLVQEDKPACVCHSGYVGARCEHADLL | 449 |
| | A | |
| TGF-β1 | ALDTNYCFSSTEKNCCVRQLYIDFRKDLGWKWIHEPKGYHANFCLGPCP | 450 |
| | YIWSLDTQYSKVLALYNQHNPGASAAPCCVPQALEPLPIVYYVGRKPKV | |
| | EQLSNMIVRSCKCS | |
| TGF-β3 | ALDTNYCFRNLEENCCVRPLYIDFRQDLGWKWVHEPKGYYANFCSGPCP | 451 |
| | YLRSADTTHSTVLGLYNTLNPEASASPCCVPQDLEPLTILYYVGRTPKVE | |
| | QLSNMVVKSCKCS | |
| OP-2 (BMP- | AVRPLRRRQPKKSNELPQANRLPGIFDDVHGSHGRQVCRRHELYVSFQD | 452 |
| 8) | LGWLDWVIAPQGYSAYYCEGECSFPLDSCMNATNHAILQSLVHLMMPD | |
| | AVPKACCAPTKLSATSVLYYDSSNNVILRKHRNMVVKACGCH | |
| BMP8A | AVRPLRRRQPKKSNELPQANRLPGIFDDVRGSHGRQVCRRHELYVSFQD | 453 |
| | LGWLDWVIAPQGYSAYYCEGECSFPLDSCMNATNHAILQSLVHLMKPN | |
| | AVPKACCAPTKLSATSVLYYDSSNNVILRKHRNMVVKACGCH | |
| BMP-2 | QAKHKQRKRLKSSCKRHPLYVDFSDVGWNDWIVAPPGYHAFYCHGECP | 454 |
| | FPLADHLNSTNHAIVQTLVNSVNSKIPKACCVPTELSAISMLYLDENEKV | |
| | VLKNYQDMVVEGCGCR | |
| BMP-3 | QWIEPRNCARRYLKVDFADIGWSEWIISPKSFDAYYCSGACQFPMPKSLK | 455 |
| | PSNHATIQSIVRAVGVVPGIPEPCCVPEKMSSLSILFFDENKNVVLKVYPN | |
| | MTVESCACR | |
| BMP-4 | SPKHHSQRARKKNKNCRRHSLYVDFSDVGWNDWIVAPPGYQAFYCHGD | 456 |
| | CPFPLADHLNSTNHAIVQTLVNSVNSSIPKACCVPTELSAISMLYLDEYDK | |
| | VVLKNYQEMVVEGCGCR | |
| BMP-5 | AANKRKNQNRNKSSSHQDSSRMSSVGDYNTSEQKQACKKHELYVSFRD | 457 |
| | LGWQDWIIAPEGYAAFYCDGECSFPLNAHMNATNHAIVQTLVHLMFPD | |
| | HVPKPCCAPTKLNAISVLYFDDSSNVILKKYRNMVVRSCGCH | |
| BMP- | SASSRRRQQSRNRSTQSQDVARVSSASDYNSSELKTACRKHELYVSFQDL | 458 |
| 6/VGR | GWQDWIIAPKGYAANYCDGECSFPLNAHMNATNHAIVQTLVHLMNPEY | |
| | VPKPCCAPTKLNAISVLYFDDNSNVILKKYRNMVVRACGCH | |
| BMP-7/OP- | STGSKQRSQNRSKTPKNQEALRMANVAENSSSDQRQACKKHELYVSFRD | 459 |
| 1 | LGWQDWIIAPEGYAAYYCEGECAFPLNSYMNATNHAIVQTLVHFINPET | |
| | VPKPCCAPTQLNAISVLYFDDSSNVILKKYRNMVVRACGCH | |
| BMP-9 | SAGAGSHCQKTSLRVNFEDIGWDSWIIAPKEYEAYECKGGCFFPLADDVT | 460 |
| | PTKHAIVQTLVHLKFPTKVGKACCVPTKLSPISVLYKDDMGVPTLKYHY | |
| | EGMSVAECGCR | |
| BMP-10 | NAKGNYCKRTPLYIDFKEIGWDSWIIAPPGYEAYECRGVCNYPLAEHLTP | 461 |
| | TKHAIIQALVHLKNSQKASKACCVPTKLEPISILYLDKGVVTYKFKYEGM | |
| | AVSECGCR | |
| BMP- | NLGLDCDEHSSESRCCRYPLTVDFEAFGWDWIIAPKRYKANYCSGQCEY | 462 |
| 11/GDF-11 | MFMQKYPHTHLVQQANPRGSAGPCCTPTKMSPINMLYFNDKQQIIYGKI | |
| | PGMVVDRCGCS | |
| BMP-12 | TALAGTRTAQGSGGGAGRGHGRRGRSRCSRKPLHVDFKELGWDDWIIA | 463 |
| | PLDYEAYHCEGLCDFPLRSHLEPTNHAIIQTLLNSMAPDAAPASCCVPAR | |
| | LSPISILYIDAANNVVYKQYEDMVVEACGCR | |
| BMP- | TAFASRHGKRHGKKSRLRCSKKPLHVNFKELGWDDWIIAPLEYEAYHCE | 464 |
| 13/GDF-6 | GVCDFPLRSHLEPTNHAIIQTLMNSMDPGSTPPSCCVPTKLTPISILYIDAG | |
| | NNVVYKQYEDMVVESCGCR | |
| BMP-15 | QADGISAEVTASSSKHSGPENNQCSLHPFQISFRQLGWDHWIIAPPFYTPN | 465 |
| | YCKGTCLRVLRDGLNSPNHAIIQNLINQLVDQSVPRPSCVPYKYVPISVL | |
| | MIEANGSILYKEYEGMIAESCTCR | |
| DPP isoform | IPVPQSKPLERHVEKSMNLHLLARSNVSVQDELNASGTIKESGVLVHEGD | 466 |
| 1 | RGRQENTQDGHKGEGNGSKWAEVGGKSFSTYSTLANEEGNIEGWNGDT | |
| | GKAETYGHDGIHGKEENITANGIQGQVSIIDNAGATNRSNTNGNTDKNTQ | |
| | NGDVGDAGHNEDVAVVQEDGPQVAGSNNSTDNEDEIIENSCRNEGNTSE | |
| | ITPQINSKRNGTKEAEVTPGTGEDAGLDNSDGSPSGNGADEDEDEGSGDD | |
| | EDEEAGNGKDSSNNSKGQEGQDHGKEDDHDSSIGQNSDSKEYYDPEGKE | |
| | DPHNEVDGDKTSKSEENSAGIPEDNGSQRIEDTQKLNHRESKRVENRITK | |
| | ESETHAVGKSQDKGIEIKGPSSGNRNITKEVGKGNEGKEDKGQHGMILG | |
| | KGNVKTQGEVVNIEGPGQKSEPGNKVGHSNTGSDSNSDGYDSYDFDDKS | |
| | MQG | |
| DPP isoform | IPVPQSKPLERHVEKSMNLHLLARSNVSVQDELNASGTIKESGVLVHEGD | 467 |
| 2 | RGRQENTQDGHKGEGNGSKWAEVGGKSFSTYSTLANEEGNIEGWNGDT | |
| | GKAETYGHDGIHGKEENITANGIQGQVSIIDNAGATNRSNTNGNTDKNTQ | |
| | NGDVGDAGHNEDVAVVQEDGPQVAGSNNSTDNEDEIIENSCRNEGNTSE | |
| | ITPQINSKRNGTKEAEVTPGTGEDAGLDNSDGSPSGNGADEDEDEGSGDD | |
| | EDEEAGNGKDSSNNSKGQEGQDHGKEDDHDSSIGQNSDSKEYYDPEGKE | |
| | DPHNEVDGDKTSKSEENSAGIPEDNGSQRIEDTQKLNHRESKRVENRITK | |
| | ESETHAVGKSQDKGIEIKGPSSGNRNITKEVGKGNEGKEDKGQHGMILG | |
| | KGNVKTQGEVVNIEGPGQKSEPGNKVGHSNTGSDSNSDGYDSYDFDDKS | |
| | MQGDDPNSSDESNGNDDANSESDNNSSSRGDASYNSDESKDNGNGSDS | |
| | KGAEDDDSDSTSDTNNSDSNGNGNNGNDDNDKSDSGKGKSDSSDSDSS | |
| | DSSNSSDSSDSSDSDSSDSNSSSDSDSSDSDSSDSSDSDSSDSSNSSDSSDSS | |
| | DSSDSSDSSDSSDSKSDSSKSESDSSDSDSKSDSSDSNSSDSSDNSDSSDSS | |
| | NSSNSSDSSDSSDSSDSSSSSDSSNSSDSSDSSDSSNSSESSDSSDSSDSDSS | |
| | DSSDSSNSNSSDSDSSNSSDSSDSSNSSDSSDSSDSSNSSDSSDSSDSSNSSD | |
| | SSDSSDSSDSSDSSNSSDSNDSSNSSDSSDSSNSSDSSNSSDSSDSSDSSDSD | |
| | SSNSSDSSNSSDSSDSSNSSDSSDSSDSSDGSDSDSSNRSDSSNSSDSSDSSD | |
| | SSNSSDSSDSSDSNESSNSSDSSDSSNSSDSDSSDSSNSSDSSDSSNSSDSSE | |
| | SSNSSDNSNSSDSSNSSDSSDSSDSSNSSDSSNSSDSSNSSDSSDSNSSDSSD | |
| | SSNSSDSSDSSDSSDSSDSSDSSNSSDSSDSSDSSDSSNSSDSSNSSDSSNSS | |
| | DSSDSSDSSDSSDSSDSSDSSDSSNSSDSSDSSDSSDSSDSSDSSDSSDSSES | |
| | SDSSDSSNSSDSSDSSDSSDSSDSSDSSDSSDSSDSSNSSDSSDSSDSSDSSD | |
| | SSNSSDSSDSSESSDSSDSSDSSDSSDSSDSSDSSDSSDSSNSSDSSDSSDSS | |
| | DSSDSSDSSDSSDSSDSSDSSDSSDSSDSSDSSDSSDSSDSNESSDSSDSSDS | |
| | SDSSNSSDSSDSSDSSDSTSDSNDESDSQSKSGNGNNNGSDSDSDSEGSDS | |
| | NHSTSDD | |
| DPP isoform | DDPNSSDESNGNDDANSESDNNSSSRGDASYNSDESKDNGNGSDSKGAE | 468 |
| 3 | DDDSDSTSDTNNSDSNGNGNNGNDDNDKSDSGKGKSDSSDSDSSDSSNS | |
| | SDSSDSSDSDSSDSNSSSDSDSSDSDSSDSSDSDSSDSSNSSDSSDSSDSSDS | |
| | SDSSDSSDSKSDSSKSESDSSDSDSKSDSSDSNSSDSSDNSDSSDSSNSSNS | |
| | SDSSDSSDSSDSSSSSDSSNSSDSSDSSDSSNSSESSDSSDSSDSDSSDSSDSS | |
| | NSNSSDSDSSNSSDSSDSSNSSDSSDSSDSSNSSDSSDSSDSSNSSDSSDSSD | |
| | SSDSSDSSNSSDSNDSSNSSDSSDSSNSSDSSNSSDSSDSSDSSDSDSSNSSD | |
| | SSNSSDSSDSSNSSDSSDSSDSSDGSDSDSSNRSDSSNSSDSSDSSDSSNSSD | |
| | SSDSSDSNESSNSSDSSDSSNSSDSDSSDSSNSSDSSDSSNSSDSSESSNSSD | |
| | NSNSSDSSNSSDSSDSSDSSNSSDSSNSSDSSNSSDSSDSNSSDSSDSSNSSD | |
| | SSDSSDSSDSSDSSDSSNSSDSSDSSDSSDSSNSSDSSNSSDSSNSSDSSDSS | |
| | DSSDSSDSSDSSDSSDSSNSSDSSDSSDSSDSSDSSDSSDSSDSSESSDSSDS | |
| | SNSSDSSDSSDSSDSSDSSDSSDSSDSSDSSNSSDSSDSSDSSDSSDSSNSSD | |
| | SSDSSESSDSSDSSDSSDSSDSSDSSDSSDSSDSSNSSDSSDSSDSSDSSDSS | |
| | DSSDSSDSSDSSDSSDSSDSSDSSDSSDSSDSSDSNESSDSSDSSDSSDSSNS | |
| | SDSSDSSDSSDSTSDSNDESDSQSKSGNGNNNGSDSDSDSEGSDSNHSTSD | |
| | D | |
| GDF-1 | DAEPVLGGGPGGACRARRLYVSFREVGWHRWVIAPRGFLANYCQGQCA | 469 |
| | LPVALSGSGGPPALNHAVLRALMHAAAPGAADLPCCVPARLSPISVLFFD | |
| | NSDNVVLRQYEDMVVDECGCR | |
| GDF-3 | AAIPVPKLSCKNLCHRHQLFINFRDLGWHKWIIAPKGFMANYCHGECPFS | 470 |
| | LTISLNSSNYAFMQALMHAVDPEIPQAVCIPTKLSPISMLYQDNNDNVILR | |
| | HYEDMVVDECGCG | |
| GDF-5 | APLATRQGKRPSKNLKARCSRKALHVNFKDMGWDDWIIAPLEYEAFHC | 471 |
| | EGLCEFPLRSHLEPTNHAVIQTLMNSMDPESTPPTCCVPTRLSPISILFIDSA | |
| | NNVVYKQYEDMVVESCGCR | |
| GDF8 | DFGLDCDEHSTESRCCRYPLTVDFEAFGWDWIIAPKRYKANYCSGECEF | 472 |
| | VFLQKYPHTHLVHQANPRGSAGPCCTPTKMSPINMLYFNGKEQIIYGKIP | |
| | AMVVDRCGCS | |
| GDF15 | ARARNGDHCPLGPGRCCRLHTVRASLEDLGWADWVLSPREVQVTMCIG | 473 |
| | ACPSQFRAANMHAQIKTSLHRLKPDTVPAPCCVPASYNPMVLIQKTDTG | |
| | VSLQTYDDLLAKDCHCI | |
| VEGF | APMAEGGGQNHHEVVKFMDVYQRSYCHPIETLVDIFQEYPDEIEYIFKPS | 474 |
| | CVPLMRCGGCCNDEGLECVPTEESNITMQIMRIKPHQGQHIGEMSFLQHN | |
| | KCECRPKKDRARQEKKSVRGKGKGQKRKRKKSRYKSWSVYVGARCCL | |
| | MPWSLPGPHPCGPCSERRKHLFVQDPQTCKCSCKNTDSRCKARQLELNE | |
| | RTCRCDKPRR | |
| HABP | FSLMSLLESLDPDWTPDQYDYSYEDYNQEENTSSTLTHAENPDWYYTED | 475 |
| Isoform 1 | QADPCQPNPCEHGGDCLVHGSTFTCSCLAPFSGNKCQKVQNTCKDNPCG | |
| | RGQCLITQSPPYYRCVCKHPYTGPSCSQVVPVCRPNPCQNGATCSRHKRR | |
| | SKFTCACPDQFKGKFCEIGSDDCYVGDGYSYRGKMNRTVNQHACLYWN | |
| | SHLLLQENYNMFMEDAETHGIGEHNFCRNPDADEKPWCFIKVTNDKVK | |
| | WEYCDVSACSAQDVAYPEESPTEPSTKLPGFDSCGKTEIAERKIKR | |
| HABP | IYGGFKSTAGKHPWQASLQSSLPLTISMPQGHFCGGALIHPCWVLTAAHC | 477 |
| Isoform 2 | TDIKTRHLKVVLGDQDLKKEEFHEQSFRVEKIFKYSHYNERDEIPHNDIAL | |
| | LKLKPVDGHCALESKYVKTVCLPDGSFPSGSECHISGWGVTETGKGSRQ | |
| | LLDAKVKLIANTLCNSRQLYDHMIDDSMICAGNLQKPGQDTCQGDSGGP | |
| | LTCEKDGTYYVYGIVSWGLECGKRPGVYTQVTKFLNWIKATIKSESGF | |
| CBP | AEVKKPAAAAAPGTAEKLSPKAATLAERSAGLAFSLYQAMAKDQAVEN | 479 |
| | ILVSPVVVASSLGLVSLGGKATTASQAKAVLSAEQLRDEEVHAGLGELLR | |
| | SLSNSTARNVTWKLGSRLYGPSSVSFADDFVRSSKQHYNCEHSKINFRDK | |
| | RSALQSINEWAAQTTDGKLPEVTKDVERTDGALLVNAMFFKPHWDEKF | |
| | HHKMVDNRGFMVTRSYTVGVMMMHRTGLYNYYDDEKEKLQIVEMPL | |
| | AHKLSSLIILMPHHVEPLERLEKLLTKEQLKIWMGKMQKKAVAISLPKGV | |
| | VEVTHDLQKHLAGLGLTEAIDKNKADLSRMSGKKDLYLASVFHATAFEL | |
| | DTDGNPFDQDIYGREELRSPKLFYADHPFIFLVRDTQSGSLLFIGRLVRPK | |
| | GDKMRDEL | |
| KGF | CNDMTPEQMATNVNCSSPERHTRSYDYMEGGDIRVRRLFCRTQWYLRID | 480 |
| | KRGKVKGTQEMKNNYNIMEIRTVAVGIVAIKGVESEFYLAMNKEGKLY | |
| | AKKECNEDCNFKELILENHYNTYASAKWTHNGGEMFVALNQKGIPVRG | |
| | KKTKKEQKTAHFLPMAIT | |
| TNFα | GPQREEFPRDLSLISPLAQAVRSSSRTPSDKPVAHVVANPQAEGQLQWLN | 481 |
| | RRANALLANGVELRDNQLVVPSEGLYLIYSQVLFKGQGCPSTHVLLTHTI | |
| | SRIAVSYQTKVNLLSAIKSPCQRETPEGAEAKPWYEPIYLGGVFQLEKGD | |
| | RLSAEINRPDYLDFAESGQVYFGIIAL | |
| TRAIL | TNELKQMQDKYSKSGIACFLKEDDSYWDPNDEESMNSPCWQVKWQLR | 482 |
| | QLVRKMILRTSEETISTVQEKQQNISPLVRERGPQRVAAHITGTRGRSNTL | |
| | SSPNSKNEKALGRKINSWESSRSGHSFLSNLHLRNGELVIHEKGFYYIYSQ | |
| | TYFRFQEEIKENTKNDKQMVQYIYKYTSYPDPILLMKSARNSCWSKDAE | |
| | YGLYSIYQGGIFELKENDRIFVSVTNEHLIDMDHEASFFGAFLVG | |
| WNT1 | ANSSGRWWGIVNVASSTNLLTDSKSLQLVLEPSLQLLSRKQRRLIRQNPG | 483 |
| | ILHSVSGGLQSAVRECKWQFRNRRWNCPTAPGPHLFGKIVNRGCRETAFI | |
| | FAITSAGVTHSVARSCSEGSIESCTCDYRRRGPGGPDWHWGGCSDNIDFG | |
| | RLFGREFVDSGEKGRDLRFLMNLHNNEAGRTTVFSEMRQECKCHGMSG | |
| | SCTVRTCWMRLPTLRAVGDVLRDRFDGASRVLYGNRGSNRASRAELLR | |
| | LEPEDPAHKPPSPHDLVYFEKSPNFCTYSGRLGTAGTAGRACNSSSPALD | |
| | GCELLCCGRGHRTRTQRVTERCNCTFHWCCHVSCRNCTHTRVLHECL | |
| WNT2 | SWWYMRATGGSSRVMCDNVPGLVSSQRQLCHRHPDVMRAISQGVAEW | 484 |
| | TAECQHQFRQHRWNCNTLDRDHSLFGRVLLRSSRESAFVYAISSAGVVF | |
| | AITRACSQGEVKSCSCDPKKMGSAKDSKGIFDWGGCSDNIDYGIKFARAF | |
| | VDAKERKGKDARALMNLHNNRAGRKAVKRFLKQECKCHGVSGSCTLR | |
| | TCWLAMADFRKTGDYLWRKYNGAIQVVMNQDGTGFTVANERFKKPTK | |
| | NDLVYFENSPDYCIRDREAGSLGTAGRVCNLTSRGMDSCEVMCCGRGY | |
| | DTSHVTRMTKCGCKFHWCCAVRCQDCLEALDVHTCKAPKNADWTTAT | |
| WNT2B | SWWYIGALGARVICDNIPGLVSRQRQLCQRYPDIMRSVGEGAREWIREC | 485 |
| | QHQFRHHRWNCTTLDRDHTVFGRVMLRSSREAAFVYAISSAGVVHAITR | |
| | ACSQGELSVCSCDPYTRGRHHDQRGDFDWGGCSDNIHYGVRFAKAFVD | |
| | AKEKRLKDARALMNLHNNRCGRTAVRRFLKLECKCHGVSGSCTLRTCW | |
| | RALSDFRRTGDYLRRRYDGAVQVMATQDGANFTAARQGYRRATRTDL | |
| | VYFDNSPDYCVLDKAAGSLGTAGRVCSKTSKGTDGCEIMCCGRGYDTT | |
| | RVTRVTQCECKFHWCCAVRCKECRNTVDVHTCKAPKKAEWLDQT | |
| WNT3 | GYPIWWSLALGQQYTSLGSQPLLCGSIPGLVPKQLRFCRNYIEIMPSVAEG | 486 |
| | VKLGIQECQHQFRGRRWNCTTIDDSLAIFGPVLDKATRESAFVHAIASAG | |
| | VAFAVTRSCAEGTSTICGCDSHHKGPPGEGWKWGGCSEDADFGVLVSRE | |
| | FADARENRPDARSAMNKHNNEAGRTTILDHMHLKCKCHGLSGSCEVKT | |
| | CWWAQPDFRAIGDFLKDKYDSASEMVVEKHRESRGWVETLRAKYSLFK | |
| | PPIERDLVYYENSPNFCEPNPETGSFGTRDRTCNVTSHGIDGCDLLCCGR | |
| | GHNTRTEKRKEKCHCIFHWCCYVSCQECIRIYDVHTCK | |
| WNT3A | SYPIWWSLAVGPQYSSLGSQPILCASIPGLVPKQLRFCRNYVEIMPSVAEG | 487 |
| | IKIGIQECQHQFRGRRWNCTTVHDSLAIFGPVLDKATRESAFVHAIASAG | |
| | VAFAVTRSCAEGTAAICGCSSRHQGSPGKGWKWGGCSEDIEFGGMVSRE | |
| | FADARENRPDARSAMNRHNNEAGRQAIASHMHLKCKCHGLSGSCEVKT | |
| | CWWSQPDFRAIGDFLKDKYDSASEMVVEKHRESRGWVETLRPRYTYFK | |
| | VPTERDLVYYEASPNFCEPNPETGSFGTRDRTCNVSSHGIDGCDLLCCGR | |
| | GHNARAERRREKCRCVFHWCCYVSCQECTRVYDVHTCK | |
| WNT4 | SNWLYLAKLSSVGSISEEETCEKLKGLIQRQVQMCKRNLEVMDSVRRGA | 488 |
| | QLAIEECQYQFRNRRWNCSTLDSLPVFGKVVTQGTREAAFVYAISSAGV | |
| | AFAVTRACSSGELEKCGCDRTVHGVSPQGFQWSGCSDNIAYGVAFSQSF | |
| | VDVRERSKGASSSRALMNLHNNEAGRKAILTHMRVECKCHGVSGSCEV | |
| | KTCWRAVPPFRQVGHALKEKFDGATEVEPRRVGSSRALVPRNAQFKPHT | |
| | DEDLVYLEPSPDFCEQDMRSGVLGTRGRTCNKTSKAIDGCELLCCGRGF | |
| | HTAQVELAERCSCKFHWCCFVKCRQCQRLVELHTCR | |
| WNT5A | IIGAQPLCSQLAGLSQGQKKLCHLYQDHMQYIGEGAKTGIKECQYQFRH | 489 |
| | RRWNCSTVDNTSVFGRVMQIGSRETAFTYAVSAAGVVNAMSRACREGE | |
| | LSTCGCSRAARPKDLPRDWLWGGCGDNIDYGYRFAKEFVDARERERIHA | |
| | KGSYESARILMNLHNNEAGRRTVYNLADVACKCHGVSGSCSLKTCWLQ | |
| | LADFRKVGDALKEKYDSAAAMRLNSRGKLVQVNSRFNSPTTQDLVYIDP | |
| | SPDYCVRNESTGSLGTQGRLCNKTSEGMDGCELMCCGRGYDQFKTVQT | |
| | ERCHCKFHWCCYVKCKKCTEIVDQFVCK | |
| WNT5B | QLLTDANSWWSLALNPVQRPEMFIIGAQPVCSQLPGLSPGQRKLCQLYQ | 490 |
| | EHMAYIGEGAKTGIKECQHQFRQRRWNCSTADNASVFGRVMQIGSRETA | |
| | FTHAVSAAGVVNAISRACREGELSTCGCSRTARPKDLPRDWLWGGCGD | |
| | NVEYGYRFAKEFVDAREREKNFAKGSEEQGRVLMNLQNNEAGRRAVY | |
| | KMADVACKCHGVSGSCSLKTCWLQLAEFRKVGDRLKEKYDSAAAMRV | |
| | TRKGRLELVNSRFTQPTPEDLVYVDPSPDYCLRNESTGSLGTQGRLCNKT | |
| | SEGMDGCELMCCGRGYNQFKSVQVERCHCKFHWCCFVRCKKCTEIVDQ | |
| | YICK | |
| WNT6 | LWWAVGSPLVMDPTSICRKARRLAGRQAELCQAEPEVVAELARGARLG | 491 |
| | VRECQFQFRFRRWNCSSHSKAFGRILQQDIRETAFVFAITAAGASHAVTQ | |
| | ACSMGELLQCGCQAPRGRAPPRPSGLPGTPGPPGPAGSPEGSAAWEWGG | |
| | CGDDVDFGDEKSRLFMDARHKRGRGDIRALVQLHNNEAGRLAVRSHTR | |
| | TECKCHGLSGSCALRTCWQKLPPFREVGARLLERFHGASRVMGTNDGK | |
| | ALLPAVRTLKPPGRADLLYAADSPDFCAPNRRTGSPGTRGRACNSSAPDL | |
| | SGCDLLCCGRGHRQESVQLEENCLCRFHWCCVVQCHRCRVRKELSLCL | |
| WNT7A | LGASIICNKIPGLAPRQRAICQSRPDAIIVIGEGSQMGLDECQFQFRNGRW | 492 |
| | NCSALGERTVFGKELKVGSREAAFTYAIIAAGVAHAITAACTQGNLSDCG | |
| | CDKEKQGQYHRDEGWKWGGCSADIRYGIGFAKVFVDAREIKQNARTLM | |
| | NLHNNEAGRKILEENMKLECKCHGVSGSCTTKTCWTTLPQFRELGYVLK | |
| | DKYNEAVHVEPVRASRNKRPTFLKIKKPLSYRKPMDTDLVYIEKSPNYCE | |
| | EDPVTGSVGTQGRACNKTAPQASGCDLMCCGRGYNTHQYARVWQCNC | |
| | KFHWCCYVKCNTCSER1EMYTCK | |
| WNT7B | ALSSVVALGANIICNKIPGLAPRQRAICQSRPDAIIVIGEGAQMGINECQYQ | 493 |
| | FRFGRWNCSALGEKTVFGQELRVGSREAAFTYAITAAGVAHAVTAACSQ | |
| | GNLSNCGCDREKQGYYNQAEGWKWGGCSADVRYGIDFSRRFVDAREIK | |
| | KNARRLMNLHNNEAGRKVLEDRMQLECKCHGVSGSCTTKTCWTTLPKF | |
| | REVGHLLKEKYNAAVQVEVVRASRLRQPTFLRIKQLRSYQKPMETDLVY | |
| | IEKSPNYCEEDAATGSVGTQGRLCNRTSPGADGCDTMCCGRGYNTHQYT | |
| | KVWQCNCKFHWCCFVKCNTCSERTEVFTCK | |
| WNT8A | VNNFLITGPKAYLTYTTSVALGAQSGIEECKFQFAWERWNCPENALQLST | 494 |
| | HNRLRSATRETSFIHAISSAGVMYIITKNCSMGDFENCGCDGSNNGKTGG | |
| | HGWIWGGCSDNVEFGERISKLFVDSLEKGKDARALMNLHNNRAGRLAV | |
| | RATMKRTCKCHGISGSCSIQTCWLQLAEFREMGDYLKAKYDQALKIEMD | |
| | KRQLRAGNSAEGHWVPAEAFLPSAEAELIFLEESPDYCTCNSSLGIYGTE | |
| | GRECLQNSHNTSRWERRSCGRLCTECGLQVEERKTEVISSCNCKFQWCC | |
| | TVKCDQCRHVVSKYYCARSPGSAQSLGKGSA | |
| WNT8B | WSVNNFLMTGPKAYLIYSSSVAAGAQSGIEECKYQFAWDRWNCPERAL | 495 |
| | QLSSHGGLRSANRETAFVHAISSAGVMYTLTRNCSLGDFDNCGCDDSRN | |
| | GQLGGQGWLWGGCSDNVGFGEAISKQFVDALETGQDARAAMNLHNNE | |
| | AGRKAVKGTMKRTCKCHGVSGSCTTQTCWLQLPEFREVGAHLKEKYHA | |
| | ALKVDLLQGAGNSAAGRGAIADTFRSISTRELVHLEDSPDYCLENKTLGL | |
| | LGTEGRECLRRGRALGRWERRSCRRLCGDCGLAVEERRAETVSSCNCKF | |
| | HWCCAVRCEQCRRRVTKYFCSRAERPRGGAAHKPGRKP | |
| WNT9A | YFGLTGSEPLTILPLTLEPEAAAQAHYKACDRLKLERKQRRMCRRDPGV | 496 |
| | AETLVEAVSMSALECQFQFRFERWNCTLEGRYRASLLKRGFKETAFLYAI | |
| | SSAGLTHALAKACSAGRMERCTCDEAPDLENREAWQWGGCGDNLKYSS | |
| | KFVKEFLGRRS SKDLRARVDFHNNLVGVKVIKAGVETTCKCHGVSGSCT | |
| | VRTCWRQLAPFHEVGKHLKHKYETALKVGSTTNEAAGEAGAISPPRGRA | |
| | SGAGGSDPLPRTPELVHLDDSPSFCLAGRFSPGTAGRRCHREKNCESICCG | |
| | RGHNTQSRVVTRPCQCQVRWCCYVECRQCTQREEVYTCKG | |
| WNT9B | SYFGLTGREVLTPFPGLGTAAAPAQGGAHLKQCDLLKLSRRQKQLCRRE | 497 |
| | PGLAETLRDAAHLGLLECQFQFRHERWNCSLEGRMGLLKRGFKETAFLY | |
| | AVSSAALTHTLARACSAGRMERCTCDDSPGLESRQAWQWGVCGDNLK | |
| | YSTKFLSNFLGSKRGNKDLRARADAHNTHVGIKAVKSGLRTTCKCHGVS | |
| | GSCAVRTCWKQLSPFRETGQVLKLRYDSAVKVSSATNEALGRLELWAP | |
| | ARQGSLTKGLAPRSGDLVYMEDSPSFCRPSKYSPGTAGRVCSREASCSSL | |
| | CCGRGYDTQSRLVAFSCHCQVQWCCYVECQQCVQEELVYTCKH | |
| WNT10A | MPRSAPNDILDLRLPPEPVLNANTVCLTLPGLSRRQMEVCVRHPDVAASA | 498 |
| | IQGIQIAIHECQHQFRDQRWNCSSLETRNKIPYESPIFSRGFRESAFAYAIA | |
| | AAGVVHAVSNACALGKLKACGCDASRRGDEEAFRRKLHRLQLDALQRG | |
| | KGLSHGVPEHPALPTASPGLQDSWEWGGCSPDMGFGERFSKDFLDSREP | |
| | HRDIHARMRLHNNRVGRQAVMENMRRKCKCHGTSGSCQLKTCWQVTP | |
| | EFRTVGALLRSRFHRATLIRPHNRNGGQLEPGPAGAPSPAPGAPGPRRRA | |
| | SPADLVYFEKSPDFCEREPRLDSAGTVGRLCNKSSAGSDGCGSMCCGRG | |
| | HNILRQTRSERCHCRFHWCCFVVCEECRITEWVSVCK | |
| WNT10B | NEILGLKLPGEPPLTANTVCLTLSGLSKRQLGLCLRNPDVTASALQGLHIA | 499 |
| | VHECQHQLRDQRWNCSALEGGGRLPHHSAILKRGFRESAFSFSMLAAGV | |
| | MHAVATACSLGKLVSCGCGWKGSGEQDRLRAKLLQLQALSRGKSFPHS | |
| | LPSPGPGSSPSPGPQDTWEWGGCNHDMDFGEKFSRDFLDSREAPRDIQAR | |
| | MRIHNNRVGRQVVTENLKRKCKCHGTSGSCQFKTCWRAAPEFRAVGAA | |
| | LRERLGRAIFIDTHNRNSGAFQPRLRPRRLSGELVYFEKSPDFCERDPTMG | |
| | SPGTRGRACNKTSRLLDGCGSLCCGRGHNVLRQTRVERCHCRFHWCCY | |
| | VLCDECKVTEWVNVCK | |
| WNT11 | IKWLALSKTPSALALNQTQHCKQLEGLVSAQVQLCRSNLELMHTVVHA | 500 |
| | AREVMKACRRAFADMRWNCSSIELAPNYLLDLERGTRESAFVYALSAA | |
| | AISHAIARACTSGDLPGCSCGPVPGEPPGPGNRWGGCADNLSYGLLMGA | |
| | KFSDAPMKVKKTGSQANKLMRLHNSEVGRQALRASLEMKCKCHGVSGS | |
| | CSIRTCWKGLQELQDVAADLKTRYLSATKVVHRPMGTRKHLVPKDLDIR | |
| | PVKDSELVYLQSSPDFCMKNEKVGSHGTQDRQCNKTSNGSDSCDLMCC | |
| | GRGYNPYTDRVVERCHCKYHWCCYVTCRRCERTVERYVCK | |
| WNT16 | NWMWLGIASFGVPEKLGCANLPLNSRQKELCKRKPYLLPSIREGARLGIQ | 501 |
| | ECGSQFRHERWNCMITAAATTAPMGASPLFGYELSSGTKETAFIYAVMA | |
| | AGLVHSVTRSCSAGNMTECSCDTTLQNGGSASEGWHWGGCSDDVQYG | |
| | MWFSRKFLDFPIGNTTGKENKVLLAMNLHNNEAGRQAVAKLMSVDCRC | |
| | HGVSGSCAVKTCWKTMSSFEKIGHLLKDKYENSIQISDKTKRKMRRREK | |
| | DQRKIPIHKDDLLYVNKSPNYCVEDKKLGIPGTQGRECNRTSEGADGCNL | |
| | LCCGRGYNTHVVRHVERCECKFIWCCYVRCRRCESMTDVHTCK | |

Targeting Moieties

In some embodiments, the device or kit comprises a targeting moiety that tethers the therapeutic agent to the structure. In some embodiments, the targeting moiety is connected to the therapeutic agent, and the moiety non-covalently binds to the structure. As a non-limiting example, the targeting moiety is covalently connected to the therapeutic agent via a peptide bond. For instance, targeting moiety comprises a targeting peptide, and the targeting peptide is linked to the therapeutic agent via a peptide bond.

In some embodiments, the targeting moiety has an affinity for the structure, or a component of the structure, e.g., to a ceramic material of the structure such as calcium phosphate. In some embodiments, the dissociation constant (KD) for binding between the targeting moiety and the structure or component thereof is: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM. For example, the targeting moiety may bind to beta-tricalcium phosphate with an affinity of about 100 fM to about 100 μM, about 1 pM to about 100 μM, about 10 pM to about 100 μM, about 100 pM to about 100 μM, or about 1 μM to about 100 μM.

In some embodiments, the targeting moiety comprises one or more targeting peptides that each bind to the structure. In some embodiments, the targeting peptide binds to the ceramic material of the structure. For example, the targeting peptide binds to calcium phosphate (e.g., tricalcium phosphate, beta tricalcium phosphate, alpha tricalcium phosphate), hydroxyapatite, fluorapatite, bone (e.g., demineralized bone), glasses (bioglasses) such as silicates, vanadates, and related ceramic minerals, or chelated divalent metal ions, or a combination thereof. In some embodiments, the targeting peptide comprises two or more targeting peptides. In some embodiments, two or more targeting peptides is no more than about 50, 45, 40, 35, 30, 25, 20, 15, or 10 targeting peptides. In some embodiments, two or more targeting peptides is about 2, 3, 4, 5, 6, 7, 8, 9, 10, 11, 12, 13, 14, 15, 20, 25, or 30 targeting peptides. In some embodiments, two or more targeting peptides is about 2 to about 10 targeting peptides. In some embodiments, two or more targeting peptides is about 5 targeting peptides.

In some embodiments, the targeting peptide comprises a sequence at least about 70%, 75%, 80%, 85%, 90%, 95%, or 100% identical to SEQ ID NO: 1. In some embodiments, the targeting peptide comprises a sequence at least about 70%, 75%, 80%, 85%, 90%, 95%, or 100% identical to SEQ ID NO: 2. In some embodiments, the targeting peptide comprises a sequence at least about 70%, 75%, 80%, 85%, 90%, 95%, or 100% identical to SEQ ID NO: 3. In some embodiments, the targeting peptide comprises a sequence at least about 70%, 75%, 80%, 85%, 90%, 95%, or 100% identical to SEQ ID NO: 4. In some embodiments, the targeting peptide comprises a sequence at least about 70%, 75%, 80%, 85%, 90%, 95%, or 100% identical to SEQ ID NO: 5. In some embodiments, the targeting peptide comprises a sequence at least about 70%, 75%, 80%, 85%, 90%, 95%, or 100% identical to SEQ ID NO: 6. In some embodiments, the targeting peptide comprises a sequence at least about 70%, 75%, 80%, 85%, 90%, 95%, or 100% identical to SEQ ID NO: 7. In some embodiments, the targeting peptide comprises a sequence at least about 70%, 75%, 80%, 85%, 90%, 95%, or 100% identical to SEQ ID NO: 8. In some embodiments, the targeting peptide comprises a sequence at least about 70%, 75%, 80%, 85%, 90%, 95%, or 100% identical to SEQ ID NO: 9. In some embodiments, the targeting peptide comprises a sequence at least about 70%, 75%, 80%, 85%, 90%, 95%, or 100% identical to SEQ ID NO: 10. In some embodiments, the targeting peptide comprises a sequence at least about 70%, 75%, 80%, 85%, 90%, 95%, or 100% identical to SEQ ID NO: 11. In some embodiments, the targeting peptide comprises a sequence at least about 70%, 75%, 80%, 85%, 90%, 95%, or 100% identical to SEQ ID NO: 12. In some embodiments, the targeting peptide comprises a sequence at least about 70%, 75%, 80%, 85%, 90%, 95%, or 100% identical to SEQ ID NO: 13. In some embodiments, the targeting peptide comprises a sequence at least about 70%, 75%, 80%, 85%, 90%, 95%, or 100% identical to SEQ ID NO: 14. In some embodiments, the targeting peptide comprises a sequence at least about 70%, 75%, 80%, 85%, 90%, 95%, or 100% identical to SEQ ID NO: 15. In some embodiments, the targeting peptide comprises a sequence at least about 70%, 75%, 80%, 85%, 90%, 95%, or 100% identical to SEQ ID NO: 16. In some embodiments, the targeting peptide comprises a sequence at least about 70%, 75%, 80%, 85%, 90%, 95%, or 100% identical to SEQ ID NO: 17. In some embodiments, the targeting peptide comprises a sequence at least about 70%, 75%, 80%, 85%, 90%, 95%, or 100% identical to SEQ ID NO: 18. In some embodiments, the targeting peptide comprises a sequence at least about 70%, 75%, 80%, 85%, 90%, 95%, or 100% identical to SEQ ID NO: 19. In some embodiments, the targeting peptide comprises a sequence at least about 70%, 75%, 80%, 85%, 90%, 95%, or 100% identical to SEQ ID NO: 20. In some embodiments, the targeting peptide comprises a sequence at least about 70%, 75%, 80%, 85%, 90%, 95%, or 100% identical to SEQ ID NO: 21. In some embodiments, the targeting peptide comprises a sequence at least about 70%, 75%, 80%, 85%, 90%, 95%, or 100% identical to SEQ ID NO: 22. In some embodiments, the targeting peptide comprises a sequence at least about 70%, 75%, 80%, 85%, 90%, 95%, or 100% identical to SEQ ID NO: 23. In some embodiments, the targeting peptide comprises a sequence at least about 70%, 75%, 80%, 85%, 90%, 95%, or 100% identical to SEQ ID NO: 24. In some embodiments, the targeting peptide comprises a sequence at least about 70%, 75%, 80%, 85%, 90%, 95%, or 100% identical to SEQ ID NO: 25. In some embodiments, the targeting peptide comprises a sequence at least about 70%, 75%, 80%, 85%, 90%, 95%, or 100% identical to SEQ ID NO: 26. In some embodiments, the targeting peptide comprises a sequence at least about 70%, 75%, 80%, 85%, 90%, 95%, or 100% identical to SEQ ID NO: 27. In some embodiments, the targeting peptide comprises a sequence at least about 70%, 75%, 80%, 85%, 90%, 95%, or 100% identical to SEQ ID NO: 28. In some embodiments, the targeting peptide comprises a sequence at least about 70%, 75%, 80%, 85%, 90%, 95%, or 100% identical to SEQ ID NO: 29. In some embodiments, the targeting peptide comprises a sequence at least about 70%, 75%, 80%, 85%, 90%, 95%, or 100% identical to SEQ ID NO: 30. In some embodiments, the targeting peptide comprises a sequence at least about 70%, 75%, 80%, 85%, 90%, 95%, or 100% identical to SEQ ID NO: 31. In some embodiments, the targeting peptide comprises a sequence at least about 70%, 75%, 80%, 85%, 90%, 95%, or 100% identical to SEQ ID NO: 32. In some embodiments, the targeting peptide comprises a sequence at least about 70%, 75%, 80%, 85%, 90%, 95%, or 100% identical to SEQ ID NO: 33. In some embodiments, the targeting peptide comprises a sequence at least about 70%, 75%, 80%, 85%, 90%, 95%, or 100% identical to SEQ ID NO: 34. In some embodiments, the targeting peptide comprises a sequence at least about 70%, 75%, 80%, 85%, 90%, 95%, or 100% identical to SEQ ID NO: 35. In some embodiments, the targeting peptide comprises a sequence at least about 70%, 75%, 80%, 85%, 90%, 95%, or 100% identical to SEQ ID NO: 36. In some embodiments, the targeting peptide comprises a sequence at least about 70%, 75%, 80%, 85%, 90%, 95%, or 100% identical to SEQ ID NO: 37. In some embodiments, the targeting peptide comprises a sequence at least about 70%, 75%, 80%, 85%, 90%, 95%, or 100% identical to SEQ ID NO: 38. In some embodiments, the targeting peptide comprises a sequence at least about 70%, 75%, 80%, 85%, 90%, 95%, or 100% identical to SEQ ID NO: 39. In some embodiments, the targeting peptide comprises a sequence at least about 70%, 75%, 80%, 85%, 90%, 95%, or 100% identical to SEQ ID NO: 40. In some embodiments, the targeting peptide comprises a sequence at least about 70%, 75%, 80%, 85%, 90%, 95%, or 100% identical to SEQ ID NO: 41. In some embodiments, the targeting peptide comprises a sequence at least about 70%, 75%, 80%, 85%, 90%, 95%, or 100% identical to SEQ ID NO: 42. In some embodiments, the targeting peptide comprises a sequence at least about 70%, 75%, 80%, 85%, 90%, 95%, or 100% identical to SEQ ID NO: 43. In some embodiments, the targeting peptide comprises a sequence at least about 70%, 75%, 80%, 85%, 90%, 95%, or 100% identical to SEQ ID NO: 44. In some embodiments, the targeting peptide comprises a sequence at least about 70%, 75%, 80%, 85%, 90%, 95%, or 100% identical to SEQ ID NO: 45. In some embodiments, the targeting peptide comprises a sequence at least about 70%, 75%, 80%, 85%, 90%, 95%, or 100% identical to SEQ ID NO: 46. In some embodiments, the targeting peptide comprises a sequence at least about 70%, 75%, 80%, 85%, 90%, 95%, or 100% identical to SEQ ID NO: 47. In some embodiments, the targeting peptide comprises a sequence at least about 70%, 75%, 80%, 85%, 90%, 95%, or 100% identical to SEQ ID NO: 48. In some embodiments, the targeting peptide comprises a sequence at least about 70%, 75%, 80%, 85%, 90%, 95%, or 100% identical to SEQ ID NO: 49. In some embodiments, the targeting peptide comprises a sequence at least about 70%, 75%, 80%, 85%, 90%, 95%, or 100% identical to SEQ ID NO: 50. In some embodiments, the targeting peptide comprises a sequence at least about 70%, 75%, 80%, 85%, 90%, 95%, or 100% identical to SEQ ID NO: 51. In some embodiments, the targeting peptide comprises a sequence at least about 70%, 75%, 80%, 85%, 90%, 95%, or 100% identical to SEQ ID NO: 52. In some embodiments, the targeting peptide comprises a sequence at least about 70%, 75%, 80%, 85%, 90%, 95%, or 100% identical to SEQ ID NO: 53. In some embodiments, the targeting peptide comprises a sequence at least about 70%, 75%, 80%, 85%, 90%, 95%, or 100% identical to SEQ ID NO: 54. In some embodiments, the targeting peptide comprises a sequence at least about 70%, 75%, 80%, 85%, 90%, 95%, or 100% identical to SEQ ID NO: 55. In some embodiments, the targeting peptide comprises a sequence at least about 70%, 75%, 80%, 85%, 90%, 95%, or 100% identical to SEQ ID NO: 56. In some embodiments, the targeting peptide comprises a sequence at least about 70%, 75%, 80%, 85%, 90%, 95%, or 100% identical to SEQ ID NO: 57. In some embodiments, the targeting peptide comprises a sequence at least about 70%, 75%, 80%, 85%, 90%, 95%, or 100% identical to SEQ ID NO: 58. In some embodiments, the targeting peptide comprises a sequence at least about 70%, 75%, 80%, 85%, 90%, 95%, or 100% identical to SEQ ID NO: 59. In some embodiments, the targeting peptide comprises a sequence at least about 70%, 75%, 80%, 85%, 90%, 95%, or 100% identical to SEQ ID NO: 60. In some embodiments, the targeting peptide comprises a sequence at least about 70%, 75%, 80%, 85%, 90%, 95%, or 100% identical to SEQ ID NO: 61.

TABLE 5
Targeting Peptides

| | SEQ |
|---|---|
| | ID |
| Sequence | NO: |
| LLADTTHHRPWT | 1 |
| VIGESTHHRPWS | 2 |
| LIADSTHHSPWT | 3 |
| ILAESTHHKPWT | 4 |
| ILAETTHHRPWS | 5 |
| IIGESSHHKPFT | 6 |
| GLGDTTHHRPWG | 7 |
| VLGDTTHHKPWT | 8 |
| IVADSTHHRPWT | 9 |
| STADTSHHRPS | 10 |
| TSGGESTHHRPS | 11 |
| TSGGESSHHKPS | 12 |
| TGSGDSSHHRPS | 13 |
| GSSGESTHHKPST | 14 |
| VGADSTHHRPVT | 15 |
| GAADTTHHRPVT | 16 |
| AGADTTHHRPVT | 17 |
| GGADTTHHRPAT | 18 |
| GGADTTHHRPGT | 19 |
| LLADTTHHRPWTVIGESTHHRPWS | 20 |
| LLADTTHHRPWTVIGESTHHRPWSIIGESSHHKPFT | 21 |
| LLADTTHHRPWTVIGESTHHRPWSIIGESSHHKPFTGLGDTTHHRPWGILAESTHRKPWT | 22 |
| LLADTTHHRPWTILAESTHHKPWT | 23 |
| LLADTTHHRPWTILAESTHHKPWTLLADTTHHRPWTILAESTHHKPWTLLADTTHHRPW | 24 |
| T | |
| LLADTTHHRPWTGLGDTTHHRPWG | 25 |
| LLADTTHHRPWTGLGDTTHHRPWGLLADTTHHRPWT | 26 |
| LLADTTHHRPWTGLGDTTHHRPWGLLADTTHHRPWTGLGDTTHHRPWGLLADTTHHRP | 27 |
| WT | |
| LLADTTHHRPWTGLGDTTHHRPWGLLADTTHHRPWTGLGDTTHHRPWGLLADTTHHRP | 28 |
| WTGLGDTTHHRPWGLLADTTHHRPWT | |
| STADTSHHRPSTSGGESTHHRPSTSGGESSHHKPSTGSGDSSHHRPSGSSGESTHHKPST | 29 |
| VGADSTRHRPVTGAADTTHHRPVTAGADTTHHRPVTGGADTTHHRPATGGADTTHHRP | 30 |
| GT | |
| STADTSHHRPSLLADTTRHRPWTTSGGESTHHRPSVGADSTHHRPVTTSGGESSHHKPSG | 31 |
| AADTTHHRPVTTGSGDSSHHRPSGSSGESTHHKPSTGGADTTHHRPAT | |
| AAADTTHHRPWT | 32 |
| AAADTTRHRPWTAAADTTHHRPWTAAADTTHHRPWTAAADTTHHRPWTAAADTTHH | 33 |
| RPWT | |
| LLADAAHHRPWTLLADAAHHRPWTLLADAAHHRPWTLLADAAHHRPWTLLADAAHH | 34 |
| RPWT | |
| LLADTTAARPWTLLADTTAARPWTLLADTTAARPWTLLADTTAARPWTLLADTTAARP | 35 |
| WT | |
| LLADTTHHRPWTLLADTTHHRPWT | 36 |
| LLADTTHHRPWTLLADTTHHRPWTLLADTTHHRPWT | 37 |
| LLADTTHHRPWTLLADTTHHRPWTLLADTTHHRPWTLLADTTHHRPWTLLADTTHHRP | 38 |
| WT | |
| STSGSTVIGESTHHRPWSLIADSTHHSPWTILAESTHHKPWTILAETTHHRPWSIIGESSHH | 39 |
| KPFTGLGDTTHHRPWGVLGDTTHHKPWTIVADTHHRPWTGQVLPTTTPSSPSTTSGS | |
| LLADTTHHRPWTVIGESTHHRPWSIIGESSHHKPFTGLGDTTHHRPWG | 40 |
| VIGESTHHRPWSIIGESSHHKPFTGLGDTTHHRPWGILAESTHHKPWT | 41 |
| (X1)(X2), wherein X1 comprises SEQ ID NO: 1 and X2 comprises one or more of SEQ ID NOS: | 42 |
| 1-41. | |
| (X1)(X2), wherein X1 comprises SEQ ID NO: 2 and X2 comprises one or more of SEQ ID NOS: | 43 |
| 1-41. | |
| (X1)(X2), wherein X1 comprises SEQ ID NO: 4 and X2 comprises one or more of SEQ ID NOS: | 44 |
| 1-41. | |
| (X1)(X2), wherein X1 comprises SEQ ID NO: 6 and X2 comprises one or more of SEQ ID NOS: | 45 |
| 1-41. | |
| (X1)(X2), wherein X1 comprises SEQ ID NO: 7 and X2 comprises one or more of SEQ ID NOS: | 46 |
| 1-41. | |
| (X1)(X2), wherein X1 comprises SEQ ID NO: 1 and X2 comprises one or more of SEQ ID NOS: | 47 |
| 2, 4, 6, or 7. | |
| (X1)(X2), wherein X1 comprises SEQ ID NO: 2 and X2 comprises one or more of SEQ ID NOS: | 48 |
| 1, 4, 6, or 7. | |
| (X1)(X2), wherein X1 comprises SEQ ID NO: 4 and X2 comprises one or more of SEQ ID NOS: | 49 |
| 1, 2, 6, or 7. | |
| (X1)(X2), wherein X1 comprises SEQ ID NO: 6 and X2 comprises one or more of SEQ ID NOS: | 50 |
| 1, 4, 2, or 7. | |
| (X1)(X2), wherein X1 comprises SEQ ID NO: 7 and X2 comprises one or more of SEQ ID NOS: | 51 |
| 1, 4, 6, or 2. | |
| (X1)(X2), wherein X1 comprises SEQ ID NO: 1 and X2 comprises two or more of SEQ ID NOS: | 52 |
| 2, 4, 6, or 7. | |
| (X1)(X2), wherein X1 comprises SEQ ID NO: 2 and X2 comprises two or more of SEQ ID NOS: | 53 |
| 1, 4, 6, or 7. | |
| (X1)(X2), wherein X1 comprises SEQ ID NO: 4 and X2 comprises two or more of SEQ ID NOS: | 54 |
| 1, 2, 6, or 7. | |
| (X1)(X2), wherein X1 comprises SEQ ID NO: 6 and X2 comprises two or more of SEQ ID NOS: | 55 |
| 1, 4, 2, or 7. | |
| (X1)(X2), wherein X1 comprises SEQ ID NO: 7 and X2 comprises two or more of SEQ ID NOS: | 56 |
| 1, 4, 6, or 2. | |
| (X1)(X2), wherein X1 comprises SEQ ID NO: 1 and X2 comprises three or more of SEQ ID NOS: | 57 |
| 2, 4, 6, or 7. | |
| (X1)(X2), wherein X1 comprises SEQ ID NO: 2 and X2 comprises three or more of SEQ ID NOS: | 58 |
| 1, 4, 6, or 7. | |
| (X1)(X2), wherein X1 comprises SEQ ID NO: 4 and X2 comprises three or more of SEQ ID NOS: | 59 |
| 1, 2, 6, or 7. | |
| (X1)(X2), wherein X1 comprises SEQ ID NO: 6 and X2 comprises three or more of SEQ ID NOS: | 60 |
| 1, 4, 2, or 7. | |
| (X1)(X2), wherein X1 comprises SEQ ID NO: 7 and X2 comprises three or more of SEQ ID NOS: | 61 |
| 1, 4, 6, or 2. | |

In some embodiments, a targeting peptide comprises one or more sequences of Table 5. In some embodiments, the targeting peptide comprises a sequence at least about 70%, 75%, 80%, 85%, 90%, 95%, or 100% identical to a sequence of Table 5.

TABLE 6
Additional Targeting Peptides

| | SEQ |
|---|---|
| | ID |
| Sequence | NO |
| ACAPLMFSQC | 62 |
| ACHASLKHRC | 63 |
| ACLSTKTNIC | 64 |
| ACTTPSKHQC | 65 |
| AHFSPNLLLGG | 66 |
| AHSLKSITNHGL | 67 |
| AKQTVPV | 68 |
| AKTLMPSPFPRT | 69 |
| AMSQTMTAAIEK | 70 |
| ANPPLSL | 71 |
| ANPYHRH | 72 |
| APLSLSL | 73 |
| APYHPTIPASVHGGGK | 74 |
| ASAVGSLSIRWX | 75 |
| ASGPTNV | 76 |
| ASHNPKL | 77 |
| ASWVDSRQPSAA | 78 |
| ATFSPPL | 79 |
| ATWSHHLSSAGL | 80 |
| ATWSHHLSSAGLGGGS | 81 |
| CAHLSPHKC | 82 |
| CDIPWRNEC | 83 |
| CDPLRQHSC | 84 |
| CDSLGHWLC | 85 |
| CDYTTRHSC | 86 |
| CHGTLNPEC | 87 |
| CHHNLSWEC | 88 |
| CHIWTLASC | 89 |
| CHNTFSPRC | 90 |
| CIPLHASLC | 91 |
| CITTTSLSC | 92 |
| CKLTTCKDC | 93 |
| CKNHTTFWC | 94 |
| CLKLLSRSC | 95 |
| CLLKAHPSC | 96 |
| CLNQLKQAC | 97 |
| CLSTKTNIC | 98 |
| CMNFPSPHC | 99 |
| CNYPTLKSC | 100 |
| CPQLTVGQHRT | 101 |
| CPQSPTYTC | 102 |
| CPSSAIHTC | 103 |
| CPTSTARIC | 104 |
| CQASSFPSC | 105 |
| CQPYFWYRC | 106 |
| CQTLTPSIC | 107 |
| CSKLGHLWC | 108 |
| CSKTPERIX | 109 |
| CSNNNRMTC | 110 |
| CSPILSLSC | 111 |
| CSPTNFTRC | 112 |
| CSRPAMNVC | 113 |
| CSTKAYPNC | 114 |
| CSTSSCGSC | 115 |
| CSYWGHRDC | 116 |
| CTAHDANAC | 117 |
| CTANSEKTC | 118 |
| CTHPKASMC | 119 |
| CTKTINGKC | 120 |
| CTNMQSPLC | 121 |
| CTPFTKLPC | 122 |
| CTPTTDSIC | 123 |
| CTQQNGHPC | 124 |
| CTTPSKHQC | 125 |
| CTYNVAKPC | 126 |
| DKLHRLA | 127 |
| DLNYFTLSSKRE | 128 |
| DLPPTLHTTGSP | 129 |
| DMRQQRS | 130 |
| DQYWGLR | 131 |
| DSSNPIFWRPSS | 132 |
| DSSNPIFWRPSSGGGS | 133 |
| EFLGVPASLVNP | 134 |
| EPNHTRF | 135 |
| EPRRAVAAL | 136 |
| EPRRAVAEL | 137 |
| EPRREVAEL | 138 |
| EPRREVCEL | 139 |
| ESDLTHALHWLG | 140 |
| ESLKSIS | 141 |
| ETRTQLL | 142 |
| ETVCASS | 143 |
| ETYARPL | 144 |
| ETYQQPL | 145 |
| EVHSTDRYRSIP | 146 |
| FGLQPTGDIARR | 147 |
| FSMDDPERVRSP | 148 |
| FSPLHTSTYRPS | 149 |
| FTLPTIR | 150 |
| FVNLLGQ | 151 |
| GDFNSGHHTTTR | 152 |
| GGGAAAA | 153 |
| GIHVPWMPPVAF | 154 |
| GIHVPWMPPVAFGGGS | 155 |
| GPSNNLPWSNTP | 156 |
| GSAGLKYPLYKS | 157 |
| GSCPPKK | 158 |
| GSLFKAL | 159 |
| GTQTPQP | 160 |
| GTSRLFS | 161 |
| GVHKHFYSRWLG | 162 |
| HAPLTRSPAPNL | 163 |
| HAPVQPN | 164 |
| HGSLTTLXRYEP | 165 |
| HHFHLPKLRPPV | 166 |
| HHQRSPA | 167 |
| HHTWDTRIWQAF | 168 |
| HMLAQTF | 169 |
| HNVTTRTQRLMP | 170 |
| HPTTPIHMPNF | 171 |
| HQFISPEPFLIS | 172 |
| HQFPXSNLVWKP | 173 |
| HQWDHKY | 174 |
| HRDPXSXPSAXRP | 175 |
| HRLGHMS | 176 |
| HSACHASLKHRC | 177 |
| HSACKLTTCKDG | 178 |
| HSACLSTKTNIC | 179 |
| HSMPHMGTYLLT | 180 |
| HSTGPTR | 181 |
| HTLLSTT | 182 |
| HYPTVNF | 183 |
| IAHVPETRLAQM | 184 |
| IFSMGTALARPL | 185 |
| IGYPVLP | 186 |
| INFQFLKPSTTR | 187 |
| INKHPQQVSTLL | 188 |
| IQHQAKT | 189 |
| IRXLXIS | 190 |
| ISPSHSQAQADL | 191 |
| KAFDKHG | 192 |
| KATITGM | 193 |
| KEIPPIPLLAPS | 194 |
| KEIPPIPLLAPSGGGS | 195 |
| KIPKACCVPTELSAISMLYL | 196 |
| KIPKASSVPTELSAIATLYL | 197 |
| AAAAEPRRAVAAL | |
| KIPKASSVPTELSAISTLYL | 198 |
| KIPKASSVPTELSAISTLYL | 199 |
| AAAAEPRRAVAAL | |
| KIPKASSVPTELSAISTLYL | 200 |
| AAAAEPRRAVAEL | |
| KIPKASSVPTELSAISTLYL | 201 |
| AAAAEPRREVAEL | |
| KIPKASSVPTELSAISTLYL | 202 |
| AAAAXPRRXVAXL | |
| KIPKASSVPTELSAISTLYL | 203 |
| XPRRXVAXL | |
| KLHASLA | 204 |
| KLSAWSF | 205 |
| KLTWQELYQLKYKGI | 206 |
| KLTWQELYQLKYKGIGGG | 207 |
| AAAAEPRREVAEL | |
| KMNHMPN | 208 |
| KPMQFVH | 209 |
| KTSSWAN | 210 |
| LASTTHV | 211 |
| LDYPIPQTVLHH | 212 |
| LFAAVPSTQFFR | 213 |
| LGFDPTSTRFYT | 214 |
| LGPGKAF | 215 |
| LKPFSGA | 216 |
| LLADTTHHRPWP | 217 |
| LLADTTHHRPWT | 218 |
| LLADTTHHRPWTGGGS | 219 |
| LPLKFK | 220 |
| LPFQPPI | 221 |
| LPLTPLP | 222 |
| LPRDLHATPQQI | 223 |
| LPSIHNL | 224 |
| LPWAPNLPDSTA | 225 |
| LPWTEPSFWRTP | 226 |
| LPWTEPSFWRTPGGGS | 227 |
| LQKSPSL | 228 |
| LQPSQPQRFAPT | 229 |
| LRAFPSLPHTVT | 230 |
| LSAPMEY | 231 |
| LSKNPLL | 232 |
| LSLRASAATDFQ | 233 |
| LSPPMQLQPTYS | 234 |
| LTPTMFNMHGVL | 235 |
| LTQTLQY | 236 |
| MHNVSDSNDSAI | 237 |
| MKVHERS | 238 |
| MPQTLVLPRSLL | 239 |
| MQFTPAPSPSDH | 240 |
| MTSQTLR | 241 |
| MYPLPAP | 242 |
| NERQMEL | 243 |
| NFAMNLR | 244 |
| NITQLGS | 245 |
| NKPLSTL | 246 |
| NNVSQKWQQRLI | 247 |
| NNVSQKWQQRLIGGGS | 248 |
| NPDHPDIPQDVHGGGK | 249 |
| NPMIMNQ | 250 |
| NPQMQRS | 251 |
| NPRSQAT | 252 |
| NPYAPTIPQSVAGGGK | 253 |
| NPYHPTIPQSVH | 254 |
| NPYHPTIPQSVHGGGK | 255 |
| NSMIAHNKTRMH | 256 |
| NSMIAHNKTRMHGGGS | 257 |
| NSSMLGMLPSSF | 258 |
| NTSSSQGTQRLG | 259 |
| NTTTDIPSPSQF | 260 |
| NYPTLKS | 261 |
| NYSHLRVKLPTP | 262 |
| NYSHLRVKLPTPGGGS | 263 |
| PAKQKAH | 264 |
| PDIPLSR | 265 |
| PGQWPSSLTLYK | 266 |
| PHNPGKL | 267 |
| PIDAFFD | 268 |
| PLTQPSH | 269 |
| PPKDSRG | 270 |
| PPNMARA | 271 |
| PSMKHWR | 272 |
| PTNKPHT | 273 |
| PTTMTRW | 274 |
| PTTWGHL | 275 |
| PXGPXGPXGPXGPXGPXA | 276 |
| PXGPXGPXGPXGPXGPXG | |
| QHNFRGASSSAP | 277 |
| QIPQMRILHPYG | 278 |
| QIQKPPRTPPSL | 279 |
| QLTQTMWKDTTL | 280 |
| QNLPPERYSEAT | 281 |
| QNPRQIY | 282 |
| QNYLLPK | 283 |
| QPGLWPS | 284 |
| QRSWTLDSALSM | 285 |
| QRSWTLDSALSMGGGS | 286 |
| QSLSFAGPPAWQ | 287 |
| QSSYNPI | 288 |
| QTHARHQ | 289 |
| QTHSSLW | 290 |
| QTTMTPLWPSFS | 291 |
| RCMSEVISFNCP | 292 |
| RHTLPLH | 293 |
| RPHTITN | 294 |
| RSPYYNKWSSKF | 295 |
| RTPLQPLEDFRP | 296 |
| SAGHIHEAHRPL | 297 |
| SAISDHRAHRSH | 298 |
| SAKGRAD | 299 |
| SAKKVFS | 300 |
| SASGTPS | 301 |
| SEPTYWRPNMSG | 302 |
| SFAPDIKYPVPS | 303 |
| SFQSMSLMTLVV | 304 |
| SFWHHHSPRSPL | 305 |
| SGHQLLLNKMPN | 306 |
| SGHQLLLNKMPNGGGS | 307 |
| SIFAHQTPTHKN | 308 |
| SIPKMIPTESLL | 309 |
| SIPSHSIHSAKA | 310 |
| SIRTSMNPPNLL | 311 |
| SKTSSTS | 312 |
| SLLTPWL | 313 |
| SLPHYIDNPFRQ | 314 |
| SLSKANILHLYG | 315 |
| SLVTADASFTPS | 316 |
| SMAAKSS | 317 |
| SMVYGNRLPSAL | 318 |
| SMYDTHS | 319 |
| SPEMKPR | 320 |
| SPNFSWLPLGTT | 321 |
| SPNLPWSKLSAY | 322 |
| SPNNPRE | 323 |
| SPNNTRE | 324 |
| SPSLMARSSPYW | 325 |
| SQHSTQD | 326 |
| SQTLPYSNAPSP | 327 |
| SRTGAHH | 328 |
| SSHHHRH | 329 |
| SSPPRVY | 330 |
| SSSMAKM | 331 |
| SSTLKTFFGFPD | 332 |
| SSTLKTFFGFPDGGGS | 333 |
| SSTQAHPFAPQL | 334 |
| SSTQVQHTLLQT | 335 |
| SSVPGRP | 336 |
| SSYEYHA | 337 |
| STLASMR | 338 |
| STPNSYSLPQAR | 339 |
| STQAHPW | 340 |
| STSAKHW | 341 |
| STVVMQPPPRPA | 342 |
| SVFLPTRHSPDL | 343 |
| SVQTRPLFHSHF | 344 |
| SVSVGMKPSPRP | 345 |
| SVSVGMNAESXA | 346 |
| SVSVGMNAESYG | 347 |
| SVSVGTEAESXA | 348 |
| SWPLYSRDSGLG | 349 |
| SYIDSMVPSTQT | 350 |
| SYKTTDSDTSPL | 351 |
| SYSQMDPPRSLPGGGS | 352 |
| TAAASNLRAVPP | 353 |
| TAPLSHPPRPGA | 354 |
| TDHPPKA | 355 |
| TGLAKTA | 356 |
| TGLLPNSSGAGI | 357 |
| TGPPSRQPAPLH | 358 |
| TGPTSLS | 359 |
| THPVVFEDERLF | 360 |
| TIHSKPA | 361 |
| TKDWLPS | 362 |
| TLAFQTA | 363 |
| TLAPTFR | 364 |
| TLDKYTRLLSRY | 365 |
| TLGLPML | 366 |
| TLLRTQV | 367 |
| TLMTTPP | 368 |
| TLPSPLALLTVH | 369 |
| TLQRMGQ | 370 |
| TLSNGHRYLELL | 371 |
| TMGFTAPRFPHY | 372 |
| TMRNPITSLISV | 373 |
| TMRNPITSLISVGGGS | 374 |
| TMTNMAK | 375 |
| TPLSYLKGLVTV | 376 |
| TPLTSPSLVRPQ | 377 |
| TPSPKLLQVFQA | 378 |
| TPSTGLGMSPAV | 379 |
| TPVYSLKLGPWP | 380 |
| TQTWPQSSSHGL | 381 |
| TRFYDSL | 382 |
| TRLVPSRYYHHP | 383 |
| TSPIPQMRTVPP | 384 |
| TTKNFNK | 385 |
| TTLSPRT | 386 |
| TTNSSMTMQLQR | 387 |
| TTTLPVQPTLRN | 388 |
| TTTWTTTARWPL | 389 |
| TTYNSPP | 390 |
| TVAQMPPHWQLT | 391 |
| TVLGTFP | 392 |
| TWNSNSTQYGNR | 393 |
| TWTLPAMHPRPA | 394 |
| VHLTHGQ | 395 |
| VHPRPSL | 396 |
| VHTSLLQKHPLP | 397 |
| VLPNIYMTLSA | 398 |
| VMDFASPAHVLP | 399 |
| VNQEYWFFPRRP | 400 |
| VPPISXTFLFXSTXS | 401 |
| VPPLHPALSRXN | 402 |
| VSPFLSPTPLLF | 403 |
| VSRLGTPSMHPS | 404 |
| VVKSNGE | 405 |
| VYSSPLSQLPR | 406 |
| WLPPRTQ | 407 |
| WPANKLSTKSMY | 408 |
| WPFNHFPWWNVP | 409 |
| WPTYLNPSSLKA | 410 |
| WSAHIVPYSHKP | 411 |
| WWPNSLNWVPRP | 412 |
| WYPNHLA | 413 |
| XITXGAY | 414 |
| XPRRAVAAL | 415 |
| XPRRAVAXL | 416 |
| XPRRXVAXL | 417 |
| XXFPLXG | 418 |
| YATQHNWRLKHE | 419 |
| YCPMRLCTDC | 420 |
| YELQMPLTLPLN | 421 |
| YEPAAAE | 422 |
| YGKGFSPYFHVT | 423 |
| YPHYSLPGSSTL | 424 |
| YPIMSHTCCHGV | 425 |
| YPKALRN | 426 |
| YPSLLKMQPQFS | 427 |
| YQPRPFVTTSPM | 428 |
| YSAPLARSNVVM | 429 |
| YTRLSHNPYTLS | 430 |
| YTTHVLPFAPSS | 431 |
| YTWQTIREQYEM | 432 |

In some embodiments, a targeting peptide comprises one or more sequences of Table 6. In some embodiments, the targeting peptide comprises a sequence at least about 70%, 75%, 80%, 85%, 90%, 95%, or 100% identical to a sequence of Table 6.

Additional targeting peptides useful in the present disclosure include any one of SEQ ID NO: 1 to SEQ ID NO: 558 of U.S. Pat. No. 7,572,766, the contents of which is incorporated by reference in its entirety. In some embodiments, the targeting peptide comprises a sequence at least about 70%, 75%, 80%, 85%, 90%, 95%, or 100% identical to any one of SEQ ID NO: 1 to SEQ ID NO: 558 of U.S. Pat. No. 7,572,766.

In some embodiments, the device or kit comprises a chimeric polypeptide comprising the targeting peptide and a targeting moiety. In some cases, the chimeric polypeptide comprises a sequence at least about 70%, 75%, 80%, 85%, 90%, 95%, or 100% identical to SEQ ID NO: 433 (ASGAGGSEGGGSEGGTSGATGAGTSTSGGGASTGGGTGQAKHKQRKRLKSSCKRHPL YVDFSDVGWNDWIVAPPGYHAFYCHGECPFPLADHLNSTNHAIVQTLVNSVNSKIPKA CCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 434 (MPIGSLLADTTHHRPWTVIGESTHHRPWSIIGESSHHKPFTGLGDTTHHRPWGILAESTH HKPWTASGAGGSEGGGSEGGTSGATGAGTSTSGGGASTGGGTGQAKHKQRKRLKSSC KRHPLYVDFSDVGWNDWIVAPPGYHAFYCHGECPFPLADHLNSTNHAIVQTLVNSVNS KIPKACCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 435 (LLADTTHHRPWTVIGESTHHRPWSIIGESSHHKPFTGLGDTTHHRPWGILAESTHHKPW TASGAGGSEGGGSEGGTSGATGAGTSTSGGGASTGGGTGQAKHKQRKRLKSSCKRHPL YVDFSDVGWNDWIVAPPGYHAFYCHGECPFPLADHLNSTNHAIVQTLVNSVNSKIPKA CCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 436 (VIGESTHHRPWSIIGESSHHKPFTGLGDTTHHRPWGILAESTHHKPWTASGAGGSEGGG SEGGTSGATGAGTSTSGGGASTGGGTGQAKHKQRKRLKSSCKRHPLYVDFSDVGWND WIVAPPGYHAFYCHGECPFPLADHLNSTNHAIVQTLVNSVNSKIPKACCVPTELSAISML YLDENEKVVLKNYQDMVVEGCGCR). In some cases, the chimeric polypeptide comprises a sequence at least about 70%, 75%, 80%, 85%, 90%, 95%, or 100% identical to SEQ ID NO: 437 (IIGESSHHKPFTGLGDTTHHRPWGILAESTHHKPWTASGAGGSEGGGSEGGTSGATGA GTSTSGGGASTGGGTGQAKHKQRKRLKSSCKRHPLYVDF SDVGWNDWIVAPPGYHAF YCHGECPFPLADHLNSTNHAIVQTLVNSVNSKIPKACCVPTELSAISMLYLDENEKVVL KNYQDMVVEGCGCR). In some cases, the chimeric polypeptide comprises a sequence at least about 70%, 75%, 80%, 85%, 90%, 95%, or 100% identical to SEQ ID NO: 438 (GLGDTTHHRPWGILAESTHHKPWTASGAGGSEGGGSEGGTSGATGAGTSTSGGGAST GGGTGQAKHKQRKRLKSSCKRHPLYVDFSDVGWNDWIVAPPGYHAFYCHGECPFPLA DHLNSTNHAIVQTLVNSVNSKIPKACCVPTELSAISMLYLDENEKVVLKNYQDMVVEG CGCR). In some cases, the chimeric polypeptide comprises a sequence at least about 70%, 75%, 80%, 85%, 90%, 95%, or 100% identical to SEQ ID NO: 439 (ILAESTHHKPWTASGAGGSEGGGSEGGTSGATGAGTSTSGGGASTGGGTGQAKHKQR KRLKSSCKRHPLYVDFSDVGWNDWIVAPPGYHAFYCHGECPFPLADHLNSTNHAIVQT LVNSVNSKIPKACCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 440 ((X)QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGC R), wherein X comprises a targeting peptide and optionally a linker. For example, the targeting peptide comprises one or more of SEQ ID NOS: 1-41. In some cases, the chimeric polypeptide comprises a sequence at least about 70%, 75%, 80%, 85%, 90%, 95%, or 100% identical to SEQ ID NO: 441 ((X)ASGAGGSEGGGSEGGTSGATGAGTSTSGGGASTGGGTGQAKHKQRKRLKSSCKRH PLYVDFSDVGWNDWIVAPPGYHAFYCHGECPFPLADHLNSTNHAIVQTLVNSVNSKIP KACCVPTELSAISMLYLDENEKVVLKNYQDMVVEGCGCR), wherein X comprises a targeting peptide and optionally a linker. For example, the targeting peptide comprises one or more of SEQ ID NOS: 1-41.

In some embodiments, a therapeutic agent is not connected to a structure using a targeting moiety. For example, the therapeutic agent may interact with the structure via non-covalent bonds. The therapeutic agent may be connected to a structure by hydrogen bonding, ionic bonding, hydrophobic interactions, or van der Waals forces. The therapeutic agent may also be connected to a structure using covalent bonds. Examples of methods for connecting using covalent bonds includes chemical linkers and spacers that are used for modifying active groups within proteins such as amines, thiols and carbohydrates.

Device Manufacture

Further provided herein are methods of manufacturing a device comprising a structure and a therapeutic agent. Some methods comprise: (a) providing a first solution of a therapeutic agent (e.g., a chimeric polypeptide comprising the therapeutic agent and a targeting moiety), (b) providing a 3D structure, and (c) combining (a) and (b). In some embodiments, the therapeutic agent is present in the first solution at a concentration of about 0.25-1.5, 0.25-1.25, 0.25-1, 0.25-0.75, 0.25-0.5, 0.5-1.5, 0.5-1.25, 0.5-1, 0.5-0.75, 0.75-1.5, 0.75-1.25, 0.75-1, 1-1.5, 1-1.25, 1.25-1.5, 0.25, 0.5, 1, 1.25, or 1.5 mg/mL. In some methods, step (c) comprises incubating the first solution and structure for about 10-240, 10-180, 10-120, 20-240, 20-180, 20-120, 30-240, 30-180, 30-120, 40-240, 40-180, 40-120, 50-240, 50-180, 50-120, 60-240, 60-180, 60-120, 70-240, 70-180, 70-120, 80-240, 80-180, 80-120, 90-240, 90-180, 90-120, 100-240, 100-180, 100-120, 110-240, 110-180, 110-120, 10, 20, 30, 40, 50, 60, 70, 80, 90, 100, 110, 120, 130, 140, 150, 160, 170, 180, 190, 200, 210, 220, 230, or 240 minutes. In some methods, step (c) comprises incubating the first solution and structure with movement, such as rotation and/or shaker (e.g., using a plate shaker). In some cases, the first solution comprises a buffer. For example, the buffer comprises sodium acetate and acetic acid. In some cases, the first solution has a pH from about 4 to about 5, or about 4, 4.1, 4.15, 4.2, 4.25, 4.3, 4.35, 4.4, 4.45, 4.5, 4.45, 4.6, 4.65, 4.7, 4.75, 4.8, 4.85, 4.9, 4.95, or 5. In some cases, the first solution comprises a salt. For example, the first solution comprises sodium chloride, such as about 50-150, 50-140, 50-130, 50-120, 50-110, 50-100, 50-90, 50-80, 50-70, 50-60, 60-150, 60-140, 60-130, 60-120, 60-110, 60-100, 60-90, 60-80, 60-70, 70-150, 70-140, 70-130, 70-120, 70-110, 70-100, 70-90, 70-80, 80-150, 80-140, 80-130, 80-120, 80-110, 80-100, 80-90, 90-150, 90-140, 90-130, 90-120, 90-110, 90-100, 100-150, 100-140, 100-130, 100-120, 100-110, 110-150, 110-140, 110-130, 110-120, 120-150, 120-140, 120-130, 130-150, 130-140, or 140-150. In some embodiments, the first solution comprises 10 mM sodium acetate, 7 mM acetic acid, 100 mM NaCl, pH=4.75. In some embodiments, the method further comprises (d) washing the 3D structure of step (c) with a second solution, such as phosphate buffered saline (PBS). In some embodiments, the method further comprises drying the 3D structure of step (c) or step (d).

In some embodiments, the mass of the therapeutic agent (e.g., a therapeutic agent alone or a therapeutic agent connected to a targeting moiety) per cubic centimeter of the structure in a device is between about 0.05 and 50 (mg/cc), e.g., about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50 mg/cc or any number therebetween. For example, the therapeutic agent is about 1, 1.1, 1.2, 1.3, 1.4, 1.5, 1.6, 1.7, 1.8, 1.9, or 2 mg per cubic centimeter device. One method of measuring the amount of therapeutic peptide bound to the structure includes: (1) measuring the mass of therapeutic peptide input in the first solution, (2) measuring the mass of the therapeutic agent remaining in the first solution after combination with and removal from the structure, (3) measuring the mass of the therapeutic agent in the second solution if a wash step is included, (4) summing (2) and (3); and subtracting the sum of (4) from (1).

### Methods of Treatment

In another aspect, provided are methods of treating a subject with a 3D printed structure herein. In some methods, the subject is treated with a device comprising a therapeutic peptide and the structure. In some instances, the subject has a bone fracture or a bone defect. In some instances, the subject requires a vertebral fusion of the spine. In some instances, the subject has a cartilage tear or cartilage defect. In some instances, the subject has cartilage loss.

In some embodiments, the subject is suffering from a defect in bone, cartilage, soft tissue, tendon, fascia, ligament, organ, osteotendinous tissue, dermal, or osteochondral, or a combination of one or more of the aforementioned defects. In some embodiments, a defect is a lack of bone, cartilage, soft tissue, tendon, fascia, ligament, organ, osteotendinous tissue, dermal, or osteochondral, or a combination of one or more of the aforementioned defects. In some embodiments, a defect in the subject arises from trauma. In some embodiments, a defect in the subject arises due to a congenital condition. In some embodiments, a defect in the subject arises due to an acquired condition. In some embodiments, a defect refers to the absence, loss, and/or break in a tissue and/or organ of the body. In some embodiments, a “bone defect” refers to the absence or loss (e.g., partial loss) of bone at an anatomical location in a subject where it would otherwise be present in a control healthy subject. A bone defect may be the result of an infection (e.g., osteomyelitis), a tumor, a trauma, or an adverse event of a treatment. A bone defect may also affect the muscles, soft tissue, tendons, or joints surrounding the bone defect and cause injury. In some embodiments, a bone defect includes damage to a soft tissue. In some embodiments, a “cartilage defect” refers to the absence or loss (e.g., partial loss) of cartilage at an anatomical location in a subject where it would otherwise be present in a control healthy subject. A cartilage defect may be the result of disease, osteochondritis, osteonecrosis, or trauma. For example, a cartilage defect may affect the knee joint.

Non-limiting examples of conditions suitable for treatment with a structure or device described herein include osteoarthritis, disc degeneration, congenital defect, spinal stenosis, spondylolisthesis, spondylosis, bone fracture, scoliosis, kyphosis, spinal fusion (PLF, and interbody fusions), trauma repair of bone, dental repair, craniomaxillofacial repair, ankle fusion, kyphoplasty, balloon osteoplasty, scaphoid facture repair, tendeno-osseous repair, osteoporosis, avascular necrosis, congenital skeletal malformations, costal reconstruction, subchondral bone repair, cartilage repair (e.g., at low doses), or trauma, or a combination thereof. BMP2 is also involved in hair follicle development, therefore the methods may comprise treatment to hair follicles. The trauma may be to the bone, cartilage, soft tissue, tendon, fascia, ligament, organ, osteotendinous tissue, or dermal tissue, or osteochondral tissue. In some embodiments, the method is to treat an osteochondral injury.

The methods of treatment may comprise spinal fusion. In some embodiments, spinal fusion is a surgical technique to join two or more vertebrae. In some embodiments, the spinal fusion comprises PLF. In some embodiments, the spinal fusion comprises interbody fusions.

Provided herein are methods of promoting bone or cartilage formation in a subject in need thereof that include: administering to the subject a therapeutically effective amount of any of the structures or devices described herein. Some embodiments of these methods can further include first selecting a subject in need of bone or cartilage formation. In some embodiments, the structure or device is administered to the subject proximal to the desired site of bone or cartilage formation in the subject.

Also provided herein are methods of replacing and/or repairing bone or cartilage in a subject in need thereof that include: administering to the subject a therapeutically effective amount of any of the structure or devices described herein. Some embodiments of these methods can further include first selecting a subject in need of bone replacement, bone repair, cartilage replacement, or cartilage repair. In some embodiments, the structure or device is administered to the subject proximal to the desired site of bone or cartilage replacement or repair in the subject.

Also provided herein are methods of treating a bone fracture or bone loss in a subject in need thereof, the method comprising administering to the subject a therapeutically effective amount of any of the structure or devices described herein. Some embodiments of these methods can further include first selecting a subject having a bone fracture or bone loss. In some embodiments, the structure or device is administered to the subject proximal to the bone fracture or the site of bone loss in the subject.

Also provided herein are methods of repairing soft tissue in a subject in need thereof that include administering to the subject a therapeutically effective amount of any of the structure or devices described herein. Some embodiments of these methods can further include first selecting a subject having a bone fracture or bone loss. In some embodiments, the composition is administered to the subject proximal to the bone fracture or the site of bone loss in the subject.

Also provided herein are methods of localized delivery of a therapeutic to a subject in need thereof that include: administering to the subject a therapeutically effective amount of any of the structure or devices described herein. Some embodiments of these methods can further include first selecting a subject having a bone fracture or bone loss. In some embodiments, the structure or device is administered to the subject proximal to the bone fracture or the site of bone loss in the subject.

Methods of determining the efficacy of treatment of a bone fracture or bone loss in a subject are known in the art and include, e.g., imaging techniques (e.g., magnetic resonance imaging, X-ray, or computed tomography).

Methods of detecting bone or cartilage formation, or replacement or repair of bone or cartilage in a subject are also known in the art and include, e.g., imaging techniques (e.g., magnetic resonance imaging, X-ray, or computed tomography).

Suitable animal models for treatment of a bone fraction or bone loss, bone or cartilage formation, or bone or cartilage replacement or repair are known in the art. Non-limiting examples of such animal models are described in the Examples and in, e.g., Drosse et al., Tissue Engineering Part C 14(1):79-88, 2008; Histing et al., Bone 49:591-599, 2011; and Poser et al., Hindawi Publishing Corporation, BioMed Research International; Article ID 348635, 2014.

As used herein, a method of treatment comprises administering to the subject a structure or device herein. In some embodiments, administration comprises implanting a polypeptide or composition herein.

In some embodiments, a polypeptide and/or composition herein comprising BMP-2 is administered to the subject. In some embodiments, the BMP2 comprises a sequence at least about 70%, 75%, 80%, 85%, 90%, 95%, or 100% identical to SEQ ID NO: 32. In some embodiments, the BMP-2 is administered to induce formation of bone in the subject. In some embodiments, the BMP-2 is administered to induce formation of cartilage. In some embodiments, the BMP-2 is administered in a spinal fusion.

### FURTHER EMBODIMENTS

Further embodiments include (1) An ink comprising: tricalcium phosphate ceramic (β-TCP) or hydroxyapatite (HA) particles; a biocompatible water-soluble polymer binder; a dispersant; and an anti-foaming agent. (2) The ink of embodiment 1, wherein the biocompatible water-soluble polymer is Pluronic® F-127 polymer, the anti-foaming agent is 1-octanol, and the dispersant is Darvan® 821-A. (3) An ink comprising: tricalcium phosphate ceramic (β-TCP) or HA particles; a biocompatible non-water-soluble polymer; and three or more solvents, wherein each of the solvents has a vapor pressure that is different from the vapor pressure of the other solvents. (4) The ink of embodiment 3, wherein the biocompatible non-water-soluble polymer is polycaprolactone (PCL) and the three or more solvents comprise dichloromethane, 2-butoxyethanol, and dibutyl phthalate. (5) An ink comprising: tricalcium phosphate ceramic (β-TCP) or HA particles; a biocompatible water-soluble polymer; and a biocompatible non-water-soluble polymer. (6) The ink of embodiment 5, wherein the biocompatible non-water-soluble polymer is polycaprolactone (PCL) or poly(lactic-co-glycolic acid) (PLGA) and the biocompatible water-soluble polymer is polyethylene glycol. (7) A three-dimensional implantable object comprising an ink of any of the previous embodiments. (8) The object of embodiment 7, wherein the object is a porous scaffold comprising a plurality of layers, each layer comprising the ink. (9) A method of treating a subject having a tissue defect, the method comprising: surgically implanting the three-dimensional object of embodiment 7 into the tissue defect of the subject, thereby treating the subject. (10) A method of manufacturing an ink for three-dimensional printing, the method comprising: preparing a liquid solution; combining the liquid solution with a portion of calcium phosphate ceramic (β-TCP) or HA particles; and mixing the liquid solution and β-TCP particles via centrifugal mixing. (11) The method of embodiment 10, comprising combining the polymeric solution with a dispersing agent and an antifoaming agent. (12) The method of embodiment 10, comprising combining at least an additional portion of β-TCP or HA particles and repeating the mixing steps at least once. (13) The method of embodiment 10, comprising ensuring that all the β-TCP or hydroxyapatite particles are wet by the liquid solution. (14) A method of preparing a three-dimensional printed implanted object, the method comprising: printing a printed structure using the ink of embodiment 10; drying the printed structure; and heat-treating the printed structure. (15) The method of embodiment 14, wherein heat-treating the printed structure comprises: heating the printed structure so that the polymer is removed from the printed structure; and heating the printed structure to sinter the β-TCP or hydroxyapatite particles. (16) The method of embodiment 15, wherein heat-treating the printed structure comprises: heating the printed structure from room temperature to 600° C. at a heating rate of 1° C./min; heating the printed structure at 600° C. for one hour; heating the printed structure from 600° C. to 1140° C. at a rate of 5° C./min; heating the printed structure at 1140° C. for 4 hours; and cooling the printed structure. (17) The method of embodiment 14, further comprising coating the printed structure with a tetherable protein by soaking the printed structure in a tBMP2 solution. (18) The method of embodiment 14, comprising combining the polymeric solution with three or more solvents, wherein each of the solvents has a vapor pressure that is different from the vapor pressure of the other solvents. (19) The method of embodiment 18, the method comprising: printing a printed structure using the ink of embodiment 18; drying the printed structure to remove a first of the solvents; soaking the printed structure in a mixture of water and ethanol such that a second one of the solvents is removed from the printed structure; and soaking the printed structure in a mixture of water such that a third one of the solvents is removed from the printed structure. (20) A method of manufacturing an ink for three-dimensional printing, the method comprising: preparing a polymeric power mixture including tricalcium phosphate ceramic particles (β-TCP or hydroxyapatite), a biocompatible water-soluble polymer, and a biocompatible non-water-soluble polymer; mixing the powder mixture via centrifugal mixing to at least partially melt the powders; and heating the powder mixture in an extruder chamber of a 3D printer to melt the powders prior to printing. (21) A method of preparing a three-dimensional printed implanted object, the method comprising: printing a printed structure using the ink of embodiment 20; and soaking the 3D printed structure in water to remove the water-soluble polymer. (22) The method of embodiment 21, further comprising coating the printed structure with a tetherable protein by soaking the printed structure in a tBMP2 solution. (23) A method of treating a subject having a tissue defect, the method comprising: coating β-TCP or HA granules with tBMP2; mixing a sodium carboxymethylcellulose hydrogel with the granules; and mixing the mixture via centrifugal mixing to create a putty material; and surgically implanting the putty material into the tissue defect of the subject, thereby treating the subject. (24) A method of preparing a bone implant, comprising: forming an ink by mixing a light-sensitive resin with β-TCP or HA particles, a photocurable acrylate, plasticizer, dispersant, photoinitiator, and photoabsorber; forming an implantable object with the ink; and coating the implantable object with a tetherable protein to form the bone implant. (25) The method of embodiment 24, further comprising heat-treating the implantable object. (26) The method of embodiment 24, wherein forming the implantable object comprises digital light processing of the ink.

Other ways of demonstrating the utility of this invention is by demonstrating bone regeneration in a lumbar spinal fusion indication (a 3D printed insert for spinal fusion cage), tibial segmental defects (a 3D printed scaffold based on patient CT data), and alveolar ridge augmentation (a 3D printed thin barrier membrane).

The terminology used herein is for the purpose of describing particular cases only and is not intended to be limiting. The singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The term “subject” as used herein refers to any mammal. A subject therefore refers to, for example, mice, rats, dogs, cats, horses, cows, pigs, guinea pigs, rats, humans, monkeys, and the like. When the subject is a human, the subject may be referred to herein as a patient. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon), a human, or a rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine, or primate animals) may be employed.

In some embodiments, the term “therapeutically effective amount” refers to an amount of a polypeptide or composition effective to “treat” a disease, condition or disorder in a subject. In some cases, therapeutically effective amount of the polypeptide or composition reduces the severity of symptoms of the disease, condition or disorder. In some instances, the disease, condition or disorder comprises a defect in an organ or tissue.

“Affinity” refers to the strength of the sum total of non-covalent interactions between a 0-TCP binding sequence (or a chimeric polypeptide or polypeptide comprising a β-TCP binding sequence) and its binding partner (e.g., β-TCP). Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining affinity are known in the art.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Each of the embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

A number of embodiments of the disclosure have been described. Nevertheless, it will be understood that various modifications may be made without departing from the spirit and scope of the disclosure. For example, cells can be seeded within the implantable structures. These cells can include osteocytes or other bone cells, chondrocytes, and/or meniscal cells. In some instances, the cells can be added to the completed implantable structures. Additionally, while specific formulations for the inks are described, variations of the specific quantities of each ink ingredient are possible. Accordingly, other embodiments are within the scope of the following claims.

### Embodiments

Embodiment 1: A device comprising: a therapeutic agent non-covalently bound to a printed three-dimensional structure, the printed three-dimensional structure comprising about 50% to about 100% by weight ceramic and about 0% to about 50% by weight polymer.
Embodiment 2: The device of embodiment 1, wherein the three-dimensional structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.
Embodiment 3: The device of embodiment 1 or embodiment 2, wherein the ceramic comprises calcium phosphate, hydroxyapatite, fluorapatite, bone, silicate, or vanadate, or a combination thereof.
Embodiment 4: The device of embodiment 1 or embodiment 2, wherein the ceramic comprises beta-tricalcium phosphate (β-TCP).
Embodiment 5: The device of any one of embodiments 1-4, comprising the polymer, wherein the polymer comprises polycaprolactone.
Embodiment 6: The device of embodiment 1 or embodiment 2, comprising about 100% by weight ceramic.
Embodiment 7: The device of embodiment 6, wherein the ceramic comprises beta-tricalcium phosphate (β-TCP).
Embodiment 8: The device of embodiment 1 or embodiment 2, comprising about 70% to about 80% by weight ceramic, and about 20% to about 30% by weight polymer.
Embodiment 9: The device of embodiment 8, wherein the ceramic comprises beta-tricalcium phosphate (β-TCP) and the polymer comprises polycaprolactone.
Embodiment 10: The device of any one of embodiments 1-9, wherein the printed three-dimensional structure is formed from an ink comprising about 30% to about 70% by weight the ceramic, about 5% to about 30% by the weight polymer, and optionally an anti-foaming agent and/or a dispersing agent.
Embodiment 11: The device of any one of embodiments 1-10, wherein the therapeutic agent comprises a mammalian growth factor or a functional portion thereof.
Embodiment 12: The device of any one of embodiments 1-10, wherein the therapeutic agent comprises one or more polypeptides selected from Table 4, or a functional portion thereof.
Embodiment 13: The device of any one of embodiments 1-10, wherein the therapeutic agent comprises a bone morphogenetic protein (BMP).
Embodiment 14: The device of any one of embodiments 1-13, wherein the therapeutic agent comprises a targeting moiety, and the targeting moiety is non-covalently bound to the printed three-dimensional structure.
Embodiment 15: The device of embodiment 14, wherein the targeting moiety is bound to the printed three-dimensional structure with an affinity of about 1 pM to about 100 μm.
Embodiment 16: The device of embodiment 14 or embodiment 15, wherein the targeting moiety comprises a polypeptide at least about 70%, 75%, 80%, 85%, 90%, 95%, or 100% identical to any one of the sequences of Tables 5-6.
Embodiment 17: The device of embodiment 14 or embodiment 15, wherein the targeting moiety comprises about 2, 3, 4, 5, 6, 7, 8, 9, or 10 sequences selected from the sequence of Tables 5-6.
Embodiment 18: The device of any one of embodiments 1-17, wherein the therapeutic agent is a chimeric polypeptide comprising a sequence at least about 70%, 75%, 80%, 85%, 90%, 95%, or 100% identical to any one of SEQ ID NOS: 794-802.
Embodiment 19: A method of treating a condition in a subject in need thereof, the method comprising administering to the subject the device of any one of embodiments 1-18.
Embodiment 20: The method of embodiment 19, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.
Embodiment 21: The method of embodiment 19 or embodiment 20, wherein the method comprises spinal fusion.
Embodiment 22: The method of embodiment 21, wherein the spinal fusion comprises posterior lumbar fusion (PLF) and/or interbody fusion.
Embodiment 23: The method of embodiment 19 or embodiment 20, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.
Embodiment 24: A method of manufacturing a three-dimensional structure, the method comprising: providing a solution comprising a ceramic, a polymer, and optionally an anti-foaming agent and/or dispersing agent, mixing the solution to obtain an ink formulation, and depositing the ink formulation in a three-dimensional form; wherein: (i) the ink formulation comprises about 30% to about 70% by weight ceramic and about 5% to about 60% by weight polymer, and/or (ii) the three-dimensional structure comprises about 50% to about 100% by weight ceramic and about 0% to about 50% by weight polymer.
Embodiment 25: The method of embodiment 24, wherein the ceramic of the ink formulation and/or three-dimensional structure comprises calcium phosphate, hydroxyapatite, fluorapatite, bone, silicate, or vanadate, or a combination thereof.
Embodiment 26: The method of embodiment 24, wherein the ceramic of the ink formulation and/or three-dimensional structure comprises beta-tricalcium phosphate (β-TCP).
Embodiment 27: The method of any one of embodiments 24-26, wherein the polymer of the ink formulation comprises a first polymer comprising polycaprolactone and a second polymer comprising polyethylene glycol.
Embodiment 28: The method of embodiment 27, wherein the ink formulation comprises about 10% to about 30% by weight polycaprolactone and about 10% to about 30% by weight polyethylene glycol.
Embodiment 29: The method of any one of embodiments 24-28, wherein the three-dimensional structure comprises about 100% by weight ceramic.
Embodiment 30: The method of any one of embodiments 24-28, wherein the three-dimensional structure comprises about 100% by weight beta-tricalcium phosphate (β-TCP).
Embodiment 31: The method of any one of embodiments 24-28, wherein the three-dimensional structure comprises about 70% to about 80% by weight ceramic, and about 20% to about 30% by weight polymer.
Embodiment 32: The method of any one of embodiments 24-28, wherein the three-dimensional structure comprises about 70% to about 80% by weight beta-tricalcium phosphate (β-TCP), and about 20% to about 30% by weight polycaprolactone.
Embodiment 33: The method of any one of embodiments 24-32, further comprising combining the three-dimensional structure with a therapeutic agent.
Embodiment 34: The method of embodiment 33, wherein the therapeutic agent comprises a mammalian growth factor or a functional portion thereof.
Embodiment 35: The method of embodiment 33, wherein the therapeutic agent comprises one or more polypeptides selected from Table 4, or a functional portion thereof.
Embodiment 36: The method of embodiment 33, wherein the therapeutic agent comprises a bone morphogenetic protein (BMP).
Embodiment 37: The method of any one of embodiments 33-36, wherein the therapeutic agent comprises a targeting moiety that non-covalently binds to the three-dimensional structure.
Embodiment 38: The method of embodiment 37, wherein the targeting moiety binds to the printed three-dimensional structure with an affinity of about 1 pM to about 100 μm.
Embodiment 39: The method of embodiment 37 or embodiment 38, wherein the targeting moiety comprises a polypeptide at least about 70%, 75%, 80%, 85%, 90%, 95%, or 100% identical to any one of the sequences of Tables 5-6.
Embodiment 40: The method of embodiment 37 or embodiment 38, wherein the targeting moiety comprises about 2, 3, 4, 5, 6, 7, 8, 9, or 10 sequences selected from the sequences of Tables 5-6.
Embodiment 41: The method of any one of embodiments 33-40, wherein the therapeutic agent is a chimeric polypeptide comprising a sequence at least about 70%, 75%, 80%, 85%, 90%, 95%, or 100% identical to any one of SEQ ID NOS: 794-802.
Embodiment 42: A method of treating a condition in a subject in need thereof, the method comprising administering to the subject the three-dimensional structure manufactured by the method of any one of embodiments 24-41.
Embodiment 43: The method of embodiment 42, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.
Embodiment 44: The method of embodiment 42 or embodiment 43, wherein the method comprises spinal fusion.
Embodiment 45: The method of embodiment 44, wherein the spinal fusion comprises posterior lumbar fusion (PLF) and/or interbody fusion.
Embodiment 46: The method of embodiment 42 or embodiment 43, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.
Embodiment 47: An ink formulation for three-dimensional printing, the formulation comprising about 30% to about 70% by weight ceramic, and about 5% to about 30% by weight polymer.
Embodiment 48: The ink formulation of embodiment 47, wherein the ceramic comprises calcium phosphate, hydroxyapatite, fluorapatite, bone, silicate, or vanadate, or a combination thereof.
Embodiment 49: The ink formulation of embodiment 47 or embodiment 48, wherein the ceramic comprises beta-tricalcium phosphate (β-TCP).
Embodiment 50: The ink formulation of any one of embodiments 47-49, comprising about 50% to about 70% by weight ceramic, about 10% to about 30% by weight a first polymer, and about 10% to about 30% by weight a second polymer.
Embodiment 51: The ink formulation of embodiment 47, comprising about 50% to about 70% by weight beta-tricalcium phosphate (β-TCP), about 10% to about 30% by weight a first polymer comprising polycaprolactone, and about 10% to about 30% by weight a second polymer comprising polyethylene glycol.
Embodiment 52: The ink formulation of any one of embodiments 47-49, comprising about 50% to about 70% by weight ceramic, about 5% to about 15% by weight polymer, and optionally an anti-foaming agent and/or a dispersing agent.
Embodiment 53: The ink formulation of embodiment 47, comprising about 50% to about 70% by weight tricalcium phosphate, about 5% to about 15% by weight poloxamer, and optionally an anti-foaming agent and/or a dispersing agent.
Embodiment 54: The ink formulation of embodiment 52 or embodiment 53, comprising about 0.1% to about 1% by weight anti-foaming agent, wherein the anti-foaming agent optionally comprises an alcohol.
Embodiment 55: The ink formulation of any one of embodiments 52-54, comprising about 0.1% to about 1% by weight dispersing agent, wherein the dispersing agent optionally comprises ammonium polyacrylate.
Embodiment 56: The ink formulation of any one of embodiments 47-49, comprising about 40% to about 60% by weight ceramic, about 5% to about 15% by weight polymer, and about 30% to about 40% by weight solvent.
Embodiment 57: The ink formulation of embodiment 47, comprising about 40% to about 60% by weight beta-tricalcium phosphate (β-TCP), about 5% to about 15% by weight polycaprolactone, and about 30% to about 40% by weight solvent.
Embodiment 58: The ink formulation of embodiment 56 or embodiment 57, wherein the solvent comprises dichloromethane, 2-butoxyethanol, dibutyl phthalate, or chloroform, or a combination thereof.
Embodiment 59: A method of preparing a three-dimensional structure, the method comprising using the formation of any one of embodiments 47-58 as an ink in a three-dimensional printing method.
Embodiment 60: A three-dimensional structure prepared using the ink formulation of any one of embodiments 47-58.
Embodiment 61: The three-dimensional structure of embodiment 60, comprising about 50% to about 100% by weight ceramic.
Embodiment 62: The three-dimensional structure of embodiment 60, comprising about 50% to about 100% by weight tricalcium phosphate.
Embodiment 63: The three-dimensional structure of embodiment 60, comprising about 50% to about 90% by weight tricalcium phosphate and about 10% to about 50% polymer.
Embodiment 64: The three-dimensional structure of embodiment 63, wherein the polymer comprises polycaprolactone.
Embodiment 65: The three-dimensional structure of any one of embodiments 60-64, wherein the structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.
Embodiment 66: A three-dimensional structure comprising about 50% to about 100% by weight ceramic, and about 0% to about 50% polymer.
Embodiment 67: The three-dimensional structure of embodiment 66, wherein the ceramic comprises calcium phosphate, hydroxyapatite, fluorapatite, bone, silicate, or vanadate, or a combination thereof.
Embodiment 68: The three-dimensional structure of embodiment 66 or embodiment 67, wherein the ceramic comprises beta-tricalcium phosphate (β-TCP).
Embodiment 69: The three-dimensional structure of embodiment 66 or embodiment 67, comprising about 50% to about 100% by weight ceramic.
Embodiment 70: The three-dimensional structure of embodiment 66 or embodiment 67, comprising about 100% by weight ceramic.
Embodiment 71: The three-dimensional structure of embodiment 66, comprising about 100% by weight tricalcium phosphate.
Embodiment 72: The three-dimensional structure of embodiment 66 or embodiment 67, comprising about 50% to about 90% by weight ceramic and about 10% to about 50% polymer.
Embodiment 73: The three-dimensional structure of embodiment 66 or embodiment 67, comprising about 50% to about 90% by weight tricalcium phosphate and about 10% to about 50% polymer.
Embodiment 74: The three-dimensional structure of embodiment 72 or embodiment 73, wherein the polymer comprises polycaprolactone.
Embodiment 75: The three-dimensional structure of any one of embodiments 66-74, wherein the structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.
Embodiment 76: The three-dimensional structure of any one of embodiments 66-75, prepared by three-dimensional printing methods.
Embodiment 77: A method of delivering a therapeutic agent to a subject in need thereof, the method comprising delivering to an organ or tissue of the subject a device comprising a therapeutic agent and the three-dimensional structure of any one of embodiments 60-76.
Embodiment 78: A device comprising a therapeutic agent and the three-dimensional structure of any one of embodiments 60-76.
Embodiment 79: The method of embodiment 77 or the device of embodiment 78, wherein the therapeutic agent comprises a mammalian growth factor or functional portion thereof.
Embodiment 80: The method of embodiment 77 or the device of embodiment 78, wherein the therapeutic agent comprises one or more polypeptides selected from Table 4, or a functional portion thereof.
Embodiment 81: The method of embodiment 77 or the device of embodiment 78, wherein the therapeutic agent comprises a bone morphogenetic protein (BMP).
Embodiment 82: The method of any one of embodiments 77 or 79-81, or the device of any one of embodiments 78-81, wherein the device comprises a targeting moiety.
Embodiment 83: The method of embodiment 82 or the device of embodiment 82, wherein the targeting moiety comprises a polypeptide comprising one or more sequences at least about 70%, 75%, 80%, 85%, 90%, 95%, or 100% identical to any one of the sequences of Tables 5-6.
Embodiment 84: The method of embodiment 82 or the device of embodiment 82, wherein the targeting moiety comprises about 2, 3, 4, 5, 6, 7, 8, 9, or 10 sequences selected from the sequences of Tables 5-6.
Embodiment 85: The method of any one of embodiments 82-84 or the device of any one of embodiments 82-84, wherein the targeting moiety non-covalently binds to the three-dimensional structure.
Embodiment 86: The method of any one of embodiments 82-85 or the device of any one of embodiments 82-85, wherein the targeting moiety is connected to the therapeutic agent in a chimeric polypeptide.
Embodiment 87: The method of embodiment 86 or the device of embodiment 86, wherein the chimeric polypeptide comprises a sequence at least about 70%, 75%, 80%, 85%, 90%, 95%, or 100% identical to any one of SEQ ID NOS: 794-802.
Embodiment 88: A method of preparing the device of any one of embodiments 78-87, the method comprising combining the therapeutic agent and the three-dimensional structure, where the therapeutic agent non-covalently binds to the three-dimensional structure.
Embodiment 89: A method of treating a condition in a subject in need thereof, the method comprising administering to the subject the three-dimensional structure of any one of embodiments 66-76, or the device of any one of embodiments 78 or 80-87.
Embodiment 90: The method of embodiment 89, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.
Embodiment 91: The method of embodiment 89 or embodiment 90, wherein the method comprises spinal fusion.
Embodiment 92: The method of embodiment 91, wherein the spinal fusion comprises posterior lumbar fusion (PLF) and/or interbody fusion.
Embodiment 93: The method of embodiment 89 or embodiment 90, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.
Embodiment 94: The device of any one of embodiments 1 to 18 or 78 to 87, the method of any one of embodiments 19 to 46, 77, or 79 to 93, or the three-dimensional structure of any one of embodiments 60 to 76, wherein the three-dimensional structure has a density of between about 1 g/cm³ and about 3 g/cm³.
Embodiment 95: The device of any one of embodiments 1 to 18, 78 to 87, 94, the method of any one of embodiments 19 to 46, 77, or 79 to 94, or the three-dimensional structure of any one of embodiments 60 to 76, or 94 wherein the three-dimensional structure has an open porosity of between about 15% and about 45%.
Embodiment 96: The device of any one of embodiments 1 to 18, 78 to 87, 94, or 95, the method of any one of embodiments 19 to 46, 77, or 79 to 95, or the three-dimensional structure of any one of embodiments 60 to 76, 94, or 95, wherein the three-dimensional structure has a specific surface area of between about 0.50 m²/g and about 1.0 m²/g.
Embodiment 97: The device of any one of embodiments 1 to 18, 78 to 87, or 94 to 96 the method of any one of embodiments 19 to 46, 77, or 79 to 96, or the three-dimensional structure of any one of embodiments 60 to 76 or 94 to 96, wherein the three-dimensional structure has a fiber diameter of about 325 μm and about 475 μm.
Embodiment 98: The device of any one of embodiments 1 to 18, 78 to 87, or 94 to 97, the method of any one of embodiments 19 to 46, 77, or 79 to 97, or the three-dimensional structure of any one of embodiments 60 to 76 or 94 to 97, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.
Embodiment 99: The device of any one of embodiments 1 to 18, 78 to 87, or 94 to 98, the method of any one of embodiments 19 to 46, 77, or 79 to 98, or the three-dimensional structure of any one of embodiments 60 to 76 or 94 to 98, wherein the three-dimensional structure has a density of about 2.44 g/cm³, open porosity of about 19.6%, and a fiber diameter of about 384 μm.
Embodiment 100: The device of any one of embodiments 1 to 18, 78 to 87, or 94 to 98, the method of any one of embodiments 19 to 46, 77, or 79 to 98, or the three-dimensional structure of any one of embodiments 60 to 76 or 94 to 98, wherein the three-dimensional structure has a density of about 1.32 g/cm³, open porosity of about 38%, and a fiber diameter of about 394 μm.
Embodiment 101: The device of any one of embodiments 1 to 18, 78 to 87, or 94 to 98, the method of any one of embodiments 19 to 46, 77, or 79 to 98, or the three-dimensional structure of any one of embodiments 60 to 76 or 94 to 98, wherein the three-dimensional structure has a density of about 1.49 g/cm³, open porosity of about 31%, specific surface area of 0.81 m²/g, and a fiber diameter of about 420 μm.

### Examples

### Example 1: Ink Formulation

An ink formulation comprising 60% by weight β-TCP Powder (spray-dried powder, 10-38 micron particle size), 20% polycaprolactone (50,000 MW, fine powder, Tmelt=60° C.), and 20% by weight polyethylene glycol (1,500 MW flake, Tmelt=60° C.) was prepared. To make a 3.2 cc batch of the ink, 2.062 g of β-TCP powder, 2.062 g of PCL powder, and 0.884 g of polyethylene glycol flake were added to a container. The container was placed in a mixer and the powders were mixed in a FlackTek Speedmixer at 300 rpm for 2 min to homogenize the powder blend. Higher rpm mixing was then carried out for an additional 5 min at 3500 rpm to melt the powders. During this mixing, the internal friction caused the PCL and polyethylene glycol to melt, changing the powders to a viscous molten liquid that was then used as the input for 3D manufacturing of a β-TCP/PCL structure.

### Example 2: Manufacture by 3D Printing

The ink of example 1 was manufactured into a structure using melt-extrusion printing with an Allevi 3 Bioprinter. Briefly, the ink was fitted into a 5 cc stainless steel syringe with a 400 micron inner diameter conical metallic Luer lock tip. Prior to printing, the extruder chamber having the stainless-steel syringe was heated to ensure melting of the ink. The ink was extruded using 120° C. extruder temperature, 70 psi pressure and 5 mm/s-10 mm/s tip velocity. The printed structure was soaked overnight in distilled water to dissolve the polyethylene glycol from the printed material, creating a porous and flexible β-TCP/PCL structure.

The structure was tested using Brunauer-Emmett-Teller (BET) surface area analysis by gas physisorption. The surface area was 0.81 m²/g.

A compression test was performed on a 1 cm diameter×0.75 cm height cylinder of the structure. No rupture was observed at 33% strain, and the structure elastically recovered 10% strain. The elastic modulus was measured as 123 MPa+16 MPa.

### Example 3: Therapeutic Agent

A chimeric polypeptide comprising the BMP therapeutic peptide connected to five beta-tricalcium phosphate binding peptides was expressed and purified using standard expression and purification methods. The chimeric polypeptide is referred to as tBMP-2 and has the following sequence:

(SEQ ID NO: 434)
MPIGSLLADTTHHRPWTVIGESTHHRPWSIIGESSHHKPFTGLGDTTHH
RPWGILAESTHHKPWTASGAGGSEGGGSEGGTSGATGAGTSTSGGGAST
GGGTGQAKHKQRKRLKSSCKRHPLYVDFSDVGWNDWIVAPPGYHAFYCH
GECPFPLADHLNSTNHAIVQTLVNSVNSKIPKACCVPTELSAISMLYLD
ENEKVVLKNYQDMVVEGCGCR.

### Example 4: Device Manufacture

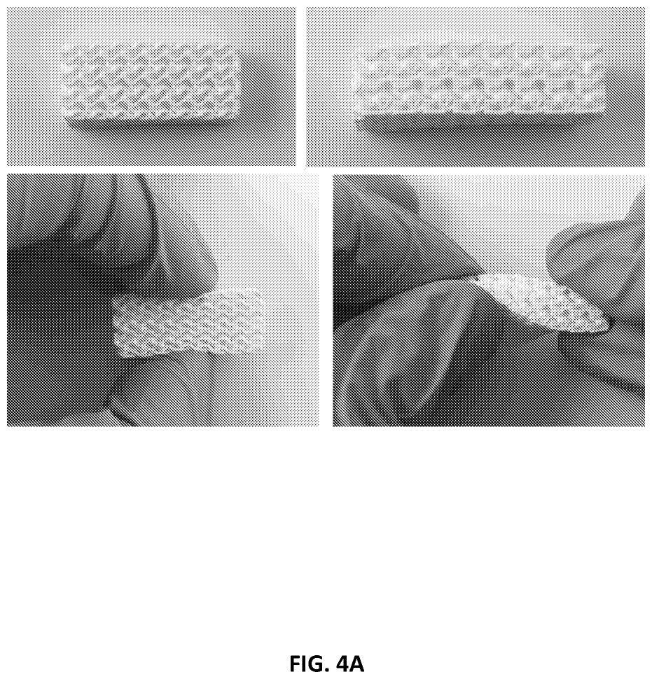

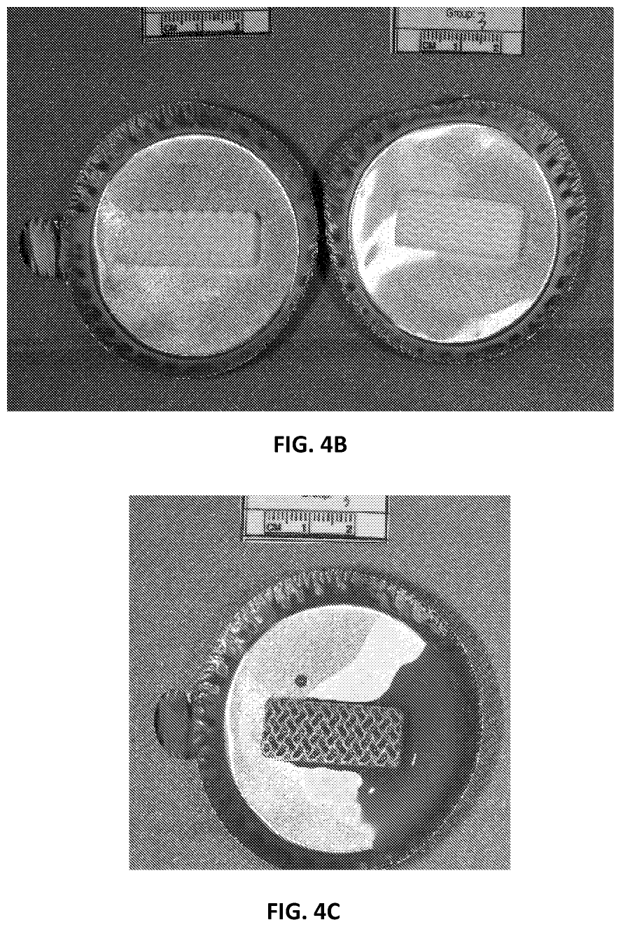

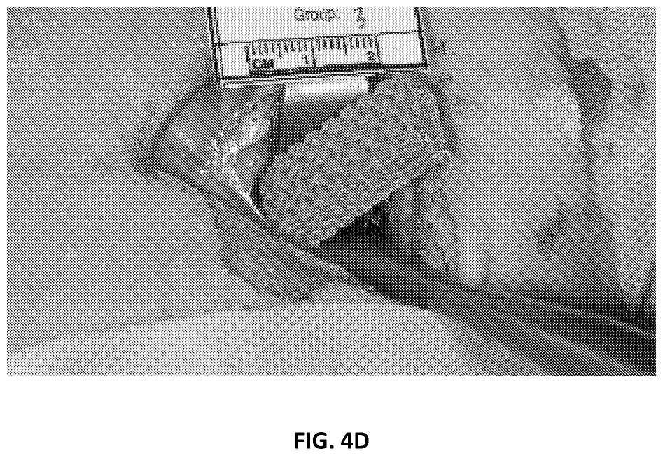

The structure of Example 2 was combined with the tBMP-2 therapeutic agent of Example 3 to create a device. 0.75 mg/mL tBMP-2 binding solution (10 mM sodium acetate, 7 mM acetic acid, 100 mM NaCl, pH=4.75) was prepared and sterilized with 0.22 μm filter. In biosafety cabinet, 8 mL of tBMP-2 binding solution was added to a sterile 15 mL conical tube with sterile pipette. A sterile scaffold from Example 2 was added to binding solution with sterile tweezers, then conical tube was closed and wrapped with parafilm. The tube was placed on a LabLine Instruments Titer Plate Shaker, set at speed to 2, and shaken for 2 hours. In a biosafety cabinet, the scaffold+tBMP-2 was removed with sterile tweezers and placed in a different sterile 15 mL conical tube filled with 8 mL of sterile PBS. The lid was closed, wrapped with parafilm, and returned to the Titer Plate Shaker to shake for 3 minutes at speed 2. The tube was opened in the biosafety cabinet, the scaffold+tBMP-2 was removed with sterile tweezers, and placed in a sterile petri dish. The scaffold+tBMP-2 was allowed to dry overnight in the biosafety cabinet, resulting in the tBMP-2 device. The tBMP-2 device is shown in FIG. 4A.

The mass of tBMP-2 remaining in the binding solution and mass of tBMP-2 in the PBS wash solution was measuring using A280 absorbance measurements. The sum of these masses was calculated and then subtracted from the initial mass of tBMP-2 in binding solution to arrive at the mass of tBMP-2 which remains bound to the 3D printed scaffold. In this example, the scaffold has a tBMP-2 dose of 1.4 mg/cubic centimeter.

### Example 5: Rabbit Model of Posterolateral (PLF) Spine Fusion

The purpose of this experiment was to compare the fusion rates and the degree of new bone formation between autograft versus test article in a rabbit model of posterolateral (PLF) spine fusion.

The rabbit was chosen for this study because it is a commonly used species for nonclinical toxicity and orthopedic implant evaluations, including spine fusion applications. The FDA Guidance document (“Class II Special Controls Guidance Document: Resorbable Calcium Salt Bone Void Filler Device, Guidance for Industry and FDA,” US Food and Drug Administration, Center for Devices and Radiological Health, Jun. 2, 2003) requires preclinical studies to support marketing applications. The rabbit is an approved animal species for spine fusion studies according to ISO 10993-6, Annex D, and the ASTM F3207-17—Standard Guide for in vivo Evaluation of Rabbit Lumbar Inter-transverse Process Spinal Fusion Model. The total number of animals assigned to this study as well as the group size and number of groups is the minimum required to properly characterize efficacy of the test articles.

The test animals were New Zealand White female rabbits, approximately 6-7 months old and approximately 3.5 to 5.0 kgs at surgery. The rabbits were acclimated to the facility for a minimum of 7 days and examined to ensure they were free of clinical signs of disease.

Test Materials

The test material was the tBMP-2 coated device as described in Example 4.

The rabbits were divided into 2 groups, with 3 rabbits in each group. Group 1 was the autograft control, and group 2 received test material.

Pre-Operative Animal Preparation

A fentanyl transdermal patch (25 1.1 g/hr) was placed on the animal prior to surgery. Anesthesia induction was initiated by an injection of acepromazine (0.25-0.75 mg/kg) and butorphanol (approximately 0.5 mg/kg) given IM. An intravenous catheter was placed in the marginal ear vein and general anesthesia was induced using propofol (5-7 mg/kg) to effect IV. After induction of anesthesia, an endotracheal tube was placed and anesthesia maintained using isoflurane (0.54% to effect) in oxygen. Perioperative cefazolin (approximately 40 mg/kg) was administered IV during pre-operative preparation. Eye ointment was be administered to the eyes to prevent corneal drying. Pre-operative radiography was utilized to image the lumbar spine to mark the position of L4 and L5. The dorsal lumbosacral area was clipped and prepared for aseptic surgery by a povidone iodine antiseptic scrub followed by a 70% isopropyl alcohol rinse, repeated three (3) times. The area was painted with povidone iodine solution and draped for aseptic surgery. The animal was transferred to the operating room, positioned on a heated surgery table, connected to the anesthesia machine and monitors, and draped for aseptic surgery. Lactated Ringer's Solution was administered IV during the procedure at approximately 10 ml/kg/hr. Animals were monitored by trained personnel while under general anesthesia to include assessing and documenting the heart rate, respiratory rate, and oxygen saturation percentage every 10-15 minutes.

Surgical Procedure

Autograft Harvest: A midline skin incision was be created over the caudal lumbosacral spine to approach the L4-5 interspaces and the iliac crests. Ropivacaine or other local analgesic agent was infiltrated into the soft tissue adjacent to the crests to provide local analgesia. Approximately 3 cc of iliac crest bone graft (ICBG) was harvested with an osteotome, curette and Rongeur forceps and morselized to irregular sized particles up to 4 mm diameter. ICBG was harvested bilaterally from Group 1 animals and loaded into two open barrel 3 cc syringes.

Spine Fusion: Paraspinal incisions were created in the fascia over the L4-L5 interspaces and the seam between the paraspinalis and multifidus muscles was identified and separated by blunt dissection. Soft tissue was elevated and retracted to expose the dorsal surfaces of L4 and L5 transverse processes bilaterally. Pressure and focal electrocautery were used to control hemorrhage if appropriate. A motorized burr was used to decorticate approximately 2 cm of the dorsal surfaces of the transverse processes of L4 and L5 adjacent to the laminae but not extending onto the pars. Implants were deployed such that they were in contact with and span the distance between the decorticated L4 and L5 transverse processes bilaterally. Surgical incisions were closed in three layers in accordance with standard surgical techniques. See FIG. 4D.

Animal Care

Animals were observed at least twice daily to assess anesthesia recovery, appetite and analgesic efficacy for the first three days after surgery. Thereafter, animals were observed twice daily for general health and well-being and appetite recovery until animals were eating normally. Animals were thereafter observed each morning for general health and well-being and a cursory check performed each afternoon. Rabbits were weighed approximately 7-10 days after surgery at staple removal and at approximately 2-week intervals during the course of the study to monitor general health.

What is claimed is:

1. A device comprising: a therapeutic agent non-covalently bound to a printed three-dimensional structure, the printed three-dimensional structure comprising about 50% to about 100% by weight ceramic and about 0% to about 50% by weight polymer.

2. The device of claim 1, wherein the three-dimensional structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

3. The device of claim 1 or claim 2, wherein the ceramic comprises calcium phosphate, hydroxyapatite, fluorapatite, bone, silicate, or vanadate, or a combination thereof.

4. The device of claim 1 or claim 2, wherein the ceramic comprises beta-tricalcium phosphate (β-TCP).

5. The device of claim 1 or claim 2, comprising the polymer, wherein the polymer comprises polycaprolactone.

6. The device of claim 1 or claim 2, comprising about 100% by weight ceramic.

7. The device of claim 6, wherein the ceramic comprises beta-tricalcium phosphate (β-TCP).

8. The device of claim 1 or claim 2, comprising about 70% to about 80% by weight ceramic, and about 20% to about 30% by weight polymer.

9. The device of claim 8, wherein the ceramic comprises beta-tricalcium phosphate (β-TCP) and the polymer comprises polycaprolactone.

10. The device of claim 1 or claim 2, wherein the printed three-dimensional structure is formed from an ink comprising about 30% to about 70% by weight the ceramic, about 5% to about 30% by the weight polymer, and optionally an anti-foaming agent and/or a dispersing agent.

11. The device of claim 1 or claim 2, wherein the therapeutic agent comprises a mammalian growth factor or a functional portion thereof.

12. The device of claim 1 or claim 2, wherein the therapeutic agent comprises one or more polypeptides selected from Table 4, or a functional portion thereof.

13. The device of claim 1 or claim 2, wherein the therapeutic agent comprises a bone morphogenetic protein (BMP).

14. The device of claim 1 or claim 2, wherein the therapeutic agent comprises a targeting moiety, and the targeting moiety is non-covalently bound to the printed three-dimensional structure.

15. The device of claim 14, wherein the targeting moiety is bound to the printed three-dimensional structure with an affinity of about 1 pM to about 100 μm.

16. The device of claim 14, wherein the targeting moiety comprises a polypeptide at least about 70%, 75%, 80%, 85%, 90%, 95%, or 100% identical to any one of the sequences of Tables 5-6.

17. The device of claim 14, wherein the targeting moiety comprises about 2, 3, 4, 5, 6, 7, 8, 9, or 10 sequences selected from the sequence of Tables 5-6.

18. The device of claim 1 or claim 2, wherein the therapeutic agent is a chimeric polypeptide comprising a sequence at least about 70%, 75%, 80%, 85%, 90%, 95%, or 100% identical to any one of SEQ ID NOS: 794-802.

19. A method of treating a condition in a subject in need thereof, the method comprising administering to the subject the device of claim 1 or claim 2.

20. The method of claim 19, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.

21. The method of claim 19, wherein the method comprises spinal fusion.

22. The method of claim 21, wherein the spinal fusion comprises posterior lumbar fusion (PLF) and/or interbody fusion.

23. The method of claim 19, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

24. A method of manufacturing a three-dimensional structure, the method comprising: providing a solution comprising a ceramic, a polymer, and optionally an anti-foaming agent and/or dispersing agent, mixing the solution to obtain an ink formulation, and depositing the ink formulation in a three-dimensional form; wherein: (i) the ink formulation comprises about 30% to about 70% by weight ceramic and about 5% to about 60% by weight polymer, and/or (ii) the three-dimensional structure comprises about 50% to about 100% by weight ceramic and about 0% to about 50% by weight polymer.

25. The method of claim 24, wherein the ceramic of the ink formulation and/or three-dimensional structure comprises calcium phosphate, hydroxyapatite, fluorapatite, bone, silicate, or vanadate, or a combination thereof.

26. The method of claim 24, wherein the ceramic of the ink formulation and/or three-dimensional structure comprises beta-tricalcium phosphate (β-TCP).

27. The method of claim 24 or claim 25, wherein the polymer of the ink formulation comprises a first polymer comprising polycaprolactone and a second polymer comprising polyethylene glycol.

28. The method of claim 27, wherein the ink formulation comprises about 10% to about 30% by weight polycaprolactone and about 10% to about 30% by weight polyethylene glycol.

29. The method of claim 24 or claim 25, wherein the three-dimensional structure comprises about 100% by weight ceramic.

30. The method of claim 24 or claim 25, wherein the three-dimensional structure comprises about 100% by weight beta-tricalcium phosphate (β-TCP).

31. The method of claim 24 or claim 25, wherein the three-dimensional structure comprises about 70% to about 80% by weight ceramic, and about 20% to about 30% by weight polymer.

32. The method of claim 24 or claim 25, wherein the three-dimensional structure comprises about 70% to about 80% by weight beta-tricalcium phosphate (β-TCP), and about 20% to about 30% by weight polycaprolactone.

33. The method of claim 24 or claim 25, further comprising combining the three-dimensional structure with a therapeutic agent.

34. The method of claim 33, wherein the therapeutic agent comprises a mammalian growth factor or a functional portion thereof.

35. The method of claim 33, wherein the therapeutic agent comprises one or more polypeptides selected from Table 4, or a functional portion thereof.

36. The method of claim 33, wherein the therapeutic agent comprises a bone morphogenetic protein (BMP).

37. The method of claim 33, wherein the therapeutic agent comprises a targeting moiety that non-covalently binds to the three-dimensional structure.

38. The method of claim 37, wherein the targeting moiety binds to the printed three-dimensional structure with an affinity of about 1 pM to about 100 μm.

39. The method of claim 37, wherein the targeting moiety comprises a polypeptide at least about 70%, 75%, 80%, 85%, 90%, 95%, or 100% identical to any one of the sequences of Tables 5-6.

40. The method of claim 37, wherein the targeting moiety comprises about 2, 3, 4, 5, 6, 7, 8, 9, or 10 sequences selected from the sequences of Tables 5-6.

41. The method of claim 33, wherein the therapeutic agent is a chimeric polypeptide comprising a sequence at least about 70%, 75%, 80%, 85%, 90%, 95%, or 100% identical to any one of SEQ ID NOS: 794-802.

42. A method of treating a condition in a subject in need thereof, the method comprising administering to the subject the three-dimensional structure manufactured by the method of claim 24 or claim 25.

43. The method of claim 42, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.

44. The method of claim 42, wherein the method comprises spinal fusion.

45. The method of claim 44, wherein the spinal fusion comprises posterior lumbar fusion (PLF) and/or interbody fusion.

46. The method of claim 42, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

47. An ink formulation for three-dimensional printing, the formulation comprising about 30% to about 70% by weight ceramic, and about 5% to about 30% by weight polymer.

48. The ink formulation of claim 47, wherein the ceramic comprises calcium phosphate, hydroxyapatite, fluorapatite, bone, silicate, or vanadate, or a combination thereof.

49. The ink formulation of claim 47 or claim 48, wherein the ceramic comprises beta-tricalcium phosphate (β-TCP).

50. The ink formulation of claim 47 or claim 48, comprising about 50% to about 70% by weight ceramic, about 10% to about 30% by weight a first polymer, and about 10% to about 30% by weight a second polymer.

51. The ink formulation of claim 47 or claim 48, comprising about 50% to about 70% by weight beta-tricalcium phosphate (β-TCP), about 10% to about 30% by weight a first polymer comprising polycaprolactone, and about 10% to about 30% by weight a second polymer comprising polyethylene glycol.

52. The ink formulation of claim 47 or claim 48, comprising about 50% to about 70% by weight ceramic, about 5% to about 15% by weight polymer, and optionally an anti-foaming agent and/or a dispersing agent.

53. The ink formulation of claim 47 or claim 48, comprising about 50% to about 70% by weight tricalcium phosphate, about 5% to about 15% by weight poloxamer, and optionally an anti-foaming agent and/or a dispersing agent.

54. The ink formulation of claim 52, comprising about 0.1% to about 1% by weight anti-foaming agent, wherein the anti-foaming agent optionally comprises an alcohol.

55. The ink formulation of claim 52, comprising about 0.1% to about 1% by weight dispersing agent, wherein the dispersing agent optionally comprises ammonium polyacrylate.

56. The ink formulation of claim 47 or claim 48, comprising about 40% to about 60% by weight ceramic, about 5% to about 15% by weight polymer, and about 30% to about 40% by weight solvent.

57. The ink formulation of claim 47 or claim 48, comprising about 40% to about 60% by weight beta-tricalcium phosphate (β-TCP), about 5% to about 15% by weight polycaprolactone, and about 30% to about 40% by weight solvent.

58. The ink formulation of claim 56, wherein the solvent comprises dichloromethane, 2-butoxyethanol, dibutyl phthalate, or chloroform, or a combination thereof.

59. A method of preparing a three-dimensional structure, the method comprising using the formation of claim 47 or claim 48 as an ink in a three-dimensional printing method.

60. A three-dimensional structure prepared using the ink formulation of claim 47 or claim 48.

61. The three-dimensional structure of claim 60, comprising about 50% to about 100% by weight ceramic.

62. The three-dimensional structure of claim 60, comprising about 50% to about 100% by weight tricalcium phosphate.

63. The three-dimensional structure of claim 60, comprising about 50% to about 90% by weight tricalcium phosphate and about 10% to about 50% polymer.

64. The three-dimensional structure of claim 63, wherein the polymer comprises polycaprolactone.

65. The three-dimensional structure of claim 60, wherein the structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

66. A three-dimensional structure comprising about 50% to about 100% by weight ceramic, and about 0% to about 50% polymer.

67. The three-dimensional structure of claim 66, wherein the ceramic comprises calcium phosphate, hydroxyapatite, fluorapatite, bone, silicate, or vanadate, or a combination thereof.

68. The three-dimensional structure of claim 66 or claim 67, wherein the ceramic comprises beta-tricalcium phosphate (β-TCP).

69. The three-dimensional structure of claim 66 or claim 67, comprising about 50% to about 100% by weight ceramic.

70. The three-dimensional structure of claim 66 or claim 67, comprising about 100% by weight ceramic.

71. The three-dimensional structure of claim 66, comprising about 100% by weight tricalcium phosphate.

72. The three-dimensional structure of claim 66 or claim 67, comprising about 50% to about 90% by weight ceramic and about 10% to about 50% polymer.

73. The three-dimensional structure of claim 66 or claim 67, comprising about 50% to about 90% by weight tricalcium phosphate and about 10% to about 50% polymer.

74. The three-dimensional structure of claim 72, wherein the polymer comprises polycaprolactone.

75. The three-dimensional structure of claim 66 or claim 67, wherein the structure comprises one or more of a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

76. The three-dimensional structure of claim 66 or claim 67, prepared by three-dimensional printing methods.

77. A method of delivering a therapeutic agent to a subject in need thereof, the method comprising delivering to an organ or tissue of the subject a device comprising a therapeutic agent and the three-dimensional structure of claim 66 or claim 67.

78. A device comprising a therapeutic agent and the three-dimensional structure of claim 66 or claim 67.

79. The method of claim 77, wherein the therapeutic agent comprises a mammalian growth factor or functional portion thereof.

80. The method of claim 77, wherein the therapeutic agent comprises one or more polypeptides selected from Table 4, or a functional portion thereof.

81. The method of claim 77, wherein the therapeutic agent comprises a bone morphogenetic protein (BMP).

82. The method of claim 77, wherein the device comprises a targeting moiety.

83. The method of claim 82, wherein the targeting moiety comprises a polypeptide comprising one or more sequences at least about 70%, 75%, 80%, 85%, 90%, 95%, or 100% identical to any one of the sequences of Tables 5-6.

84. The method of claim 82, wherein the targeting moiety comprises about 2, 3, 4, 5, 6, 7, 8, 9, or 10 sequences selected from the sequences of Tables 5-6.

85. The method of claim 82, wherein the targeting moiety non-covalently binds to the three-dimensional structure.

86. The method of claim 82, wherein the targeting moiety is connected to the therapeutic agent in a chimeric polypeptide.

87. The method of claim 86, wherein the chimeric polypeptide comprises a sequence at least about 70%, 75%, 80%, 85%, 90%, 95%, or 100% identical to any one of SEQ ID NOS: 794-802.

88. A method of preparing the device of claim 78, the method comprising combining the therapeutic agent and the three-dimensional structure, where the therapeutic agent non-covalently binds to the three-dimensional structure.

89. A method of treating a condition in a subject in need thereof, the method comprising administering to the subject the three-dimensional structure of claim 66 or claim 67.

90. The method of claim 89, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.

91. The method of claim 89, wherein the method comprises spinal fusion.

92. The method of claim 91, wherein the spinal fusion comprises posterior lumbar fusion (PLF) and/or interbody fusion.

93. The method of claim 89, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

94. The device of claim 78, wherein the therapeutic agent comprises a mammalian growth factor or functional portion thereof.

95. The device of claim 78, wherein the therapeutic agent comprises one or more polypeptides selected from Table 4, or a functional portion thereof.

96. The device of claim 78, wherein the therapeutic agent comprises a bone morphogenetic protein (BMP).

97. The device of claim 78, wherein the device comprises a targeting moiety.

98. The device of claim 82, wherein the targeting moiety comprises a polypeptide comprising one or more sequences at least about 70%, 75%, 80%, 85%, 90%, 95%, or 100% identical to any one of the sequences of Tables 5-6.

99. The device of claim 82, wherein the targeting moiety comprises about 2, 3, 4, 5, 6, 7, 8, 9, or 10 sequences selected from the sequences of Tables 5-6.

100. The device of claim 78, wherein the targeting moiety non-covalently binds to the three-dimensional structure.

101. The device of claim 78, wherein the targeting moiety is connected to the therapeutic agent in a chimeric polypeptide.

102. The device of claim 86, wherein the chimeric polypeptide comprises a sequence at least about 70%, 75%, 80%, 85%, 90%, 95%, or 100% identical to any one of SEQ ID NOS: 794-802.

103. A method of treating a condition in a subject in need thereof, the method comprising administering to the subject the device of any one of claim 78.

104. The method of claim 89, wherein the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof.

105. The method of claim 89, wherein the method comprises spinal fusion.

106. The method of claim 91, wherein the spinal fusion comprises posterior lumbar fusion (PLF) and/or interbody fusion.

107. The method of claim 89, wherein the method comprises bone repair, dental repair, craniomaxillofacial repair, ankle fusion, kyphoplasty, osteoplasty, scaphoid fracture repair, tendeno-osseous repair, costal reconstruction, subchondral bone repair, cartilage repair, or surgical implantation of the three-dimensional structure or device, or a combination thereof.

107. The device of claim 1 or claim 2, wherein the three-dimensional structure has a density of between about 1 g/cm³ and about 3 g/cm³.

108. The device of claim 1 or claim 2, wherein the three-dimensional structure has an open porosity of between about 15% and about 45%.

109. The device of claim 1 or claim 2, wherein the three-dimensional structure has a specific surface area of between about 0.50 m²/g and about 1.0 m²/g.

110. The device of claim 1 or claim 2, wherein the three-dimensional structure has a fiber diameter of about 325 μm and about 475 μm.

111. The device claim 1 or claim 2, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

112. The device of claim 1 or claim 2, wherein the three-dimensional structure has a density of about 2.44 g/cm³, open porosity of about 19.6%, and a fiber diameter of about 384 μm.

113. The device of claim 1 or claim 2, wherein the three-dimensional structure has a density of about 1.32 g/cm³, open porosity of about 38%, and a fiber diameter of about 394 μm.

114. The device of claim 1 or claim 2, wherein the three-dimensional structure has a density of about 1.49 g/cm³, open porosity of about 31%, specific surface area of 0.81 m²/g, and a fiber diameter of about 420 μm.

115. The method of claim 19, wherein the three-dimensional structure has a density of between about 1 g/cm³ and about 3 g/cm³.

116. The method of claim 19, wherein the three-dimensional structure has an open porosity of between about 15% and about 45%.

117. The method of claim 19, wherein the three-dimensional structure has a specific surface area of between about 0.50 m²/g and about 1.0 m²/g.

118. The method of claim 19, wherein the three-dimensional structure has a fiber diameter of about 325 μm and about 475 μm.

120. The method of claim 19, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

121. The method of claim 19, wherein the three-dimensional structure has a density of about 2.44 g/cm³, open porosity of about 19.6%, and a fiber diameter of about 384 μm.

122. The method of claim 19, wherein the three-dimensional structure has a density of about 1.32 g/cm³, open porosity of about 38%, and a fiber diameter of about 394 μm.

123. The method of claim 19, wherein the three-dimensional structure has a density of about 1.49 g/cm³, open porosity of about 31%, specific surface area of 0.81 m²/g, and a fiber diameter of about 420 μm.

124. The method of claim 24, wherein the three-dimensional structure has a density of between about 1 g/cm³ and about 3 g/cm³.

125. The method of claim 24, wherein the three-dimensional structure has an open porosity of between about 15% and about 45%.

126. The method of claim 24, wherein the three-dimensional structure has a specific surface area of between about 0.50 m²/g and about 1.0 m²/g.

127. The method of claim 24, wherein the three-dimensional structure has a fiber diameter of about 325 μm and about 475 μm.

128. The method of claim 24, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

129. The method of claim 24, wherein the three-dimensional structure has a density of about 2.44 g/cm³, open porosity of about 19.6%, and a fiber diameter of about 384 μm.

130. The method of claim 24, wherein the three-dimensional structure has a density of about 1.32 g/cm³, open porosity of about 38%, and a fiber diameter of about 394 μm.

131. The method of claim 24, wherein the three-dimensional structure has a density of about 1.49 g/cm³, open porosity of about 31%, specific surface area of 0.81 m²/g, and a fiber diameter of about 420 μm.

132. The method of claim 42, wherein the three-dimensional structure has a density of between about 1 g/cm³ and about 3 g/cm³.

133. The method of claim 42, wherein the three-dimensional structure has an open porosity of between about 15% and about 45%.

134. The method of claim 42, wherein the three-dimensional structure has a specific surface area of between about 0.50 m²/g and about 1.0 m²/g.

135. The method of claim 42, wherein the three-dimensional structure has a fiber diameter of about 325 μm and about 475 μm.

136. The method of claim 42, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

137. The method of claim 42, wherein the three-dimensional structure has a density of about 2.44 g/cm³, open porosity of about 19.6%, and a fiber diameter of about 384 μm.

138. The method of claim 42, wherein the three-dimensional structure has a density of about 1.32 g/cm³, open porosity of about 38%, and a fiber diameter of about 394 μm.

139. The method of claim 42, wherein the three-dimensional structure has a density of about 1.49 g/cm³, open porosity of about 31%, specific surface area of 0.81 m²/g, and a fiber diameter of about 420 μm.

140. The method of claim 77, wherein the three-dimensional structure has a density of between about 1 g/cm³ and about 3 g/cm³.

141. The method of claim 77, wherein the three-dimensional structure has an open porosity of between about 15% and about 45%.

142. The method of claim 77, wherein the three-dimensional structure has a specific surface area of between about 0.50 m²/g and about 1.0 m²/g.

143. The method of claim 77, wherein the three-dimensional structure has a fiber diameter of about 325 μm and about 475 μm.

144. The method of claim 77, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

145. The method of claim 77, wherein the three-dimensional structure has a density of about 2.44 g/cm³, open porosity of about 19.6%, and a fiber diameter of about 384 μm.

146. The method of claim 77, wherein the three-dimensional structure has a density of about 1.32 g/cm³, open porosity of about 38%, and a fiber diameter of about 394 μm.

147. The method of claim 77, wherein the three-dimensional structure has a density of about 1.49 g/cm³, open porosity of about 31%, specific surface area of 0.81 m²/g, and a fiber diameter of about 420 μm.

148. The device of claim 78, wherein the three-dimensional structure has a density of between about 1 g/cm³ and about 3 g/cm³.

194. The device of claim 78, wherein the three-dimensional structure has an open porosity of between about 15% and about 45%.

150. The device of claim 78, wherein the three-dimensional structure has a specific surface area of between about 0.50 m²/g and about 1.0 m²/g.

151. The device of claim 78, wherein the three-dimensional structure has a fiber diameter of about 325 μm and about 475 μm.

152. The device claim 78, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

153. The device of claim 78, wherein the three-dimensional structure has a density of about 2.44 g/cm³, open porosity of about 19.6%, and a fiber diameter of about 384 μm.

154. The device of claim 78, wherein the three-dimensional structure has a density of about 1.32 g/cm³, open porosity of about 38%, and a fiber diameter of about 394 μm.

155. The device of claim 78, wherein the three-dimensional structure has a density of about 1.49 g/cm³, open porosity of about 31%, specific surface area of 0.81 m²/g, and a fiber diameter of about 420 μm.

156. The method of claim 79, wherein the three-dimensional structure has a density of between about 1 g/cm³ and about 3 g/cm³.

157. The method of claim 79, wherein the three-dimensional structure has an open porosity of between about 15% and about 45%.

158. The method of claim 79, wherein the three-dimensional structure has a specific surface area of between about 0.50 m²/g and about 1.0 m²/g.

159. The method of claim 79, wherein the three-dimensional structure has a fiber diameter of about 325 μm and about 475 μm.

160. The method of claim 79, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

161. The method of claim 79, wherein the three-dimensional structure has a density of about 2.44 g/cm³, open porosity of about 19.6%, and a fiber diameter of about 384 μm.

162. The method of claim 79, wherein the three-dimensional structure has a density of about 1.32 g/cm³, open porosity of about 38%, and a fiber diameter of about 394 μm.

163. The method of claim 79, wherein the three-dimensional structure has a density of about 1.49 g/cm³, open porosity of about 31%, specific surface area of 0.81 m²/g, and a fiber diameter of about 420 μm.

164. The three-dimensional structure of claim 60, wherein the three-dimensional structure has a density of between about 1 g/cm³ and about 3 g/cm³.

165. The three-dimensional structure of claim 60, wherein the three-dimensional structure has an open porosity of between about 15% and about 45%.

166. The three-dimensional structure of claim 60, wherein the three-dimensional structure has a specific surface area of between about 0.50 m²/g and about 1.0 m²/g.

167. The three-dimensional structure of claim 60, wherein the three-dimensional structure has a fiber diameter of about 325 μm and about 475 μm.

168. The three-dimensional structure of claim 60, wherein the three-dimensional structure has a density of between about 1 g/cm³ and about 3 g/cm³, an open porosity of between about 15% and about 45%, a specific surface area of between about 0.50 m²/g and about 1.0 m²/g, and a three-dimensional structure has a fiber diameter of about 325 μm and about 475 μm.

169. The three-dimensional structure of claim 60, wherein the three-dimensional structure has a density of about 2.44 g/cm³, open porosity of about 19.6%, and a fiber diameter of about 384 μm.

170. The three-dimensional structure of claim 60, wherein the three-dimensional structure has a density of about 1.32 g/cm³, open porosity of about 38%, and a fiber diameter of about 394 μm.

171. The three-dimensional structure of claim 60, wherein the three-dimensional structure has a density of about 1.49 g/cm³, open porosity of about 31%, specific surface area of 0.81 m²/g, and a fiber diameter of about 420 μm.


SEQUENCE LISTING (501 sequences)

SEQ ID NO: 1 - protein, 12 aa, Artificial Sequence
  Description of Artificial Sequence: Synthetic peptide
LLADTTHHRPWT

SEQ ID NO: 2 - protein, 12 aa, Artificial Sequence
  Description of Artificial Sequence: Synthetic peptide
VIGESTHHRPWS

SEQ ID NO: 3 - protein, 12 aa, Artificial Sequence
  Description of Artificial Sequence: Synthetic peptide
LIADSTHHSPWT

SEQ ID NO: 4 - protein, 12 aa, Artificial Sequence
  Description of Artificial Sequence: Synthetic peptide
ILAESTHHKPWT

SEQ ID NO: 5 - protein, 12 aa, Artificial Sequence
  Description of Artificial Sequence: Synthetic peptide
ILAETTHHRPWS

SEQ ID NO: 6 - protein, 12 aa, Artificial Sequence
  Description of Artificial Sequence: Synthetic peptide
IIGESSHHKPFT

SEQ ID NO: 7 - protein, 12 aa, Artificial Sequence
  Description of Artificial Sequence: Synthetic peptide
GLGDTTHHRPWG

SEQ ID NO: 8 - protein, 12 aa, Artificial Sequence
  Description of Artificial Sequence: Synthetic peptide
VLGDTTHHKPWT

SEQ ID NO: 9 - protein, 12 aa, Artificial Sequence
  Description of Artificial Sequence: Synthetic peptide
IVADSTHHRPWT

SEQ ID NO: 10 - protein, 11 aa, Artificial Sequence
  Description of Artificial Sequence: Synthetic peptide
STADTSHHRPS

SEQ ID NO: 11 - protein, 12 aa, Artificial Sequence
  Description of Artificial Sequence: Synthetic peptide
TSGGESTHHRPS

SEQ ID NO: 12 - protein, 12 aa, Artificial Sequence
  Description of Artificial Sequence: Synthetic peptide
TSGGESSHHKPS

SEQ ID NO: 13 - protein, 12 aa, Artificial Sequence
  Description of Artificial Sequence: Synthetic peptide
TGSGDSSHHRPS

SEQ ID NO: 14 - protein, 13 aa, Artificial Sequence
  Description of Artificial Sequence: Synthetic peptide
GSSGESTHHKPST

SEQ ID NO: 15 - protein, 12 aa, Artificial Sequence
  Description of Artificial Sequence: Synthetic peptide
VGADSTHHRPVT

SEQ ID NO: 16 - protein, 12 aa, Artificial Sequence
  Description of Artificial Sequence: Synthetic peptide
GAADTTHHRPVT

SEQ ID NO: 17 - protein, 12 aa, Artificial Sequence
  Description of Artificial Sequence: Synthetic peptide
AGADTTHHRPVT

SEQ ID NO: 18 - protein, 12 aa, Artificial Sequence
  Description of Artificial Sequence: Synthetic peptide
GGADTTHHRPAT

SEQ ID NO: 19 - protein, 12 aa, Artificial Sequence
  Description of Artificial Sequence: Synthetic peptide
GGADTTHHRPGT

SEQ ID NO: 20 - protein, 24 aa, Artificial Sequence
  Description of Artificial Sequence: Synthetic peptide
LLADTTHHRPWTVIGESTHHRPWS

SEQ ID NO: 21 - protein, 36 aa, Artificial Sequence
  Description of Artificial Sequence: Synthetic polypeptide
LLADTTHHRPWTVIGESTHHRPWSIIGESSHHKPFT

SEQ ID NO: 22 - protein, 60 aa, Artificial Sequence
  Description of Artificial Sequence: Synthetic polypeptide
LLADTTHHRPWTVIGESTHHRPWSIIGESSHHKPFTGLGDTTHHRPWGILAESTHHKPWT

SEQ ID NO: 23 - protein, 24 aa, Artificial Sequence
  Description of Artificial Sequence: Synthetic peptide
LLADTTHHRPWTILAESTHHKPWT

SEQ ID NO: 24 - protein, 60 aa, Artificial Sequence
  Description of Artificial Sequence: Synthetic polypeptide
LLADTTHHRPWTILAESTHHKPWTLLADTTHHRPWTILAESTHHKPWTLLADTTHHRPWT

SEQ ID NO: 25 - protein, 24 aa, Artificial Sequence
  Description of Artificial Sequence: Synthetic peptide
LLADTTHHRPWTGLGDTTHHRPWG

SEQ ID NO: 26 - protein, 36 aa, Artificial Sequence
  Description of Artificial Sequence: Synthetic polypeptide
LLADTTHHRPWTGLGDTTHHRPWGLLADTTHHRPWT

SEQ ID NO: 27 - protein, 60 aa, Artificial Sequence
  Description of Artificial Sequence: Synthetic polypeptide
LLADTTHHRPWTGLGDTTHHRPWGLLADTTHHRPWTGLGDTTHHRPWGLLADTTHHRPWT

SEQ ID NO: 28 - protein, 84 aa, Artificial Sequence
  Description of Artificial Sequence: Synthetic polypeptide
LLADTTHHRPWTGLGDTTHHRPWGLLADTTHHRPWTGLGDTTHHRPWGLLADTTHHRPWTGLGDTTHHRPWGLLADTTHHRPWT

SEQ ID NO: 29 - protein, 60 aa, Artificial Sequence
  Description of Artificial Sequence: Synthetic polypeptide
STADTSHHRPSTSGGESTHHRPSTSGGESSHHKPSTGSGDSSHHRPSGSSGESTHHKPST

SEQ ID NO: 30 - protein, 60 aa, Artificial Sequence
  Description of Artificial Sequence: Synthetic polypeptide
VGADSTHHRPVTGAADTTHHRPVTAGADTTHHRPVTGGADTTHHRPATGGADTTHHRPGT

SEQ ID NO: 31 - protein, 108 aa, Artificial Sequence
  Description of Artificial Sequence: Synthetic polypeptide
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPAT

SEQ ID NO: 32 - protein, 12 aa, Artificial Sequence
  Description of Artificial Sequence: Synthetic peptide
AAADTTHHRPWT

SEQ ID NO: 33 - protein, 60 aa, Artificial Sequence
  Description of Artificial Sequence: Synthetic polypeptide
AAADTTHHRPWTAAADTTHHRPWTAAADTTHHRPWTAAADTTHHRPWTAAADTTHHRPWT

SEQ ID NO: 34 - protein, 60 aa, Artificial Sequence
  Description of Artificial Sequence: Synthetic polypeptide
LLADAAHHRPWTLLADAAHHRPWTLLADAAHHRPWTLLADAAHHRPWTLLADAAHHRPWT

SEQ ID NO: 35 - protein, 60 aa, Artificial Sequence
  Description of Artificial Sequence: Synthetic polypeptide
LLADTTAARPWTLLADTTAARPWTLLADTTAARPWTLLADTTAARPWTLLADTTAARPWT

SEQ ID NO: 36 - protein, 24 aa, Artificial Sequence
  Description of Artificial Sequence: Synthetic peptide
LLADTTHHRPWTLLADTTHHRPWT

SEQ ID NO: 37 - protein, 36 aa, Artificial Sequence
  Description of Artificial Sequence: Synthetic polypeptide
LLADTTHHRPWTLLADTTHHRPWTLLADTTHHRPWT

SEQ ID NO: 38 - protein, 60 aa, Artificial Sequence
  Description of Artificial Sequence: Synthetic polypeptide
LLADTTHHRPWTLLADTTHHRPWTLLADTTHHRPWTLLADTTHHRPWTLLADTTHHRPWT

SEQ ID NO: 39 - protein, 120 aa, Artificial Sequence
  Description of Artificial Sequence: Synthetic polypeptide
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGS

SEQ ID NO: 40 - protein, 48 aa, Artificial Sequence
  Description of Artificial Sequence: Synthetic polypeptide
LLADTTHHRPWTVIGESTHHRPWSIIGESSHHKPFTGLGDTTHHRPWG

SEQ ID NO: 41 - protein, 48 aa, Artificial Sequence
  Description of Artificial Sequence: Synthetic polypeptide
VIGESTHHRPWSIIGESSHHKPFTGLGDTTHHRPWGILAESTHHKPWT

SEQ ID NO: 42 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHK PWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLPT TTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
LLADTTHHRPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 43 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHK PWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLPT TTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
VIGESTHHRPWSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 44 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHK PWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLPT TTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
ILAESTHHKPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 45 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHK PWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLPT TTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
IIGESSHHKPFTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 46 - protein, 132 aa, Artificial Sequence
  Description of Artificial Sequence: Synthetic polypeptide
  MOD_RES (13)..(132): This region may encompass one of the following sequences: “LLADTTHHRPWT” or “VIGESTHHRPWS” or “LIADSTHHSPWT” or “ILAESTHHKPWT” or “ILAETTHHRPWS” or “IIGESSHHKPFT” or “GLGDTTHHRPWG” or “VLGDTTHHKPWT” or “IVADSTHHRPWT” or
  MOD_RES (13)..(132): CONT. FROM ABOVE: “STADTSHHRPS” or “TSGGESTHHRPS” or “TSGGESSHHKPS” or “TGSGDSSHHRPS” or “GSSGESTHHKPST” or “VGADSTHHRPVT” or “GAADTTHHRPVT” or “AGADTTHHRPVT” or “GGADTTHHRPAT” or “GGADTTHHRPGT” or
  MOD_RES (13)..(132): CONT. FROM ABOVE: “LLADTTHHRPWTVIGESTHHRPWS” or “LLADTTHHRPWTVIGESTHHRPWSIIGESSHHKPFT” or “LLADTTHHRPWTVIGESTHHRPWSIIGESSHHKPFTGLGDTTHHRPWGILAESTHHK PWT” or “LLADTTHHRPWTILAESTHHKPWT” or “LLADTTHHRPWTILAESTHHK
  MOD_RES (13)..(132): CONT. FROM ABOVE: PWTLLADTTHHRPWTILAESTHHKPWTLLADTTHHRPWT” or “LLADTTHHRPWTGLGDTTHHRPWG” or “LLADTTHHRPWTGLGDTTHHRPWGLLADTTHHRPWT” or “LLADTTHHRPWTGLGDTTHHRPWGLLADTTHHRPWTGLGDTTHHRPWGLLADTTHHRPWT” or “LLADTTHHRPWTGLGDTTHHRPWGLLADTTHHRPWTGLGD
  MOD_RES (13)..(132): CONT. FROM ABOVE: TTHHRPWGLLADTTHHRPWTGLGDTTHHRPWGLLADTTHHRPWT” or “STADTSHHRPSTSGGESTHHRPSTSGGESSHHKPSTGSGDSSHHRPSGSSGESTHHKPST” or “VGADSTHHRPVTGAADTTHHRPVTAGADTTHHRPVTGGADTTHHRPATGGADTTHHRPGT” or “STADTSHHRPSLLADTTHHRPWTTSGGESTHHRPSVGADSTHHRPVTTSGGESSHHKPSG
  MOD_RES (13)..(132): CONT. FROM ABOVE: AADTTHHRPVTTGSGDSSHHRPSGSSGESTHHKPSTGGADTTHHRPAT” or “AAADTTHHRPWT” or “AAADTTHHRPWTAAADTTHHRPWTAAADTTHHRPWTAAADTTHHRPWTAAADTTHHRPWT” or “LLADAAHHRPWTLLADAAHHRPWTLLADAAHHRPWTLLADAAHHRPWTLLADAAHHRPWT” or “LLADTTAARPWTLLADTTAARPWTLLADTT
  MOD_RES (13)..(132): CONT. FROM ABOVE: AARPWTLLADTTAARPWTLLADTTAARPWT” or “LLADTTHHRPWTLLADTTHHRPWT” or “LLADTTHHRPWTLLADTTHHRPWTLLADTTHHRPWT” or “LLADTTHHRPWTLLADTTHHRPWTLLADTTHHRPWTLLADTTHHRPWTLLADTTHHRPWT” or “STSGSTVIGESTHHRPWSLIADSTHHSPWTILAESTHHKPWT
  MOD_RES (13)..(132): CONT. FROM ABOVE: ILAETTHHRPWSIIGESSHHKPFTGLGDTTHHRPWGVLGDTTHHKPWTIVADSTHHRPWTGQVLPT TTPSSPSTTSGS” or “LLADTTHHRPWTVIGESTHHRPWSIIGESSHHKPFTGLGDTTHHRPWG” or “VIGESTHHRPWSIIGESSHHKPFTGLGDTTHHRPWGILAESTHHKPWT”
  See specification as filed for detailed description of substitutions and preferred embodiments
GLGDTTHHRPWGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 47 - protein, 24 aa, Artificial Sequence
  Description of Artificial Sequence: Synthetic peptide
  MOD_RES (13)..(24): This region may encompass one of the following sequences: “VIGESTHHRPWS” or “ILAESTHHKPWT” or “IIGESSHHKPFT” or “GLGDTTHHRPWG”
  See specification as filed for detailed description of substitutions and preferred embodiments
LLADTTHHRPWTXXXXXXXXXXXX

SEQ ID NO: 48 - protein, 24 aa, Artificial Sequence
  Description of Artificial Sequence: Synthetic peptide
  MOD_RES (13)..(24): This region may encompass one of the following sequences: “LLADTTHHRPWT” or “ILAESTHHKPWT” or “IIGESSHHKPFT” or “GLGDTTHHRPWG”
  See specification as filed for detailed description of substitutions and preferred embodiments
VIGESTHHRPWSXXXXXXXXXXXX

SEQ ID NO: 49 - protein, 24 aa, Artificial Sequence
  Description of Artificial Sequence: Synthetic peptide
  MOD_RES (13)..(24): This region may encompass one of the following sequences: “LLADTTHHRPWT” or “VIGESTHHRPWS” or “IIGESSHHKPFT” or “GLGDTTHHRPWG”
  See specification as filed for detailed description of substitutions and preferred embodiments
ILAESTHHKPWTXXXXXXXXXXXX

SEQ ID NO: 50 - protein, 24 aa, Artificial Sequence
  Description of Artificial Sequence: Synthetic peptide
  MOD_RES (13)..(24): This region may encompass one of the following sequences: “LLADTTHHRPWT” or “ILAESTHHKPWT” or “VIGESTHHRPWS” or “GLGDTTHHRPWG”
  See specification as filed for detailed description of substitutions and preferred embodiments
IIGESSHHKPFTXXXXXXXXXXXX

SEQ ID NO: 51 - protein, 24 aa, Artificial Sequence
  Description of Artificial Sequence: Synthetic peptide
  MOD_RES (13)..(24): This region may encompass one of the following sequences: “LLADTTHHRPWT” or “ILAESTHHKPWT” or “IIGESSHHKPFT” or “VIGESTHHRPWS”
  See specification as filed for detailed description of substitutions and preferred embodiments
GLGDTTHHRPWGXXXXXXXXXXXX

SEQ ID NO: 52 - protein, 36 aa, Artificial Sequence
  Description of Artificial Sequence: Synthetic polypeptide
  MOD_RES (13)..(36): This region may encompass two of the following sequences: “VIGESTHHRPWS” or “ILAESTHHKPWT” or “IIGESSHHKPFT” or “GLGDTTHHRPWG”
  See specification as filed for detailed description of substitutions and preferred embodiments
LLADTTHHRPWTXXXXXXXXXXXXXXXXXXXXXXXX

SEQ ID NO: 53 - protein, 36 aa, Artificial Sequence
  Description of Artificial Sequence: Synthetic polypeptide
  MOD_RES (13)..(36): This region may encompass two of the following sequences: “LLADTTHHRPWT” or “ILAESTHHKPWT” or “IIGESSHHKPFT” or “GLGDTTHHRPWG”
  See specification as filed for detailed description of substitutions and preferred embodiments
VIGESTHHRPWSXXXXXXXXXXXXXXXXXXXXXXXX

SEQ ID NO: 54 - protein, 36 aa, Artificial Sequence
  Description of Artificial Sequence: Synthetic polypeptide
  MOD_RES (13)..(36): This region may encompass two of the following sequences: “LLADTTHHRPWT” or “VIGESTHHRPWS” or “IIGESSHHKPFT” or “GLGDTTHHRPWG”
  See specification as filed for detailed description of substitutions and preferred embodiments
ILAESTHHKPWTXXXXXXXXXXXXXXXXXXXXXXXX

SEQ ID NO: 55 - protein, 36 aa, Artificial Sequence
  Description of Artificial Sequence: Synthetic polypeptide
  MOD_RES (13)..(36): This region may encompass two of the following sequences: “LLADTTHHRPWT” or “ILAESTHHKPWT” or “VIGESTHHRPWS” or “GLGDTTHHRPWG”
  See specification as filed for detailed description of substitutions and preferred embodiments
IIGESSHHKPFTXXXXXXXXXXXXXXXXXXXXXXXX

SEQ ID NO: 56 - protein, 36 aa, Artificial Sequence
  Description of Artificial Sequence: Synthetic polypeptide
  MOD_RES (13)..(36): This region may encompass two of the following sequences: “LLADTTHHRPWT” or “ILAESTHHKPWT” or “IIGESSHHKPFT” or “VIGESTHHRPWS”
  See specification as filed for detailed description of substitutions and preferred embodiments
GLGDTTHHRPWGXXXXXXXXXXXXXXXXXXXXXXXX

SEQ ID NO: 57 - protein, 48 aa, Artificial Sequence
  Description of Artificial Sequence: Synthetic polypeptide
  MOD_RES (13)..(48): This region may encompass three of the following sequences: “VIGESTHHRPWS” or “ILAESTHHKPWT” or “IIGESSHHKPFT” or “GLGDTTHHRPWG”
  See specification as filed for detailed description of substitutions and preferred embodiments
LLADTTHHRPWTXXXXXXXXXXXXXXXXXXXXXXXXXXXXXXXXXXXX

SEQ ID NO: 58 - protein, 48 aa, Artificial Sequence
  Description of Artificial Sequence: Synthetic polypeptide
  MOD_RES (13)..(48): This region may encompass three of the following sequences: “LLADTTHHRPWT” or “ILAESTHHKPWT” or “IIGESSHHKPFT” or “GLGDTTHHRPWG”
  See specification as filed for detailed description of substitutions and preferred embodiments
VIGESTHHRPWSXXXXXXXXXXXXXXXXXXXXXXXXXXXXXXXXXXXX

SEQ ID NO: 59 - protein, 48 aa, Artificial Sequence
  Description of Artificial Sequence: Synthetic polypeptide
  MOD_RES (13)..(48): This region may encompass three of the following sequences: “LLADTTHHRPWT” or “VIGESTHHRPWS” or “IIGESSHHKPFT” or “GLGDTTHHRPWG”
  See specification as filed for detailed description of substitutions and preferred embodiments
ILAESTHHKPWTXXXXXXXXXXXXXXXXXXXXXXXXXXXXXXXXXXXX

SEQ ID NO: 60 - protein, 48 aa, Artificial Sequence
  Description of Artificial Sequence: Synthetic polypeptide
  MOD_RES (13)..(48): This region may encompass three of the following sequences: “LLADTTHHRPWT” or “ILAESTHHKPWT” or “VIGESTHHRPWS” or “GLGDTTHHRPWG”
  See specification as filed for detailed description of substitutions and preferred embodiments
IIGESSHHKPFTXXXXXXXXXXXXXXXXXXXXXXXXXXXXXXXXXXXX

SEQ ID NO: 61 - protein, 48 aa, Artificial Sequence
  Description of Artificial Sequence: Synthetic polypeptide
  MOD_RES (13)..(48): This region may encompass three of the following sequences: “LLADTTHHRPWT” or “ILAESTHHKPWT” or “IIGESSHHKPFT” or “VIGESTHHRPWS”
  See specification as filed for detailed description of substitutions and preferred embodiments
GLGDTTHHRPWGXXXXXXXXXXXXXXXXXXXXXXXXXXXXXXXXXXXX

SEQ ID NO: 62 - protein, 10 aa, Artificial Sequence
  Description of Artificial Sequence: Synthetic peptide
ACAPLMFSQC

SEQ ID NO: 63 - protein, 10 aa, Artificial Sequence
  Description of Artificial Sequence: Synthetic peptide
ACHASLKHRC

SEQ ID NO: 64 - protein, 10 aa, Artificial Sequence
  Description of Artificial Sequence: Synthetic peptide
ACLSTKTNIC

SEQ ID NO: 65 - protein, 10 aa, Artificial Sequence
  Description of Artificial Sequence: Synthetic peptide
ACTTPSKHQC

SEQ ID NO: 66 - protein, 11 aa, Artificial Sequence
  Description of Artificial Sequence: Synthetic peptide
AHFSPNLLLGG

SEQ ID NO: 67 - protein, 12 aa, Artificial Sequence
  Description of Artificial Sequence: Synthetic peptide
AHSLKSITNHGL

SEQ ID NO: 68 - protein, 7 aa, Artificial Sequence
  Description of Artificial Sequence: Synthetic peptide
AKQTVPV

SEQ ID NO: 69 - protein, 12 aa, Artificial Sequence
  Description of Artificial Sequence: Synthetic peptide
AKTLMPSPFPRT

SEQ ID NO: 70 - protein, 12 aa, Artificial Sequence
  Description of Artificial Sequence: Synthetic peptide
AMSQTMTAAIEK

SEQ ID NO: 71 - protein, 7 aa, Artificial Sequence
  Description of Artificial Sequence: Synthetic peptide
ANPPLSL

SEQ ID NO: 72 - protein, 7 aa, Artificial Sequence
  Description of Artificial Sequence: Synthetic peptide
ANPYHRH

SEQ ID NO: 73 - protein, 7 aa, Artificial Sequence
  Description of Artificial Sequence: Synthetic peptide
APLSLSL

SEQ ID NO: 74 - protein, 16 aa, Artificial Sequence
  Description of Artificial Sequence: Synthetic peptide
APYHPTIPASVHGGGK

SEQ ID NO: 75 - protein, 12 aa, Artificial Sequence
  Description of Artificial Sequence: Synthetic peptide
  MOD_RES (12)..(12): Any amino acid
ASAVGSLSIRWX

SEQ ID NO: 76 - protein, 7 aa, Artificial Sequence
  Description of Artificial Sequence: Synthetic peptide
ASGPTNV

SEQ ID NO: 77 - protein, 7 aa, Artificial Sequence
  Description of Artificial Sequence: Synthetic peptide
ASHNPKL

SEQ ID NO: 78 - protein, 12 aa, Artificial Sequence
  Description of Artificial Sequence: Synthetic peptide
ASWVDSRQPSAA

SEQ ID NO: 79 - protein, 7 aa, Artificial Sequence
  Description of Artificial Sequence: Synthetic peptide
ATFSPPL

SEQ ID NO: 80 - protein, 12 aa, Artificial Sequence
  Description of Artificial Sequence: Synthetic peptide
ATWSHHLSSAGL

SEQ ID NO: 81 - protein, 16 aa, Artificial Sequence
  Description of Artificial Sequence: Synthetic peptide
ATWSHHLSSAGLGGGS

SEQ ID NO: 82 - protein, 9 aa, Artificial Sequence
  Description of Artificial Sequence: Synthetic peptide
CAHLSPHKC

SEQ ID NO: 83 - protein, 9 aa, Artificial Sequence
  Description of Artificial Sequence: Synthetic peptide
CDIPWRNEC

SEQ ID NO: 84 - protein, 9 aa, Artificial Sequence
  Description of Artificial Sequence: Synthetic peptide
CDPLRQHSC

SEQ ID NO: 85 - protein, 9 aa, Artificial Sequence
  Description of Artificial Sequence: Synthetic peptide
CDSLGHWLC

SEQ ID NO: 86 - protein, 9 aa, Artificial Sequence
  Description of Artificial Sequence: Synthetic peptide
CDYTTRHSC

SEQ ID NO: 87 - protein, 9 aa, Artificial Sequence
  Description of Artificial Sequence: Synthetic peptide
CHGTLNPEC

SEQ ID NO: 88 - protein, 9 aa, Artificial Sequence
  Description of Artificial Sequence: Synthetic peptide
CHHNLSWEC

SEQ ID NO: 89 - protein, 9 aa, Artificial Sequence
  Description of Artificial Sequence: Synthetic peptide
CHIWTLASC

SEQ ID NO: 90 - protein, 9 aa, Artificial Sequence
  Description of Artificial Sequence: Synthetic peptide
CHNTFSPRC

SEQ ID NO: 91 - protein, 9 aa, Artificial Sequence
  Description of Artificial Sequence: Synthetic peptide
CIPLHASLC

SEQ ID NO: 92 - protein, 9 aa, Artificial Sequence
  Description of Artificial Sequence: Synthetic peptide
CITTTSLSC

SEQ ID NO: 93 - protein, 9 aa, Artificial Sequence
  Description of Artificial Sequence: Synthetic peptide
CKLTTCKDC

SEQ ID NO: 94 - protein, 9 aa, Artificial Sequence
  Description of Artificial Sequence: Synthetic peptide
CKNHTTFWC

SEQ ID NO: 95 - protein, 9 aa, Artificial Sequence
  Description of Artificial Sequence: Synthetic peptide
CLKLLSRSC

SEQ ID NO: 96 - protein, 9 aa, Artificial Sequence
  Description of Artificial Sequence: Synthetic peptide
CLLKAHPSC

SEQ ID NO: 97 - protein, 9 aa, Artificial Sequence
  Description of Artificial Sequence: Synthetic peptide
CLNQLKQAC

SEQ ID NO: 98 - protein, 9 aa, Artificial Sequence
  Description of Artificial Sequence: Synthetic peptide
CLSTKTNIC

SEQ ID NO: 99 - protein, 9 aa, Artificial Sequence
  Description of Artificial Sequence: Synthetic peptide
CMNFPSPHC

SEQ ID NO: 100 - protein, 9 aa, Artificial Sequence
  Description of Artificial Sequence: Synthetic peptide
CNYPTLKSC

SEQ ID NO: 101 - protein, 11 aa, Artificial Sequence
  Description of Artificial Sequence: Synthetic peptide
CPQLTVGQHRT

SEQ ID NO: 102 - protein, 9 aa, Artificial Sequence
  Description of Artificial Sequence: Synthetic peptide
CPQSPTYTC

SEQ ID NO: 103 - protein, 9 aa, Artificial Sequence
  Description of Artificial Sequence: Synthetic peptide
CPSSAIHTC

SEQ ID NO: 104 - protein, 9 aa, Artificial Sequence
  Description of Artificial Sequence: Synthetic peptide
CPTSTARIC

SEQ ID NO: 105 - protein, 9 aa, Artificial Sequence
  Description of Artificial Sequence: Synthetic peptide
CQASSFPSC

SEQ ID NO: 106 - protein, 9 aa, Artificial Sequence
  Description of Artificial Sequence: Synthetic peptide
CQPYFWYRC

SEQ ID NO: 107 - protein, 9 aa, Artificial Sequence
  Description of Artificial Sequence: Synthetic peptide
CQTLTPSIC

SEQ ID NO: 108 - protein, 9 aa, Artificial Sequence
  Description of Artificial Sequence: Synthetic peptide
CSKLGHLWC

SEQ ID NO: 109 - protein, 9 aa, Artificial Sequence
  Description of Artificial Sequence: Synthetic peptide
  MOD_RES (9)..(9): Any amino acid
CSKTPERIX

SEQ ID NO: 110 - protein, 9 aa, Artificial Sequence
  Description of Artificial Sequence: Synthetic peptide
CSNNNRMTC

SEQ ID NO: 111 - protein, 9 aa, Artificial Sequence
  Description of Artificial Sequence: Synthetic peptide
CSPILSLSC

SEQ ID NO: 112 - protein, 9 aa, Artificial Sequence
  Description of Artificial Sequence: Synthetic peptide
CSPTNFTRC

SEQ ID NO: 113 - protein, 9 aa, Artificial Sequence
  Description of Artificial Sequence: Synthetic peptide
CSRPAMNVC

SEQ ID NO: 114 - protein, 9 aa, Artificial Sequence
  Description of Artificial Sequence: Synthetic peptide
CSTKAYPNC

SEQ ID NO: 115 - protein, 9 aa, Artificial Sequence
  Description of Artificial Sequence: Synthetic peptide
CSTSSCGSC

SEQ ID NO: 116 - protein, 9 aa, Artificial Sequence
  Description of Artificial Sequence: Synthetic peptide
CSYWGHRDC

SEQ ID NO: 117 - protein, 9 aa, Artificial Sequence
  Description of Artificial Sequence: Synthetic peptide
CTAHDANAC

SEQ ID NO: 118 - protein, 9 aa, Artificial Sequence
  Description of Artificial Sequence: Synthetic peptide
CTANSEKTC

SEQ ID NO: 119 - protein, 9 aa, Artificial Sequence
  Description of Artificial Sequence: Synthetic peptide
CTHPKASMC

SEQ ID NO: 120 - protein, 9 aa, Artificial Sequence
  Description of Artificial Sequence: Synthetic peptide
CTKTINGKC

SEQ ID NO: 121 - protein, 9 aa, Artificial Sequence
  Description of Artificial Sequence: Synthetic peptide
CTNMQSPLC

SEQ ID NO: 122 - protein, 9 aa, Artificial Sequence
  Description of Artificial Sequence: Synthetic peptide
CTPFTKLPC

SEQ ID NO: 123 - protein, 9 aa, Artificial Sequence
  Description of Artificial Sequence: Synthetic peptide
CTPTTDSIC

SEQ ID NO: 124 - protein, 9 aa, Artificial Sequence
  Description of Artificial Sequence: Synthetic peptide
CTQQNGHPC

SEQ ID NO: 125 - protein, 9 aa, Artificial Sequence
  Description of Artificial Sequence: Synthetic peptide
CTTPSKHQC

SEQ ID NO: 126 - protein, 9 aa, Artificial Sequence
  Description of Artificial Sequence: Synthetic peptide
CTYNVAKPC

SEQ ID NO: 127 - protein, 7 aa, Artificial Sequence
  Description of Artificial Sequence: Synthetic peptide
DKLHRLA

SEQ ID NO: 128 - protein, 12 aa, Artificial Sequence
  Description of Artificial Sequence: Synthetic peptide
DLNYFTLSSKRE

SEQ ID NO: 129 - protein, 12 aa, Artificial Sequence
  Description of Artificial Sequence: Synthetic peptide
DLPPTLHTTGSP

SEQ ID NO: 130 - protein, 7 aa, Artificial Sequence
  Description of Artificial Sequence: Synthetic peptide
DMRQQRS

SEQ ID NO: 131 - protein, 7 aa, Artificial Sequence
  Description of Artificial Sequence: Synthetic peptide
DQYWGLR

SEQ ID NO: 132 - protein, 12 aa, Artificial Sequence
  Description of Artificial Sequence: Synthetic peptide
DSSNPIFWRPSS

SEQ ID NO: 133 - protein, 16 aa, Artificial Sequence
  Description of Artificial Sequence: Synthetic peptide
DSSNPIFWRPSSGGGS

SEQ ID NO: 134 - protein, 12 aa, Artificial Sequence
  Description of Artificial Sequence: Synthetic peptide
EFLGVPASLVNP

SEQ ID NO: 135 - protein, 7 aa, Artificial Sequence
  Description of Artificial Sequence: Synthetic peptide
EPNHTRF

SEQ ID NO: 136 - protein, 9 aa, Artificial Sequence
  Description of Artificial Sequence: Synthetic peptide
EPRRAVAAL

SEQ ID NO: 137 - protein, 9 aa, Artificial Sequence
  Description of Artificial Sequence: Synthetic peptide
EPRRAVAEL

SEQ ID NO: 138 - protein, 9 aa, Artificial Sequence
  Description of Artificial Sequence: Synthetic peptide
EPRREVAEL

SEQ ID NO: 139 - protein, 9 aa, Artificial Sequence
  Description of Artificial Sequence: Synthetic peptide
EPRREVCEL

SEQ ID NO: 140 - protein, 12 aa, Artificial Sequence
  Description of Artificial Sequence: Synthetic peptide
ESDLTHALHWLG

SEQ ID NO: 141 - protein, 7 aa, Artificial Sequence
  Description of Artificial Sequence: Synthetic peptide
ESLKSIS

SEQ ID NO: 142 - protein, 7 aa, Artificial Sequence
  Description of Artificial Sequence: Synthetic peptide
ETRTQLL

SEQ ID NO: 143 - protein, 7 aa, Artificial Sequence
  Description of Artificial Sequence: Synthetic peptide
ETVCASS

SEQ ID NO: 144 - protein, 7 aa, Artificial Sequence
  Description of Artificial Sequence: Synthetic peptide
ETYARPL

SEQ ID NO: 145 - protein, 7 aa, Artificial Sequence
  Description of Artificial Sequence: Synthetic peptide
ETYQQPL

SEQ ID NO: 146 - protein, 12 aa, Artificial Sequence
  Description of Artificial Sequence: Synthetic peptide
EVHSTDRYRSIP

SEQ ID NO: 147 - protein, 12 aa, Artificial Sequence
  Description of Artificial Sequence: Synthetic peptide
FGLQPTGDIARR

SEQ ID NO: 148 - protein, 12 aa, Artificial Sequence
  Description of Artificial Sequence: Synthetic peptide
FSMDDPERVRSP

SEQ ID NO: 149 - protein, 12 aa, Artificial Sequence
  Description of Artificial Sequence: Synthetic peptide
FSPLHTSTYRPS

SEQ ID NO: 150 - protein, 7 aa, Artificial Sequence
  Description of Artificial Sequence: Synthetic peptide
FTLPTIR

SEQ ID NO: 151 - protein, 7 aa, Artificial Sequence
  Description of Artificial Sequence: Synthetic peptide
FVNLLGQ

SEQ ID NO: 152 - protein, 12 aa, Artificial Sequence
  Description of Artificial Sequence: Synthetic peptide
GDFNSGHHTTTR

SEQ ID NO: 153 - protein, 7 aa, Artificial Sequence
  Description of Artificial Sequence: Synthetic peptide
GGGAAAA

SEQ ID NO: 154 - protein, 12 aa, Artificial Sequence
  Description of Artificial Sequence: Synthetic peptide
GIHVPWMPPVAF

SEQ ID NO: 155 - protein, 16 aa, Artificial Sequence
  Description of Artificial Sequence: Synthetic peptide
GIHVPWMPPVAFGGGS

SEQ ID NO: 156 - protein, 12 aa, Artificial Sequence
  Description of Artificial Sequence: Synthetic peptide
GPSNNLPWSNTP

SEQ ID NO: 157 - protein, 12 aa, Artificial Sequence
  Description of Artificial Sequence: Synthetic peptide
GSAGLKYPLYKS

SEQ ID NO: 158 - protein, 7 aa, Artificial Sequence
  Description of Artificial Sequence: Synthetic peptide
GSCPPKK

SEQ ID NO: 159 - protein, 7 aa, Artificial Sequence
  Description of Artificial Sequence: Synthetic peptide
GSLFKAL

SEQ ID NO: 160 - protein, 7 aa, Artificial Sequence
  Description of Artificial Sequence: Synthetic peptide
GTQTPQP

SEQ ID NO: 161 - protein, 7 aa, Artificial Sequence
  Description of Artificial Sequence: Synthetic peptide
GTSRLFS

SEQ ID NO: 162 - protein, 12 aa, Artificial Sequence
  Description of Artificial Sequence: Synthetic peptide
GVHKHFYSRWLG

SEQ ID NO: 163 - protein, 12 aa, Artificial Sequence
  Description of Artificial Sequence: Synthetic peptide
HAPLTRSPAPNL

SEQ ID NO: 164 - protein, 7 aa, Artificial Sequence
  Description of Artificial Sequence: Synthetic peptide
HAPVQPN

SEQ ID NO: 165 - protein, 12 aa, Artificial Sequence
  Description of Artificial Sequence: Synthetic peptide
  MOD_RES (8)..(8): Any amino acid
HGSLTTLXRYEP

SEQ ID NO: 166 - protein, 12 aa, Artificial Sequence
  Description of Artificial Sequence: Synthetic peptide
HHFHLPKLRPPV

SEQ ID NO: 167 - protein, 7 aa, Artificial Sequence
  Description of Artificial Sequence: Synthetic peptide
HHQRSPA

SEQ ID NO: 168 - protein, 12 aa, Artificial Sequence
  Description of Artificial Sequence: Synthetic peptide
HHTWDTRIWQAF

SEQ ID NO: 169 - protein, 7 aa, Artificial Sequence
  Description of Artificial Sequence: Synthetic peptide
HMLAQTF

SEQ ID NO: 170 - protein, 12 aa, Artificial Sequence
  Description of Artificial Sequence: Synthetic peptide
HNVTTRTQRLMP

SEQ ID NO: 171 - protein, 11 aa, Artificial Sequence
  Description of Artificial Sequence: Synthetic peptide
HPTTPIHMPNF

SEQ ID NO: 172 - protein, 12 aa, Artificial Sequence
  Description of Artificial Sequence: Synthetic peptide
HQFISPEPFLIS

SEQ ID NO: 173 - protein, 12 aa, Artificial Sequence
  Description of Artificial Sequence: Synthetic peptide
  MOD_RES (5)..(5): Any amino acid
HQFPXSNLVWKP

SEQ ID NO: 174 - protein, 7 aa, Artificial Sequence
  Description of Artificial Sequence: Synthetic peptide
HQWDHKY

SEQ ID NO: 175 - protein, 13 aa, Artificial Sequence
  Description of Artificial Sequence: Synthetic peptide
  MOD_RES (5)..(5): Any amino acid
  MOD_RES (7)..(7): Any amino acid
  MOD_RES (11)..(11): Any amino acid
HRDPXSXPSAXRP

SEQ ID NO: 176 - protein, 7 aa, Artificial Sequence
  Description of Artificial Sequence: Synthetic peptide
HRLGHMS

SEQ ID NO: 177 - protein, 12 aa, Artificial Sequence
  Description of Artificial Sequence: Synthetic peptide
HSACHASLKHRC

SEQ ID NO: 178 - protein, 12 aa, Artificial Sequence
  Description of Artificial Sequence: Synthetic peptide
HSACKLTTCKDG

SEQ ID NO: 179 - protein, 12 aa, Artificial Sequence
  Description of Artificial Sequence: Synthetic peptide
HSACLSTKTNIC

SEQ ID NO: 180 - protein, 12 aa, Artificial Sequence
  Description of Artificial Sequence: Synthetic peptide
HSMPHMGTYLLT

SEQ ID NO: 181 - protein, 7 aa, Artificial Sequence
  Description of Artificial Sequence: Synthetic peptide
HSTGPTR

SEQ ID NO: 182 - protein, 7 aa, Artificial Sequence
  Description of Artificial Sequence: Synthetic peptide
HTLLSTT

SEQ ID NO: 183 - protein, 7 aa, Artificial Sequence
  Description of Artificial Sequence: Synthetic peptide
HYPTVNF

SEQ ID NO: 184 - protein, 12 aa, Artificial Sequence
  Description of Artificial Sequence: Synthetic peptide
IAHVPETRLAQM

SEQ ID NO: 185 - protein, 12 aa, Artificial Sequence
  Description of Artificial Sequence: Synthetic peptide
IFSMGTALARPL

SEQ ID NO: 186 - protein, 7 aa, Artificial Sequence
  Description of Artificial Sequence: Synthetic peptide
IGYPVLP

SEQ ID NO: 187 - protein, 12 aa, Artificial Sequence
  Description of Artificial Sequence: Synthetic peptide
INFQFLKPSTTR

SEQ ID NO: 188 - protein, 12 aa, Artificial Sequence
  Description of Artificial Sequence: Synthetic peptide
INKHPQQVSTLL

SEQ ID NO: 189 - protein, 7 aa, Artificial Sequence
  Description of Artificial Sequence: Synthetic peptide
IQHQAKT

SEQ ID NO: 190 - protein, 7 aa, Artificial Sequence
  Description of Artificial Sequence: Synthetic peptide
  MOD_RES (3)..(3): Any amino acid
  MOD_RES (5)..(5): Any amino acid
IRXLXIS

SEQ ID NO: 191 - protein, 12 aa, Artificial Sequence
  Description of Artificial Sequence: Synthetic peptide
ISPSHSQAQADL

SEQ ID NO: 192 - protein, 7 aa, Artificial Sequence
  Description of Artificial Sequence: Synthetic peptide
KAFDKHG

SEQ ID NO: 193 - protein, 7 aa, Artificial Sequence
  Description of Artificial Sequence: Synthetic peptide
KATITGM

SEQ ID NO: 194 - protein, 12 aa, Artificial Sequence
  Description of Artificial Sequence: Synthetic peptide
KEIPPIPLLAPS

SEQ ID NO: 195 - protein, 16 aa, Artificial Sequence
  Description of Artificial Sequence: Synthetic peptide
KEIPPIPLLAPSGGGS

SEQ ID NO: 196 - protein, 20 aa, Artificial Sequence
  Description of Artificial Sequence: Synthetic peptide
KIPKACCVPTELSAISMLYL

SEQ ID NO: 197 - protein, 33 aa, Artificial Sequence
  Description of Artificial Sequence: Synthetic polypeptide
KIPKASSVPTELSAIATLYLAAAAEPRRAVAAL

SEQ ID NO: 198 - protein, 20 aa, Artificial Sequence
  Description of Artificial Sequence: Synthetic peptide
KIPKASSVPTELSAISTLYL

SEQ ID NO: 199 - protein, 33 aa, Artificial Sequence
  Description of Artificial Sequence: Synthetic polypeptide
KIPKASSVPTELSAISTLYLAAAAEPRRAVAAL

SEQ ID NO: 200 - protein, 33 aa, Artificial Sequence
  Description of Artificial Sequence: Synthetic polypeptide
KIPKASSVPTELSAISTLYLAAAAEPRRAVAEL

SEQ ID NO: 201 - protein, 33 aa, Artificial Sequence
  Description of Artificial Sequence: Synthetic polypeptide
KIPKASSVPTELSAISTLYLAAAAEPRREVAEL

SEQ ID NO: 202 - protein, 33 aa, Artificial Sequence
  Description of Artificial Sequence: Synthetic polypeptide
  MOD_RES (25)..(25): Any amino acid
  MOD_RES (29)..(29): Any amino acid
  MOD_RES (32)..(32): Any amino acid
KIPKASSVPTELSAISTLYLAAAAXPRRXVAXL

SEQ ID NO: 203 - protein, 29 aa, Artificial Sequence
  Description of Artificial Sequence: Synthetic peptide
  MOD_RES (21)..(21): Any amino acid
  MOD_RES (25)..(25): Any amino acid
  MOD_RES (28)..(28): Any amino acid
KIPKASSVPTELSAISTLYLXPRRXVAXL

SEQ ID NO: 204 - protein, 7 aa, Artificial Sequence
  Description of Artificial Sequence: Synthetic peptide
KLHASLA

SEQ ID NO: 205 - protein, 7 aa, Artificial Sequence
  Description of Artificial Sequence: Synthetic peptide
KLSAWSF

SEQ ID NO: 206 - protein, 15 aa, Artificial Sequence
  Description of Artificial Sequence: Synthetic peptide
KLTWQELYQLKYKGI

SEQ ID NO: 207 - protein, 31 aa, Artificial Sequence
  Description of Artificial Sequence: Synthetic polypeptide
KLTWQELYQLKYKGIGGGAAAAEPRREVAEL

SEQ ID NO: 208 - protein, 7 aa, Artificial Sequence
  Description of Artificial Sequence: Synthetic peptide
KMNHMPN

SEQ ID NO: 209 - protein, 7 aa, Artificial Sequence
  Description of Artificial Sequence: Synthetic peptide
KPMQFVH

SEQ ID NO: 210 - protein, 7 aa, Artificial Sequence
  Description of Artificial Sequence: Synthetic peptide
KTSSWAN

SEQ ID NO: 211 - protein, 7 aa, Artificial Sequence
  Description of Artificial Sequence: Synthetic peptide
LASTTHV

SEQ ID NO: 212 - protein, 12 aa, Artificial Sequence
  Description of Artificial Sequence: Synthetic peptide
LDYPIPQTVLHH

SEQ ID NO: 213 - protein, 12 aa, Artificial Sequence
  Description of Artificial Sequence: Synthetic peptide
LFAAVPSTQFFR

SEQ ID NO: 214 - protein, 12 aa, Artificial Sequence
  Description of Artificial Sequence: Synthetic peptide
LGFDPTSTRFYT

SEQ ID NO: 215 - protein, 7 aa, Artificial Sequence
  Description of Artificial Sequence: Synthetic peptide
LGPGKAF

SEQ ID NO: 216 - protein, 7 aa, Artificial Sequence
  Description of Artificial Sequence: Synthetic peptide
LKPFSGA

SEQ ID NO: 217 - protein, 12 aa, Artificial Sequence
  Description of Artificial Sequence: Synthetic peptide
LLADTTHHRPWP

SEQ ID NO: 218 - protein, 12 aa, Artificial Sequence
  Description of Artificial Sequence: Synthetic peptide
LLADTTHHRPWT

SEQ ID NO: 219 - protein, 16 aa, Artificial Sequence
  Description of Artificial Sequence: Synthetic peptide
LLADTTHHRPWTGGGS

SEQ ID NO: 220 - protein, 7 aa, Artificial Sequence
  Description of Artificial Sequence: Synthetic peptide
LLPLKFK

SEQ ID NO: 221 - protein, 7 aa, Artificial Sequence
  Description of Artificial Sequence: Synthetic peptide
LPFQPPI

SEQ ID NO: 222 - protein, 7 aa, Artificial Sequence
  Description of Artificial Sequence: Synthetic peptide
LPLTPLP

SEQ ID NO: 223 - protein, 12 aa, Artificial Sequence
  Description of Artificial Sequence: Synthetic peptide
LPRDLHATPQQI

SEQ ID NO: 224 - protein, 7 aa, Artificial Sequence
  Description of Artificial Sequence: Synthetic peptide
LPSIHNL

SEQ ID NO: 225 - protein, 12 aa, Artificial Sequence
  Description of Artificial Sequence: Synthetic peptide
LPWAPNLPDSTA

SEQ ID NO: 226 - protein, 12 aa, Artificial Sequence
  Description of Artificial Sequence: Synthetic peptide
LPWTEPSFWRTP

SEQ ID NO: 227 - protein, 16 aa, Artificial Sequence
  Description of Artificial Sequence: Synthetic peptide
LPWTEPSFWRTPGGGS

SEQ ID NO: 228 - protein, 7 aa, Artificial Sequence
  Description of Artificial Sequence: Synthetic peptide
LQKSPSL

SEQ ID NO: 229 - protein, 12 aa, Artificial Sequence
  Description of Artificial Sequence: Synthetic peptide
LQPSQPQRFAPT

SEQ ID NO: 230 - protein, 12 aa, Artificial Sequence
  Description of Artificial Sequence: Synthetic peptide
LRAFPSLPHTVT

SEQ ID NO: 231 - protein, 7 aa, Artificial Sequence
  Description of Artificial Sequence: Synthetic peptide
LSAPMEY

SEQ ID NO: 232 - protein, 7 aa, Artificial Sequence
  Description of Artificial Sequence: Synthetic peptide
LSKNPLL

SEQ ID NO: 233 - protein, 12 aa, Artificial Sequence
  Description of Artificial Sequence: Synthetic peptide
LSLRASAATDFQ

SEQ ID NO: 234 - protein, 12 aa, Artificial Sequence
  Description of Artificial Sequence: Synthetic peptide
LSPPMQLQPTYS

SEQ ID NO: 235 - protein, 12 aa, Artificial Sequence
  Description of Artificial Sequence: Synthetic peptide
LTPTMFNMHGVL

SEQ ID NO: 236 - protein, 7 aa, Artificial Sequence
  Description of Artificial Sequence: Synthetic peptide
LTQTLQY

SEQ ID NO: 237 - protein, 12 aa, Artificial Sequence
  Description of Artificial Sequence: Synthetic peptide
MHNVSDSNDSAI

SEQ ID NO: 238 - protein, 7 aa, Artificial Sequence
  Description of Artificial Sequence: Synthetic peptide
MKVHERS

SEQ ID NO: 239 - protein, 12 aa, Artificial Sequence
  Description of Artificial Sequence: Synthetic peptide
MPQTLVLPRSLL

SEQ ID NO: 240 - protein, 12 aa, Artificial Sequence
  Description of Artificial Sequence: Synthetic peptide
MQFTPAPSPSDH

SEQ ID NO: 241 - protein, 7 aa, Artificial Sequence
  Description of Artificial Sequence: Synthetic peptide
MTSQTLR

SEQ ID NO: 242 - protein, 7 aa, Artificial Sequence
  Description of Artificial Sequence: Synthetic peptide
MYPLPAP

SEQ ID NO: 243 - protein, 7 aa, Artificial Sequence
  Description of Artificial Sequence: Synthetic peptide
NERQMEL

SEQ ID NO: 244 - protein, 7 aa, Artificial Sequence
  Description of Artificial Sequence: Synthetic peptide
NFAMNLR

SEQ ID NO: 245 - protein, 7 aa, Artificial Sequence
  Description of Artificial Sequence: Synthetic peptide
NITQLGS

SEQ ID NO: 246 - protein, 7 aa, Artificial Sequence
  Description of Artificial Sequence: Synthetic peptide
NKPLSTL

SEQ ID NO: 247 - protein, 12 aa, Artificial Sequence
  Description of Artificial Sequence: Synthetic peptide
NNVSQKWQQRLI

SEQ ID NO: 248 - protein, 16 aa, Artificial Sequence
  Description of Artificial Sequence: Synthetic peptide
NNVSQKWQQRLIGGGS

SEQ ID NO: 249 - protein, 16 aa, Artificial Sequence
  Description of Artificial Sequence: Synthetic peptide
NPDHPDIPQDVHGGGK

SEQ ID NO: 250 - protein, 7 aa, Artificial Sequence
  Description of Artificial Sequence: Synthetic peptide
NPMIMNQ

SEQ ID NO: 251 - protein, 7 aa, Artificial Sequence
  Description of Artificial Sequence: Synthetic peptide
NPQMQRS

SEQ ID NO: 252 - protein, 7 aa, Artificial Sequence
  Description of Artificial Sequence: Synthetic peptide
NPRSQAT

SEQ ID NO: 253 - protein, 16 aa, Artificial Sequence
  Description of Artificial Sequence: Synthetic peptide
NPYAPTIPQSVAGGGK

SEQ ID NO: 254 - protein, 12 aa, Artificial Sequence
  Description of Artificial Sequence: Synthetic peptide
NPYHPTIPQSVH

SEQ ID NO: 255 - protein, 16 aa, Artificial Sequence
  Description of Artificial Sequence: Synthetic peptide
NPYHPTIPQSVHGGGK

SEQ ID NO: 256 - protein, 12 aa, Artificial Sequence
  Description of Artificial Sequence: Synthetic peptide
NSMIAHNKTRMH

SEQ ID NO: 257 - protein, 16 aa, Artificial Sequence
  Description of Artificial Sequence: Synthetic peptide
NSMIAHNKTRMHGGGS

SEQ ID NO: 258 - protein, 12 aa, Artificial Sequence
  Description of Artificial Sequence: Synthetic peptide
NSSMLGMLPSSF

SEQ ID NO: 259 - protein, 12 aa, Artificial Sequence
  Description of Artificial Sequence: Synthetic peptide
NTSSSQGTQRLG

SEQ ID NO: 260 - protein, 12 aa, Artificial Sequence
  Description of Artificial Sequence: Synthetic peptide
NTTTDIPSPSQF

SEQ ID NO: 261 - protein, 7 aa, Artificial Sequence
  Description of Artificial Sequence: Synthetic peptide
NYPTLKS

SEQ ID NO: 262 - protein, 12 aa, Artificial Sequence
  Description of Artificial Sequence: Synthetic peptide
NYSHLRVKLPTP

SEQ ID NO: 263 - protein, 16 aa, Artificial Sequence
  Description of Artificial Sequence: Synthetic peptide
NYSHLRVKLPTPGGGS

SEQ ID NO: 264 - protein, 7 aa, Artificial Sequence
  Description of Artificial Sequence: Synthetic peptide
PAKQKAH

SEQ ID NO: 265 - protein, 7 aa, Artificial Sequence
  Description of Artificial Sequence: Synthetic peptide
PDIPLSR

SEQ ID NO: 266 - protein, 12 aa, Artificial Sequence
  Description of Artificial Sequence: Synthetic peptide
PGQWPSSLTLYK

SEQ ID NO: 267 - protein, 7 aa, Artificial Sequence
  Description of Artificial Sequence: Synthetic peptide
PHNPGKL

SEQ ID NO: 268 - protein, 7 aa, Artificial Sequence
  Description of Artificial Sequence: Synthetic peptide
PIDAFFD

SEQ ID NO: 269 - protein, 7 aa, Artificial Sequence
  Description of Artificial Sequence: Synthetic peptide
PLTQPSH

SEQ ID NO: 270 - protein, 7 aa, Artificial Sequence
  Description of Artificial Sequence: Synthetic peptide
PPKDSRG

SEQ ID NO: 271 - protein, 7 aa, Artificial Sequence
  Description of Artificial Sequence: Synthetic peptide
PPNMARA

SEQ ID NO: 272 - protein, 7 aa, Artificial Sequence
  Description of Artificial Sequence: Synthetic peptide
PSMKHWR

SEQ ID NO: 273 - protein, 7 aa, Artificial Sequence
  Description of Artificial Sequence: Synthetic peptide
PTNKPHT

SEQ ID NO: 274 - protein, 7 aa, Artificial Sequence
  Description of Artificial Sequence: Synthetic peptide
PTTMTRW

SEQ ID NO: 275 - protein, 7 aa, Artificial Sequence
  Description of Artificial Sequence: Synthetic peptide
PTTWGHL

SEQ ID NO: 276 - protein, 36 aa, Artificial Sequence
  Description of Artificial Sequence: Synthetic polypeptide
  MOD_RES (2)..(2): Any amino acid
  MOD_RES (5)..(5): Any amino acid
  MOD_RES (8)..(8): Any amino acid
  MOD_RES (11)..(11): Any amino acid
  MOD_RES (14)..(14): Any amino acid
  MOD_RES (17)..(17): Any amino acid
  MOD_RES (20)..(20): Any amino acid
  MOD_RES (23)..(23): Any amino acid
  MOD_RES (26)..(26): Any amino acid
  MOD_RES (29)..(29): Any amino acid
  MOD_RES (32)..(32): Any amino acid
  MOD_RES (35)..(35): Any amino acid
PXGPXGPXGPXGPXGPXAPXGPXGPXGPXGPXGPXG

SEQ ID NO: 277 - protein, 12 aa, Artificial Sequence
  Description of Artificial Sequence: Synthetic peptide
QHNFRGASSSAP

SEQ ID NO: 278 - protein, 12 aa, Artificial Sequence
  Description of Artificial Sequence: Synthetic peptide
QIPQMRILHPYG

SEQ ID NO: 279 - protein, 12 aa, Artificial Sequence
  Description of Artificial Sequence: Synthetic peptide
QIQKPPRTPPSL

SEQ ID NO: 280 - protein, 12 aa, Artificial Sequence
  Description of Artificial Sequence: Synthetic peptide
QLTQTMWKDTTL

SEQ ID NO: 281 - protein, 12 aa, Artificial Sequence
  Description of Artificial Sequence: Synthetic peptide
QNLPPERYSEAT

SEQ ID NO: 282 - protein, 7 aa, Artificial Sequence
  Description of Artificial Sequence: Synthetic peptide
QNPRQIY

SEQ ID NO: 283 - protein, 7 aa, Artificial Sequence
  Description of Artificial Sequence: Synthetic peptide
QNYLLPK

SEQ ID NO: 284 - protein, 7 aa, Artificial Sequence
  Description of Artificial Sequence: Synthetic peptide
QPGLWPS

SEQ ID NO: 285 - protein, 12 aa, Artificial Sequence
  Description of Artificial Sequence: Synthetic peptide
QRSWTLDSALSM

SEQ ID NO: 286 - protein, 16 aa, Artificial Sequence
  Description of Artificial Sequence: Synthetic peptide
QRSWTLDSALSMGGGS

SEQ ID NO: 287 - protein, 12 aa, Artificial Sequence
  Description of Artificial Sequence: Synthetic peptide
QSLSFAGPPAWQ

SEQ ID NO: 288 - protein, 7 aa, Artificial Sequence
  Description of Artificial Sequence: Synthetic peptide
QSSYNPI

SEQ ID NO: 289 - protein, 7 aa, Artificial Sequence
  Description of Artificial Sequence: Synthetic peptide
QTHARHQ

SEQ ID NO: 290 - protein, 7 aa, Artificial Sequence
  Description of Artificial Sequence: Synthetic peptide
QTHSSLW

SEQ ID NO: 291 - protein, 12 aa, Artificial Sequence
  Description of Artificial Sequence: Synthetic peptide
QTTMTPLWPSFS

SEQ ID NO: 292 - protein, 12 aa, Artificial Sequence
  Description of Artificial Sequence: Synthetic peptide
RCMSEVISFNCP

SEQ ID NO: 293 - protein, 7 aa, Artificial Sequence
  Description of Artificial Sequence: Synthetic peptide
RHTLPLH

SEQ ID NO: 294 - protein, 7 aa, Artificial Sequence
  Description of Artificial Sequence: Synthetic peptide
RPHTITN

SEQ ID NO: 295 - protein, 12 aa, Artificial Sequence
  Description of Artificial Sequence: Synthetic peptide
RSPYYNKWSSKF

SEQ ID NO: 296 - protein, 12 aa, Artificial Sequence
  Description of Artificial Sequence: Synthetic peptide
RTPLQPLEDFRP

SEQ ID NO: 297 - protein, 12 aa, Artificial Sequence
  Description of Artificial Sequence: Synthetic peptide
SAGHIHEAHRPL

SEQ ID NO: 298 - protein, 12 aa, Artificial Sequence
  Description of Artificial Sequence: Synthetic peptide
SAISDHRAHRSH

SEQ ID NO: 299 - protein, 7 aa, Artificial Sequence
  Description of Artificial Sequence: Synthetic peptide
SAKGRAD

SEQ ID NO: 300 - protein, 7 aa, Artificial Sequence
  Description of Artificial Sequence: Synthetic peptide
SAKKVFS

SEQ ID NO: 301 - protein, 7 aa, Artificial Sequence
  Description of Artificial Sequence: Synthetic peptide
SASGTPS

SEQ ID NO: 302 - protein, 12 aa, Artificial Sequence
  Description of Artificial Sequence: Synthetic peptide
SEPTYWRPNMSG

SEQ ID NO: 303 - protein, 12 aa, Artificial Sequence
  Description of Artificial Sequence: Synthetic peptide
SFAPDIKYPVPS

SEQ ID NO: 304 - protein, 12 aa, Artificial Sequence
  Description of Artificial Sequence: Synthetic peptide
SFQSMSLMTLVV

SEQ ID NO: 305 - protein, 12 aa, Artificial Sequence
  Description of Artificial Sequence: Synthetic peptide
SFWHHHSPRSPL

SEQ ID NO: 306 - protein, 12 aa, Artificial Sequence
  Description of Artificial Sequence: Synthetic peptide
SGHQLLLNKMPN

SEQ ID NO: 307 - protein, 16 aa, Artificial Sequence
  Description of Artificial Sequence: Synthetic peptide
SGHQLLLNKMPNGGGS

SEQ ID NO: 308 - protein, 12 aa, Artificial Sequence
  Description of Artificial Sequence: Synthetic peptide
SIFAHQTPTHKN

SEQ ID NO: 309 - protein, 12 aa, Artificial Sequence
  Description of Artificial Sequence: Synthetic peptide
SIPKMIPTESLL

SEQ ID NO: 310 - protein, 12 aa, Artificial Sequence
  Description of Artificial Sequence: Synthetic peptide
SIPSHSIHSAKA

SEQ ID NO: 311 - protein, 12 aa, Artificial Sequence
  Description of Artificial Sequence: Synthetic peptide
SIRTSMNPPNLL

SEQ ID NO: 312 - protein, 7 aa, Artificial Sequence
  Description of Artificial Sequence: Synthetic peptide
SKTSSTS

SEQ ID NO: 313 - protein, 7 aa, Artificial Sequence
  Description of Artificial Sequence: Synthetic peptide
SLLTPWL

SEQ ID NO: 314 - protein, 12 aa, Artificial Sequence
  Description of Artificial Sequence: Synthetic peptide
SLPHYIDNPFRQ

SEQ ID NO: 315 - protein, 12 aa, Artificial Sequence
  Description of Artificial Sequence: Synthetic peptide
SLSKANILHLYG

SEQ ID NO: 316 - protein, 12 aa, Artificial Sequence
  Description of Artificial Sequence: Synthetic peptide
SLVTADASFTPS

SEQ ID NO: 317 - protein, 7 aa, Artificial Sequence
  Description of Artificial Sequence: Synthetic peptide
SMAAKSS

SEQ ID NO: 318 - protein, 12 aa, Artificial Sequence
  Description of Artificial Sequence: Synthetic peptide
SMVYGNRLPSAL

SEQ ID NO: 319 - protein, 7 aa, Artificial Sequence
  Description of Artificial Sequence: Synthetic peptide
SMYDTHS

SEQ ID NO: 320 - protein, 7 aa, Artificial Sequence
  Description of Artificial Sequence: Synthetic peptide
SPEMKPR

SEQ ID NO: 321 - protein, 12 aa, Artificial Sequence
  Description of Artificial Sequence: Synthetic peptide
SPNFSWLPLGTT

SEQ ID NO: 322 - protein, 12 aa, Artificial Sequence
  Description of Artificial Sequence: Synthetic peptide
SPNLPWSKLSAY

SEQ ID NO: 323 - protein, 7 aa, Artificial Sequence
  Description of Artificial Sequence: Synthetic peptide
SPNNPRE

SEQ ID NO: 324 - protein, 7 aa, Artificial Sequence
  Description of Artificial Sequence: Synthetic peptide
SPNNTRE

SEQ ID NO: 325 - protein, 12 aa, Artificial Sequence
  Description of Artificial Sequence: Synthetic peptide
SPSLMARSSPYW

SEQ ID NO: 326 - protein, 7 aa, Artificial Sequence
  Description of Artificial Sequence: Synthetic peptide
SQHSTQD

SEQ ID NO: 327 - protein, 12 aa, Artificial Sequence
  Description of Artificial Sequence: Synthetic peptide
SQTLPYSNAPSP

SEQ ID NO: 328 - protein, 7 aa, Artificial Sequence
  Description of Artificial Sequence: Synthetic peptide
SRTGAHH

SEQ ID NO: 329 - protein, 7 aa, Artificial Sequence
  Description of Artificial Sequence: Synthetic peptide
SSHHHRH

SEQ ID NO: 330 - protein, 7 aa, Artificial Sequence
  Description of Artificial Sequence: Synthetic peptide
SSPPRVY

SEQ ID NO: 331 - protein, 7 aa, Artificial Sequence
  Description of Artificial Sequence: Synthetic peptide
SSSMAKM

SEQ ID NO: 332 - protein, 12 aa, Artificial Sequence
  Description of Artificial Sequence: Synthetic peptide
SSTLKTFFGFPD

SEQ ID NO: 333 - protein, 16 aa, Artificial Sequence
  Description of Artificial Sequence: Synthetic peptide
SSTLKTFFGFPDGGGS

SEQ ID NO: 334 - protein, 12 aa, Artificial Sequence
  Description of Artificial Sequence: Synthetic peptide
SSTQAHPFAPQL

SEQ ID NO: 335 - protein, 12 aa, Artificial Sequence
  Description of Artificial Sequence: Synthetic peptide
SSTQVQHTLLQT

SEQ ID NO: 336 - protein, 7 aa, Artificial Sequence
  Description of Artificial Sequence: Synthetic peptide
SSVPGRP

SEQ ID NO: 337 - protein, 7 aa, Artificial Sequence
  Description of Artificial Sequence: Synthetic peptide
SSYEYHA

SEQ ID NO: 338 - protein, 7 aa, Artificial Sequence
  Description of Artificial Sequence: Synthetic peptide
STLASMR

SEQ ID NO: 339 - protein, 12 aa, Artificial Sequence
  Description of Artificial Sequence: Synthetic peptide
STPNSYSLPQAR

SEQ ID NO: 340 - protein, 7 aa, Artificial Sequence
  Description of Artificial Sequence: Synthetic peptide
STQAHPW

SEQ ID NO: 341 - protein, 7 aa, Artificial Sequence
  Description of Artificial Sequence: Synthetic peptide
STSAKHW

SEQ ID NO: 342 - protein, 12 aa, Artificial Sequence
  Description of Artificial Sequence: Synthetic peptide
STVVMQPPPRPA

SEQ ID NO: 343 - protein, 12 aa, Artificial Sequence
  Description of Artificial Sequence: Synthetic peptide
SVFLPTRHSPDL

SEQ ID NO: 344 - protein, 12 aa, Artificial Sequence
  Description of Artificial Sequence: Synthetic peptide
SVQTRPLFHSHF

SEQ ID NO: 345 - protein, 12 aa, Artificial Sequence
  Description of Artificial Sequence: Synthetic peptide
SVSVGMKPSPRP

SEQ ID NO: 346 - protein, 12 aa, Artificial Sequence
  Description of Artificial Sequence: Synthetic peptide
  MOD_RES (11)..(11): Any amino acid
SVSVGMNAESXA

SEQ ID NO: 347 - protein, 12 aa, Artificial Sequence
  Description of Artificial Sequence: Synthetic peptide
SVSVGMNAESYG

SEQ ID NO: 348 - protein, 12 aa, Artificial Sequence
  Description of Artificial Sequence: Synthetic peptide
  MOD_RES (11)..(11): Any amino acid
SVSVGTEAESXA

SEQ ID NO: 349 - protein, 12 aa, Artificial Sequence
  Description of Artificial Sequence: Synthetic peptide
SWPLYSRDSGLG

SEQ ID NO: 350 - protein, 12 aa, Artificial Sequence
  Description of Artificial Sequence: Synthetic peptide
SYIDSMVPSTQT

SEQ ID NO: 351 - protein, 12 aa, Artificial Sequence
  Description of Artificial Sequence: Synthetic peptide
SYKTTDSDTSPL

SEQ ID NO: 352 - protein, 16 aa, Artificial Sequence
  Description of Artificial Sequence: Synthetic peptide
SYSQMDPPRSLPGGGS

SEQ ID NO: 353 - protein, 12 aa, Artificial Sequence
  Description of Artificial Sequence: Synthetic peptide
TAAASNLRAVPP

SEQ ID NO: 354 - protein, 12 aa, Artificial Sequence
  Description of Artificial Sequence: Synthetic peptide
TAPLSHPPRPGA

SEQ ID NO: 355 - protein, 7 aa, Artificial Sequence
  Description of Artificial Sequence: Synthetic peptide
TDHPPKA

SEQ ID NO: 356 - protein, 7 aa, Artificial Sequence
  Description of Artificial Sequence: Synthetic peptide
TGLAKTA

SEQ ID NO: 357 - protein, 12 aa, Artificial Sequence
  Description of Artificial Sequence: Synthetic peptide
TGLLPNSSGAGI

SEQ ID NO: 358 - protein, 12 aa, Artificial Sequence
  Description of Artificial Sequence: Synthetic peptide
TGPPSRQPAPLH

SEQ ID NO: 359 - protein, 7 aa, Artificial Sequence
  Description of Artificial Sequence: Synthetic peptide
TGPTSLS

SEQ ID NO: 360 - protein, 12 aa, Artificial Sequence
  Description of Artificial Sequence: Synthetic peptide
THPVVFEDERLF

SEQ ID NO: 361 - protein, 7 aa, Artificial Sequence
  Description of Artificial Sequence: Synthetic peptide
TIHSKPA

SEQ ID NO: 362 - protein, 7 aa, Artificial Sequence
  Description of Artificial Sequence: Synthetic peptide
TKDWLPS

SEQ ID NO: 363 - protein, 7 aa, Artificial Sequence
  Description of Artificial Sequence: Synthetic peptide
TLAFQTA

SEQ ID NO: 364 - protein, 7 aa, Artificial Sequence
  Description of Artificial Sequence: Synthetic peptide
TLAPTFR

SEQ ID NO: 365 - protein, 12 aa, Artificial Sequence
  Description of Artificial Sequence: Synthetic peptide
TLDKYTRLLSRY

SEQ ID NO: 366 - protein, 7 aa, Artificial Sequence
  Description of Artificial Sequence: Synthetic peptide
TLGLPML

SEQ ID NO: 367 - protein, 7 aa, Artificial Sequence
  Description of Artificial Sequence: Synthetic peptide
TLLRTQV

SEQ ID NO: 368 - protein, 7 aa, Artificial Sequence
  Description of Artificial Sequence: Synthetic peptide
TLMTTPP

SEQ ID NO: 369 - protein, 12 aa, Artificial Sequence
  Description of Artificial Sequence: Synthetic peptide
TLPSPLALLTVH

SEQ ID NO: 370 - protein, 7 aa, Artificial Sequence
  Description of Artificial Sequence: Synthetic peptide
TLQRMGQ

SEQ ID NO: 371 - protein, 12 aa, Artificial Sequence
  Description of Artificial Sequence: Synthetic peptide
TLSNGHRYLELL

SEQ ID NO: 372 - protein, 12 aa, Artificial Sequence
  Description of Artificial Sequence: Synthetic peptide
TMGFTAPRFPHY

SEQ ID NO: 373 - protein, 12 aa, Artificial Sequence
  Description of Artificial Sequence: Synthetic peptide
TMRNPITSLISV

SEQ ID NO: 374 - protein, 16 aa, Artificial Sequence
  Description of Artificial Sequence: Synthetic peptide
TMRNPITSLISVGGGS

SEQ ID NO: 375 - protein, 7 aa, Artificial Sequence
  Description of Artificial Sequence: Synthetic peptide
TMTNMAK

SEQ ID NO: 376 - protein, 12 aa, Artificial Sequence
  Description of Artificial Sequence: Synthetic peptide
TPLSYLKGLVTV

SEQ ID NO: 377 - protein, 12 aa, Artificial Sequence
  Description of Artificial Sequence: Synthetic peptide
TPLTSPSLVRPQ

SEQ ID NO: 378 - protein, 12 aa, Artificial Sequence
  Description of Artificial Sequence: Synthetic peptide
TPSPKLLQVFQA

SEQ ID NO: 379 - protein, 12 aa, Artificial Sequence
  Description of Artificial Sequence: Synthetic peptide
TPSTGLGMSPAV

SEQ ID NO: 380 - protein, 12 aa, Artificial Sequence
  Description of Artificial Sequence: Synthetic peptide
TPVYSLKLGPWP

SEQ ID NO: 381 - protein, 12 aa, Artificial Sequence
  Description of Artificial Sequence: Synthetic peptide
TQTWPQSSSHGL

SEQ ID NO: 382 - protein, 7 aa, Artificial Sequence
  Description of Artificial Sequence: Synthetic peptide
TRFYDSL

SEQ ID NO: 383 - protein, 12 aa, Artificial Sequence
  Description of Artificial Sequence: Synthetic peptide
TRLVPSRYYHHP

SEQ ID NO: 384 - protein, 12 aa, Artificial Sequence
  Description of Artificial Sequence: Synthetic peptide
TSPIPQMRTVPP

SEQ ID NO: 385 - protein, 7 aa, Artificial Sequence
  Description of Artificial Sequence: Synthetic peptide
TTKNFNK

SEQ ID NO: 386 - protein, 7 aa, Artificial Sequence
  Description of Artificial Sequence: Synthetic peptide
TTLSPRT

SEQ ID NO: 387 - protein, 12 aa, Artificial Sequence
  Description of Artificial Sequence: Synthetic peptide
TTNSSMTMQLQR

SEQ ID NO: 388 - protein, 12 aa, Artificial Sequence
  Description of Artificial Sequence: Synthetic peptide
TTTLPVQPTLRN

SEQ ID NO: 389 - protein, 12 aa, Artificial Sequence
  Description of Artificial Sequence: Synthetic peptide
TTTWTTTARWPL

SEQ ID NO: 390 - protein, 7 aa, Artificial Sequence
  Description of Artificial Sequence: Synthetic peptide
TTYNSPP

SEQ ID NO: 391 - protein, 12 aa, Artificial Sequence
  Description of Artificial Sequence: Synthetic peptide
TVAQMPPHWQLT

SEQ ID NO: 392 - protein, 7 aa, Artificial Sequence
  Description of Artificial Sequence: Synthetic peptide
TVLGTFP

SEQ ID NO: 393 - protein, 12 aa, Artificial Sequence
  Description of Artificial Sequence: Synthetic peptide
TWNSNSTQYGNR

SEQ ID NO: 394 - protein, 12 aa, Artificial Sequence
  Description of Artificial Sequence: Synthetic peptide
TWTLPAMHPRPA

SEQ ID NO: 395 - protein, 7 aa, Artificial Sequence
  Description of Artificial Sequence: Synthetic peptide
VHLTHGQ

SEQ ID NO: 396 - protein, 7 aa, Artificial Sequence
  Description of Artificial Sequence: Synthetic peptide
VHPRPSL

SEQ ID NO: 397 - protein, 12 aa, Artificial Sequence
  Description of Artificial Sequence: Synthetic peptide
VHTSLLQKHPLP

SEQ ID NO: 398 - protein, 11 aa, Artificial Sequence
  Description of Artificial Sequence: Synthetic peptide
VLPNIYMTLSA

SEQ ID NO: 399 - protein, 12 aa, Artificial Sequence
  Description of Artificial Sequence: Synthetic peptide
VMDFASPAHVLP

SEQ ID NO: 400 - protein, 12 aa, Artificial Sequence
  Description of Artificial Sequence: Synthetic peptide
VNQEYWFFPRRP

SEQ ID NO: 401 - protein, 15 aa, Artificial Sequence
  Description of Artificial Sequence: Synthetic peptide
  MOD_RES (6)..(6): Any amino acid
  MOD_RES (11)..(11): Any amino acid
  MOD_RES (14)..(14): Any amino acid
VPPISXTFLFXSTXS

SEQ ID NO: 402 - protein, 12 aa, Artificial Sequence
  Description of Artificial Sequence: Synthetic peptide
  MOD_RES (11)..(11): Any amino acid
VPPLHPALSRXN

SEQ ID NO: 403 - protein, 12 aa, Artificial Sequence
  Description of Artificial Sequence: Synthetic peptide
VSPFLSPTPLLF

SEQ ID NO: 404 - protein, 12 aa, Artificial Sequence
  Description of Artificial Sequence: Synthetic peptide
VSRLGTPSMHPS

SEQ ID NO: 405 - protein, 7 aa, Artificial Sequence
  Description of Artificial Sequence: Synthetic peptide
VVKSNGE

SEQ ID NO: 406 - protein, 11 aa, Artificial Sequence
  Description of Artificial Sequence: Synthetic peptide
VYSSPLSQLPR

SEQ ID NO: 407 - protein, 7 aa, Artificial Sequence
  Description of Artificial Sequence: Synthetic peptide
WLPPRTQ

SEQ ID NO: 408 - protein, 12 aa, Artificial Sequence
  Description of Artificial Sequence: Synthetic peptide
WPANKLSTKSMY

SEQ ID NO: 409 - protein, 12 aa, Artificial Sequence
  Description of Artificial Sequence: Synthetic peptide
WPFNHFPWWNVP

SEQ ID NO: 410 - protein, 12 aa, Artificial Sequence
  Description of Artificial Sequence: Synthetic peptide
WPTYLNPSSLKA

SEQ ID NO: 411 - protein, 12 aa, Artificial Sequence
  Description of Artificial Sequence: Synthetic peptide
WSAHIVPYSHKP

SEQ ID NO: 412 - protein, 12 aa, Artificial Sequence
  Description of Artificial Sequence: Synthetic peptide
WWPNSLNWVPRP

SEQ ID NO: 413 - protein, 7 aa, Artificial Sequence
  Description of Artificial Sequence: Synthetic peptide
WYPNHLA

SEQ ID NO: 414 - protein, 7 aa, Artificial Sequence
  Description of Artificial Sequence: Synthetic peptide
  MOD_RES (1)..(1): Any amino acid
  MOD_RES (4)..(4): Any amino acid
XITXGAY

SEQ ID NO: 415 - protein, 9 aa, Artificial Sequence
  Description of Artificial Sequence: Synthetic peptide
  MOD_RES (1)..(1): Any amino acid
XPRRAVAAL

SEQ ID NO: 416 - protein, 9 aa, Artificial Sequence
  Description of Artificial Sequence: Synthetic peptide
  MOD_RES (1)..(1): Any amino acid
  MOD_RES (8)..(8): Any amino acid
XPRRAVAXL

SEQ ID NO: 417 - protein, 9 aa, Artificial Sequence
  Description of Artificial Sequence: Synthetic peptide
  MOD_RES (1)..(1): Any amino acid
  MOD_RES (5)..(5): Any amino acid
  MOD_RES (8)..(8): Any amino acid
XPRRXVAXL

SEQ ID NO: 418 - protein, 7 aa, Artificial Sequence
  Description of Artificial Sequence: Synthetic peptide
  MOD_RES (1)..(2): Any amino acid
  MOD_RES (6)..(6): Any amino acid
XXFPLXG

SEQ ID NO: 419 - protein, 12 aa, Artificial Sequence
  Description of Artificial Sequence: Synthetic peptide
YATQHNWRLKHE

SEQ ID NO: 420 - protein, 10 aa, Artificial Sequence
  Description of Artificial Sequence: Synthetic peptide
YCPMRLCTDC

SEQ ID NO: 421 - protein, 12 aa, Artificial Sequence
  Description of Artificial Sequence: Synthetic peptide
YELQMPLTLPLN

SEQ ID NO: 422 - protein, 7 aa, Artificial Sequence
  Description of Artificial Sequence: Synthetic peptide
YEPAAAE

SEQ ID NO: 423 - protein, 12 aa, Artificial Sequence
  Description of Artificial Sequence: Synthetic peptide
YGKGFSPYFHVT

SEQ ID NO: 424 - protein, 12 aa, Artificial Sequence
  Description of Artificial Sequence: Synthetic peptide
YPHYSLPGSSTL

SEQ ID NO: 425 - protein, 12 aa, Artificial Sequence
  Description of Artificial Sequence: Synthetic peptide
YPIMSHTCCHGV

SEQ ID NO: 426 - protein, 7 aa, Artificial Sequence
  Description of Artificial Sequence: Synthetic peptide
YPKALRN

SEQ ID NO: 427 - protein, 12 aa, Artificial Sequence
  Description of Artificial Sequence: Synthetic peptide
YPSLLKMQPQFS

SEQ ID NO: 428 - protein, 12 aa, Artificial Sequence
  Description of Artificial Sequence: Synthetic peptide
YQPRPFVTTSPM

SEQ ID NO: 429 - protein, 12 aa, Artificial Sequence
  Description of Artificial Sequence: Synthetic peptide
YSAPLARSNVVM

SEQ ID NO: 430 - protein, 12 aa, Artificial Sequence
  Description of Artificial Sequence: Synthetic peptide
YTRLSHNPYTLS

SEQ ID NO: 431 - protein, 12 aa, Artificial Sequence
  Description of Artificial Sequence: Synthetic peptide
YTTHVLPFAPSS

SEQ ID NO: 432 - protein, 12 aa, Artificial Sequence
  Description of Artificial Sequence: Synthetic peptide
YTWQTIREQYEM

SEQ ID NO: 433 - protein, 152 aa, Artificial Sequence
  Description of Artificial Sequence: Synthetic polypeptide
ASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 434 - protein, 217 aa, Artificial Sequence
  Description of Artificial Sequence: Synthetic polypeptide
MPIGSLLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 435 - protein, 212 aa, Artificial Sequence
  Description of Artificial Sequence: Synthetic polypeptide
LLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 436 - protein, 200 aa, Artificial Sequence
  Description of Artificial Sequence: Synthetic polypeptide
VIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 437 - protein, 188 aa, Artificial Sequence
  Description of Artificial Sequence: Synthetic polypeptide
IIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 438 - protein, 176 aa, Artificial Sequence
  Description of Artificial Sequence: Synthetic polypeptide
GLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 439 - protein, 164 aa, Artificial Sequence
  Description of Artificial Sequence: Synthetic polypeptide
ILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 440 - protein, 115 aa, Artificial Sequence
  Description of Artificial Sequence: Synthetic polypeptide
  MOD_RES (1)..(1): Any amino acid
  See specification as filed for detailed description of substitutions and preferred embodiments
XQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 441 - protein, 153 aa, Artificial Sequence
  Description of Artificial Sequence: Synthetic polypeptide
  MOD_RES (1)..(1): Any amino acid
  See specification as filed for detailed description of substitutions and preferred embodiments
XASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 442 - protein, 53 aa, Artificial Sequence
  Description of Artificial Sequence: Synthetic polypeptide
NSDSECPLSHDGYCLHDGVCMYIEALDKYACNCVVGYIGERCQYRDLKWWELR

SEQ ID NO: 443 - protein, 66 aa, Artificial Sequence
  Description of Artificial Sequence: Synthetic polypeptide
EEAEIPREVIERLARSQIHSIRDLQRLLEIDSVGSEDSLDTSLRAHGVHATKHVPEKRPLPIRRKR

SEQ ID NO: 444 - protein, 70 aa, Artificial Sequence
  Description of Artificial Sequence: Synthetic polypeptide
GPETLCGAELVDALQFVCGDRGFYFNKPTGYGSSSRRAPQTGIVDECCFRSCDLRRLEMYCAPLKPAKSA

SEQ ID NO: 445 - protein, 140 aa, Artificial Sequence
  Description of Artificial Sequence: Synthetic polypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 446 - protein, 146 aa, Artificial Sequence
  Description of Artificial Sequence: Synthetic polypeptide
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 447 - protein, 180 aa, Artificial Sequence
  Description of Artificial Sequence: Synthetic polypeptide
EENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 448 - protein, 137 aa, Artificial Sequence
  Description of Artificial Sequence: Synthetic polypeptide
ENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 449 - protein, 50 aa, Artificial Sequence
  Description of Artificial Sequence: Synthetic polypeptide
VVSHFNDCPDSHTQFCFHGTCRFLVQEDKPACVCHSGYVGARCEHADLLA

SEQ ID NO: 450 - protein, 112 aa, Artificial Sequence
  Description of Artificial Sequence: Synthetic polypeptide
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 451 - protein, 112 aa, Artificial Sequence [sequence omitted]

SEQ ID NO: 452 - protein, 139 aa, Artificial Sequence
  Description of Artificial Sequence: Synthetic polypeptide
AVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 453 - protein, 139 aa, Artificial Sequence
  Description of Artificial Sequence: Synthetic polypeptide
AVRPLRRRQPKKSNELPQANRLPGIFDDVRGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 454 - protein, 114 aa, Artificial Sequence
  Description of Artificial Sequence: Synthetic polypeptide
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 455 - protein, 110 aa, Artificial Sequence
  Description of Artificial Sequence: Synthetic polypeptide
QWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 456 - protein, 116 aa, Artificial Sequence
  Description of Artificial Sequence: Synthetic polypeptide
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 457 - protein, 138 aa, Artificial Sequence
  Description of Artificial Sequence: Synthetic polypeptide
AANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 458 - protein, 139 aa, Artificial Sequence
  Description of Artificial Sequence: Synthetic polypeptide
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 459 - protein, 139 aa, Artificial Sequence
  Description of Artificial Sequence: Synthetic polypeptide
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 460 - protein, 110 aa, Artificial Sequence
  Description of Artificial Sequence: Synthetic polypeptide
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 461 - protein, 108 aa, Artificial Sequence
  Description of Artificial Sequence: Synthetic polypeptide
NAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 462 - protein, 109 aa, Artificial Sequence
  Description of Artificial Sequence: Synthetic polypeptide
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 463 - protein, 129 aa, Artificial Sequence
  Description of Artificial Sequence: Synthetic polypeptide
TALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 464 - protein, 120 aa, Artificial Sequence
  Description of Artificial Sequence: Synthetic polypeptide
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 465 - protein, 125 aa, Artificial Sequence
  Description of Artificial Sequence: Synthetic polypeptide
QADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 466 - protein, 447 aa, Artificial Sequence
  Description of Artificial Sequence: Synthetic polypeptide
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQG

SEQ ID NO: 467 - protein, 1286 aa, Artificial Sequence
  Description of Artificial Sequence: Synthetic polypeptide
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 468 - protein, 839 aa, Artificial Sequence
  Description of Artificial Sequence: Synthetic polypeptide
DDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 469 - protein, 119 aa, Artificial Sequence
  Description of Artificial Sequence: Synthetic polypeptide
DAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 470 - protein, 114 aa, Artificial Sequence
  Description of Artificial Sequence: Synthetic polypeptide
AAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 471 - protein, 120 aa, Artificial Sequence
  Description of Artificial Sequence: Synthetic polypeptide
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 472 - protein, 109 aa, Artificial Sequence
  Description of Artificial Sequence: Synthetic polypeptide
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 473 - protein, 114 aa, Artificial Sequence
  Description of Artificial Sequence: Synthetic polypeptide
ARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 474 - protein, 206 aa, Artificial Sequence
  Description of Artificial Sequence: Synthetic polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 475 - protein, 290 aa, Artificial Sequence
  Description of Artificial Sequence: Synthetic polypeptide
FSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKR

SEQ ID NO: 476 - skipped

SEQ ID NO: 477 - protein, 247 aa, Artificial Sequence
  Description of Artificial Sequence: Synthetic polypeptide
IYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 478 - skipped

SEQ ID NO: 479 - protein, 400 aa, Artificial Sequence
  Description of Artificial Sequence: Synthetic polypeptide
AEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 480 - protein, 163 aa, Artificial Sequence
  Description of Artificial Sequence: Synthetic polypeptide
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 481 - protein, 177 aa, Artificial Sequence
  Description of Artificial Sequence: Synthetic polypeptide
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 482 - protein, 243 aa, Artificial Sequence
  Description of Artificial Sequence: Synthetic polypeptide
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 483 - protein, 343 aa, Artificial Sequence
  Description of Artificial Sequence: Synthetic polypeptide
ANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 484 - protein, 335 aa, Artificial Sequence
  Description of Artificial Sequence: Synthetic polypeptide
SWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 485 - protein, 333 aa, Artificial Sequence
  Description of Artificial Sequence: Synthetic polypeptide
SWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 486 - protein, 334 aa, Artificial Sequence
  Description of Artificial Sequence: Synthetic polypeptide
GYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 487 - protein, 334 aa, Artificial Sequence
  Description of Artificial Sequence: Synthetic polypeptide
SYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 488 - protein, 329 aa, Artificial Sequence
  Description of Artificial Sequence: Synthetic polypeptide
SNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 489 - protein, 319 aa, Artificial Sequence
  Description of Artificial Sequence: Synthetic polypeptide
IIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 490 - protein, 342 aa, Artificial Sequence
  Description of Artificial Sequence: Synthetic polypeptide
QLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 491 - protein, 341 aa, Artificial Sequence
  Description of Artificial Sequence: Synthetic polypeptide
LWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 492 - protein, 318 aa, Artificial Sequence
  Description of Artificial Sequence: Synthetic polypeptide
LGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 493 - protein, 325 aa, Artificial Sequence
  Description of Artificial Sequence: Synthetic polypeptide
ALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 494 - protein, 327 aa, Artificial Sequence
  Description of Artificial Sequence: Synthetic polypeptide
VNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 495 - protein, 329 aa, Artificial Sequence
  Description of Artificial Sequence: Synthetic polypeptide
WSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 496 - protein, 336 aa, Artificial Sequence
  Description of Artificial Sequence: Synthetic polypeptide
YFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 497 - protein, 335 aa, Artificial Sequence
  Description of Artificial Sequence: Synthetic polypeptide
SYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 498 - protein, 382 aa, Artificial Sequence
  Description of Artificial Sequence: Synthetic polypeptide
MPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 499 - protein, 361 aa, Artificial Sequence
  Description of Artificial Sequence: Synthetic polypeptide
NEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 500 - protein, 330 aa, Artificial Sequence
  Description of Artificial Sequence: Synthetic polypeptide
IKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 501 - protein, 336 aa, Artificial Sequence
  Description of Artificial Sequence: Synthetic polypeptide
NWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK